SEQUENCE LISTING (900 sequences)

SEQ ID NO: 1 - protein, 99 aa, Artificial Sequence
  synthetic construct
AEESDNVDSADAEEDDSDVWWGGADTDYADGSEDKVVEVAEEEEVAEVEEEEADDDEDDEDGDEVEEEAEEPYEEATERTTSIATTTTTTTESVEEVVR

SEQ ID NO: 2 - protein, 10 aa, Artificial Sequence
  synthetic construct
AEESDNVDSA

SEQ ID NO: 3 - protein, 10 aa, Artificial Sequence
  synthetic construct
EESDNVDSAD

SEQ ID NO: 4 - protein, 10 aa, Artificial Sequence
  synthetic construct
ESDNVDSADA

SEQ ID NO: 5 - protein, 10 aa, Artificial Sequence
  synthetic construct
SDNVDSADAE

SEQ ID NO: 6 - protein, 10 aa, Artificial Sequence
  synthetic construct
DNVDSADAEE

SEQ ID NO: 7 - protein, 10 aa, Artificial Sequence
  synthetic construct
NVDSADAEED

SEQ ID NO: 8 - protein, 10 aa, Artificial Sequence
  synthetic construct
VDSADAEEDD

SEQ ID NO: 9 - protein, 10 aa, Artificial Sequence
  synthetic construct
DSADAEEDDS

SEQ ID NO: 10 - protein, 10 aa, Artificial Sequence
  synthetic construct
SADAEEDDSD

SEQ ID NO: 11 - protein, 10 aa, Artificial Sequence
  synthetic construct
ADAEEDDSDV

SEQ ID NO: 12 - protein, 10 aa, Artificial Sequence
  synthetic construct
DAEEDDSDVW

SEQ ID NO: 13 - protein, 10 aa, Artificial Sequence
  synthetic construct
AEEDDSDVWW

SEQ ID NO: 14 - protein, 10 aa, Artificial Sequence
  synthetic construct
EEDDSDVWWG

SEQ ID NO: 15 - protein, 10 aa, Artificial Sequence
  synthetic construct
EDDSDVWWGG

SEQ ID NO: 16 - protein, 10 aa, Artificial Sequence
  synthetic construct
DDSDVWWGGA

SEQ ID NO: 17 - protein, 10 aa, Artificial Sequence
  synthetic construct
DSDVWWGGAD

SEQ ID NO: 18 - protein, 10 aa, Artificial Sequence
  synthetic construct
SDVWWGGADT

SEQ ID NO: 19 - protein, 10 aa, Artificial Sequence
  synthetic construct
DVWWGGADTD

SEQ ID NO: 20 - protein, 10 aa, Artificial Sequence
  synthetic construct
VWWGGADTDY

SEQ ID NO: 21 - protein, 10 aa, Artificial Sequence
  synthetic construct
WWGGADTDYA

SEQ ID NO: 22 - protein, 10 aa, Artificial Sequence
  synthetic construct
WGGADTDYAD

SEQ ID NO: 23 - protein, 10 aa, Artificial Sequence
  synthetic construct
GGADTDYADG

SEQ ID NO: 24 - protein, 10 aa, Artificial Sequence
  synthetic construct
GADTDYADGS

SEQ ID NO: 25 - protein, 10 aa, Artificial Sequence
  synthetic construct
ADTDYADGSE

SEQ ID NO: 26 - protein, 10 aa, Artificial Sequence
  synthetic construct
DTDYADGSED

SEQ ID NO: 27 - protein, 10 aa, Artificial Sequence
  synthetic construct
TDYADGSEDK

SEQ ID NO: 28 - protein, 10 aa, Artificial Sequence
  synthetic construct
DYADGSEDKV

SEQ ID NO: 29 - protein, 10 aa, Artificial Sequence
  synthetic construct
YADGSEDKVV

SEQ ID NO: 30 - protein, 10 aa, Artificial Sequence
  synthetic construct
ADGSEDKVVE

SEQ ID NO: 31 - protein, 10 aa, Artificial Sequence
  synthetic construct
DGSEDKVVEV

SEQ ID NO: 32 - protein, 10 aa, Artificial Sequence
  synthetic construct
GSEDKVVEVA

SEQ ID NO: 33 - protein, 10 aa, Artificial Sequence
  synthetic construct
SEDKVVEVAE

SEQ ID NO: 34 - protein, 10 aa, Artificial Sequence
  synthetic construct
EDKVVEVAEE

SEQ ID NO: 35 - protein, 10 aa, Artificial Sequence
  synthetic construct
DKVVEVAEEE

SEQ ID NO: 36 - protein, 10 aa, Artificial Sequence
  synthetic construct
KVVEVAEEEE

SEQ ID NO: 37 - protein, 10 aa, Artificial Sequence
  synthetic construct
VVEVAEEEEV

SEQ ID NO: 38 - protein, 10 aa, Artificial Sequence
  synthetic construct
VEVAEEEEVA

SEQ ID NO: 39 - protein, 10 aa, Artificial Sequence
  synthetic construct
EVAEEEEVAE

SEQ ID NO: 40 - protein, 10 aa, Artificial Sequence
  synthetic construct
VAEEEEVAEV

SEQ ID NO: 41 - protein, 10 aa, Artificial Sequence
  synthetic construct
AEEEEVAEVE

SEQ ID NO: 42 - protein, 10 aa, Artificial Sequence
  synthetic construct
EEEEVAEVEE

SEQ ID NO: 43 - protein, 10 aa, Artificial Sequence
  synthetic construct
EEEVAEVEEE

SEQ ID NO: 44 - protein, 10 aa, Artificial Sequence
  synthetic construct
EEVAEVEEEE

SEQ ID NO: 45 - protein, 10 aa, Artificial Sequence
  synthetic construct
EVAEVEEEEA

SEQ ID NO: 46 - protein, 10 aa, Artificial Sequence
  synthetic construct
VAEVEEEEAD

SEQ ID NO: 47 - protein, 10 aa, Artificial Sequence
  synthetic construct
AEVEEEEADD

SEQ ID NO: 48 - protein, 10 aa, Artificial Sequence
  synthetic construct
EVEEEEADDD

SEQ ID NO: 49 - protein, 10 aa, Artificial Sequence
  synthetic construct
VEEEEADDDE

SEQ ID NO: 50 - protein, 10 aa, Artificial Sequence
  synthetic construct
EEEEADDDED

SEQ ID NO: 51 - protein, 10 aa, Artificial Sequence
  synthetic construct
EEEADDDEDD

SEQ ID NO: 52 - protein, 10 aa, Artificial Sequence
  synthetic construct
EEADDDEDDE

SEQ ID NO: 53 - protein, 10 aa, Artificial Sequence
  synthetic construct
EADDDEDDED

SEQ ID NO: 54 - protein, 10 aa, Artificial Sequence
  synthetic construct
ADDDEDDEDG

SEQ ID NO: 55 - protein, 10 aa, Artificial Sequence
  synthetic construct
DDDEDDEDGD

SEQ ID NO: 56 - protein, 10 aa, Artificial Sequence
  synthetic construct
DDEDDEDGDE

SEQ ID NO: 57 - protein, 10 aa, Artificial Sequence
  synthetic construct
DEDDEDGDEV

SEQ ID NO: 58 - protein, 10 aa, Artificial Sequence
  synthetic construct
EDDEDGDEVE

SEQ ID NO: 59 - protein, 10 aa, Artificial Sequence
  synthetic construct
DDEDGDEVEE

SEQ ID NO: 60 - protein, 10 aa, Artificial Sequence
  synthetic construct
DEDGDEVEEE

SEQ ID NO: 61 - protein, 10 aa, Artificial Sequence
  synthetic construct
EDGDEVEEEA

SEQ ID NO: 62 - protein, 10 aa, Artificial Sequence
  synthetic construct
DGDEVEEEAE

SEQ ID NO: 63 - protein, 10 aa, Artificial Sequence
  synthetic construct
GDEVEEEAEE

SEQ ID NO: 64 - protein, 10 aa, Artificial Sequence
  synthetic construct
DEVEEEAEEP

SEQ ID NO: 65 - protein, 10 aa, Artificial Sequence
  synthetic construct
EVEEEAEEPY

SEQ ID NO: 66 - protein, 10 aa, Artificial Sequence
  synthetic construct
VEEEAEEPYE

SEQ ID NO: 67 - protein, 10 aa, Artificial Sequence
  synthetic construct
EEEAEEPYEE

SEQ ID NO: 68 - protein, 10 aa, Artificial Sequence
  synthetic construct
EEAEEPYEEA

SEQ ID NO: 69 - protein, 10 aa, Artificial Sequence
  synthetic construct
EAEEPYEEAT

SEQ ID NO: 70 - protein, 10 aa, Artificial Sequence
  synthetic construct
AEEPYEEATE

SEQ ID NO: 71 - protein, 10 aa, Artificial Sequence
  synthetic construct
EEPYEEATER

SEQ ID NO: 72 - protein, 10 aa, Artificial Sequence
  synthetic construct
EPYEEATERT

SEQ ID NO: 73 - protein, 10 aa, Artificial Sequence
  synthetic construct
PYEEATERTT

SEQ ID NO: 74 - protein, 10 aa, Artificial Sequence
  synthetic construct
YEEATERTTS

SEQ ID NO: 75 - protein, 10 aa, Artificial Sequence
  synthetic construct
EEATERTTSI

SEQ ID NO: 76 - protein, 10 aa, Artificial Sequence
  synthetic construct
EATERTTSIA

SEQ ID NO: 77 - protein, 10 aa, Artificial Sequence
  synthetic construct
ATERTTSIAT

SEQ ID NO: 78 - protein, 10 aa, Artificial Sequence
  synthetic construct
TERTTSIATT

SEQ ID NO: 79 - protein, 10 aa, Artificial Sequence
  synthetic construct
ERTTSIATTT

SEQ ID NO: 80 - protein, 10 aa, Artificial Sequence
  synthetic construct
RTTSIATTTT

SEQ ID NO: 81 - protein, 10 aa, Artificial Sequence
  synthetic construct
TTSIATTTTT

SEQ ID NO: 82 - protein, 10 aa, Artificial Sequence
  synthetic construct
TSIATTTTTT

SEQ ID NO: 83 - protein, 10 aa, Artificial Sequence
  synthetic construct
SIATTTTTTT

SEQ ID NO: 84 - protein, 10 aa, Artificial Sequence
  synthetic construct
IATTTTTTTE

SEQ ID NO: 85 - protein, 10 aa, Artificial Sequence
  synthetic construct
ATTTTTTTES

SEQ ID NO: 86 - protein, 10 aa, Artificial Sequence
  synthetic construct
TTTTTTTESV

SEQ ID NO: 87 - protein, 10 aa, Artificial Sequence
  synthetic construct
TTTTTTESVE

SEQ ID NO: 88 - protein, 10 aa, Artificial Sequence
  synthetic construct
TTTTTESVEE

SEQ ID NO: 89 - protein, 10 aa, Artificial Sequence
  synthetic construct
TTTTESVEEV

SEQ ID NO: 90 - protein, 10 aa, Artificial Sequence
  synthetic construct
TTTESVEEVV

SEQ ID NO: 91 - protein, 10 aa, Artificial Sequence
  synthetic construct
TTESVEEVVR

SEQ ID NO: 92 - protein, 11 aa, Artificial Sequence
  synthetic construct
AEESDNVDSAD

SEQ ID NO: 93 - protein, 11 aa, Artificial Sequence
  synthetic construct
EESDNVDSADA

SEQ ID NO: 94 - protein, 11 aa, Artificial Sequence
  synthetic construct
ESDNVDSADAE

SEQ ID NO: 95 - protein, 11 aa, Artificial Sequence
  synthetic construct
SDNVDSADAEE

SEQ ID NO: 96 - protein, 11 aa, Artificial Sequence
  synthetic construct
DNVDSADAEED

SEQ ID NO: 97 - protein, 11 aa, Artificial Sequence
  synthetic construct
NVDSADAEEDD

SEQ ID NO: 98 - protein, 11 aa, Artificial Sequence
  synthetic construct
VDSADAEEDDS

SEQ ID NO: 99 - protein, 11 aa, Artificial Sequence
  synthetic construct
DSADAEEDDSD

SEQ ID NO: 100 - protein, 11 aa, Artificial Sequence
  synthetic construct
SADAEEDDSDV

SEQ ID NO: 101 - protein, 11 aa, Artificial Sequence
  synthetic construct
ADAEEDDSDVW

SEQ ID NO: 102 - protein, 11 aa, Artificial Sequence
  synthetic construct
DAEEDDSDVWW

SEQ ID NO: 103 - protein, 11 aa, Artificial Sequence
  synthetic construct
AEEDDSDVWWG

SEQ ID NO: 104 - protein, 11 aa, Artificial Sequence
  synthetic construct
EEDDSDVWWGG

SEQ ID NO: 105 - protein, 11 aa, Artificial Sequence
  synthetic construct
EDDSDVWWGGA

SEQ ID NO: 106 - protein, 11 aa, Artificial Sequence
  synthetic construct
DDSDVWWGGAD

SEQ ID NO: 107 - protein, 11 aa, Artificial Sequence
  synthetic construct
DSDVWWGGADT

SEQ ID NO: 108 - protein, 11 aa, Artificial Sequence
  synthetic construct
SDVWWGGADTD

SEQ ID NO: 109 - protein, 11 aa, Artificial Sequence
  synthetic construct
DVWWGGADTDY

SEQ ID NO: 110 - protein, 11 aa, Artificial Sequence
  synthetic construct
VWWGGADTDYA

SEQ ID NO: 111 - protein, 11 aa, Artificial Sequence
  synthetic construct
WWGGADTDYAD

SEQ ID NO: 112 - protein, 11 aa, Artificial Sequence
  synthetic construct
WGGADTDYADG

SEQ ID NO: 113 - protein, 11 aa, Artificial Sequence
  synthetic construct
GGADTDYADGS

SEQ ID NO: 114 - protein, 11 aa, Artificial Sequence
  synthetic construct
GADTDYADGSE

SEQ ID NO: 115 - protein, 11 aa, Artificial Sequence
  synthetic construct
ADTDYADGSED

SEQ ID NO: 116 - protein, 11 aa, Artificial Sequence
  synthetic construct
DTDYADGSEDK

SEQ ID NO: 117 - protein, 11 aa, Artificial Sequence
  synthetic construct
TDYADGSEDKV

SEQ ID NO: 118 - protein, 11 aa, Artificial Sequence
  synthetic construct
DYADGSEDKVV

SEQ ID NO: 119 - protein, 11 aa, Artificial Sequence
  synthetic construct
YADGSEDKVVE

SEQ ID NO: 120 - protein, 11 aa, Artificial Sequence
  synthetic construct
ADGSEDKVVEV

SEQ ID NO: 121 - protein, 11 aa, Artificial Sequence
  synthetic construct
DGSEDKVVEVA

SEQ ID NO: 122 - protein, 11 aa, Artificial Sequence
  synthetic construct
GSEDKVVEVAE

SEQ ID NO: 123 - protein, 11 aa, Artificial Sequence
  synthetic construct
SEDKVVEVAEE

SEQ ID NO: 124 - protein, 11 aa, Artificial Sequence
  synthetic construct
EDKVVEVAEEE

SEQ ID NO: 125 - protein, 11 aa, Artificial Sequence
  synthetic construct
DKVVEVAEEEE

SEQ ID NO: 126 - protein, 11 aa, Artificial Sequence
  synthetic construct
KVVEVAEEEEV

SEQ ID NO: 127 - protein, 11 aa, Artificial Sequence
  synthetic construct
VVEVAEEEEVA

SEQ ID NO: 128 - protein, 11 aa, Artificial Sequence
  synthetic construct
VEVAEEEEVAE

SEQ ID NO: 129 - protein, 11 aa, Artificial Sequence
  synthetic construct
EVAEEEEVAEV

SEQ ID NO: 130 - protein, 11 aa, Artificial Sequence
  synthetic construct
VAEEEEVAEVE

SEQ ID NO: 131 - protein, 11 aa, Artificial Sequence
  synthetic construct
AEEEEVAEVEE

SEQ ID NO: 132 - protein, 11 aa, Artificial Sequence
  synthetic construct
EEEEVAEVEEE

SEQ ID NO: 133 - protein, 11 aa, Artificial Sequence
  synthetic construct
EEEVAEVEEEE

SEQ ID NO: 134 - protein, 11 aa, Artificial Sequence
  synthetic construct
EEVAEVEEEEA

SEQ ID NO: 135 - protein, 11 aa, Artificial Sequence
  synthetic construct
EVAEVEEEEAD

SEQ ID NO: 136 - protein, 11 aa, Artificial Sequence
  synthetic construct
VAEVEEEEADD

SEQ ID NO: 137 - protein, 11 aa, Artificial Sequence
  synthetic construct
AEVEEEEADDD

SEQ ID NO: 138 - protein, 11 aa, Artificial Sequence
  synthetic construct
EVEEEEADDDE

SEQ ID NO: 139 - protein, 11 aa, Artificial Sequence
  synthetic construct
VEEEEADDDED

SEQ ID NO: 140 - protein, 11 aa, Artificial Sequence
  synthetic construct
EEEEADDDEDD

SEQ ID NO: 141 - protein, 11 aa, Artificial Sequence
  synthetic construct
EEEADDDEDDE

SEQ ID NO: 142 - protein, 11 aa, Artificial Sequence
  synthetic construct
EEADDDEDDED

SEQ ID NO: 143 - protein, 11 aa, Artificial Sequence
  synthetic construct
EADDDEDDEDG

SEQ ID NO: 144 - protein, 11 aa, Artificial Sequence
  synthetic construct
ADDDEDDEDGD

SEQ ID NO: 145 - protein, 11 aa, Artificial Sequence
  synthetic construct
DDDEDDEDGDE

SEQ ID NO: 146 - protein, 11 aa, Artificial Sequence
  synthetic construct
DDEDDEDGDEV

SEQ ID NO: 147 - protein, 11 aa, Artificial Sequence
  synthetic construct
DEDDEDGDEVE

SEQ ID NO: 148 - protein, 11 aa, Artificial Sequence
  synthetic construct
EDDEDGDEVEE

SEQ ID NO: 149 - protein, 11 aa, Artificial Sequence
  synthetic construct
DDEDGDEVEEE

SEQ ID NO: 150 - protein, 11 aa, Artificial Sequence
  synthetic construct
DEDGDEVEEEA

SEQ ID NO: 151 - protein, 11 aa, Artificial Sequence
  synthetic construct
EDGDEVEEEAE

SEQ ID NO: 152 - protein, 11 aa, Artificial Sequence
  synthetic construct
DGDEVEEEAEE

SEQ ID NO: 153 - protein, 11 aa, Artificial Sequence
  synthetic construct
GDEVEEEAEEP

SEQ ID NO: 154 - protein, 11 aa, Artificial Sequence
  synthetic construct
DEVEEEAEEPY

SEQ ID NO: 155 - protein, 11 aa, Artificial Sequence
  synthetic construct
EVEEEAEEPYE

SEQ ID NO: 156 - protein, 11 aa, Artificial Sequence
  synthetic construct
VEEEAEEPYEE

SEQ ID NO: 157 - protein, 11 aa, Artificial Sequence
  synthetic construct
EEEAEEPYEEA

SEQ ID NO: 158 - protein, 11 aa, Artificial Sequence
  synthetic construct
EEAEEPYEEAT

SEQ ID NO: 159 - protein, 11 aa, Artificial Sequence
  synthetic construct
EAEEPYEEATE

SEQ ID NO: 160 - protein, 11 aa, Artificial Sequence
  synthetic construct
AEEPYEEATER

SEQ ID NO: 161 - protein, 11 aa, Artificial Sequence
  synthetic construct
EEPYEEATERT

SEQ ID NO: 162 - protein, 11 aa, Artificial Sequence
  synthetic construct
EPYEEATERTT

SEQ ID NO: 163 - protein, 11 aa, Artificial Sequence
  synthetic construct
PYEEATERTTS

SEQ ID NO: 164 - protein, 11 aa, Artificial Sequence
  synthetic construct
YEEATERTTSI

SEQ ID NO: 165 - protein, 11 aa, Artificial Sequence
  synthetic construct
EEATERTTSIA

SEQ ID NO: 166 - protein, 11 aa, Artificial Sequence
  synthetic construct
EATERTTSIAT

SEQ ID NO: 167 - protein, 11 aa, Artificial Sequence
  synthetic construct
ATERTTSIATT

SEQ ID NO: 168 - protein, 11 aa, Artificial Sequence
  synthetic construct
TERTTSIATTT

SEQ ID NO: 169 - protein, 11 aa, Artificial Sequence
  synthetic construct
ERTTSIATTTT

SEQ ID NO: 170 - protein, 11 aa, Artificial Sequence
  synthetic construct
RTTSIATTTTT

SEQ ID NO: 171 - protein, 11 aa, Artificial Sequence
  synthetic construct
TTSIATTTTTT

SEQ ID NO: 172 - protein, 11 aa, Artificial Sequence
  synthetic construct
TSIATTTTTTT

SEQ ID NO: 173 - protein, 11 aa, Artificial Sequence
  synthetic construct
SIATTTTTTTE

SEQ ID NO: 174 - protein, 11 aa, Artificial Sequence
  synthetic construct
IATTTTTTTES

SEQ ID NO: 175 - protein, 11 aa, Artificial Sequence
  synthetic construct
ATTTTTTTESV

SEQ ID NO: 176 - protein, 11 aa, Artificial Sequence
  synthetic construct
TTTTTTTESVE

SEQ ID NO: 177 - protein, 11 aa, Artificial Sequence
  synthetic construct
TTTTTTESVEE

SEQ ID NO: 178 - protein, 11 aa, Artificial Sequence
  synthetic construct
TTTTTESVEEV

SEQ ID NO: 179 - protein, 11 aa, Artificial Sequence
  synthetic construct
TTTTESVEEVV

SEQ ID NO: 180 - protein, 11 aa, Artificial Sequence
  synthetic construct
TTTESVEEVVR

SEQ ID NO: 181 - protein, 12 aa, Artificial Sequence
  synthetic construct
AEESDNVDSADA

SEQ ID NO: 182 - protein, 12 aa, Artificial Sequence
  synthetic construct
EESDNVDSADAE

SEQ ID NO: 183 - protein, 12 aa, Artificial Sequence
  synthetic construct
ESDNVDSADAEE

SEQ ID NO: 184 - protein, 12 aa, Artificial Sequence
  synthetic construct
SDNVDSADAEED

SEQ ID NO: 185 - protein, 12 aa, Artificial Sequence
  synthetic construct
DNVDSADAEEDD

SEQ ID NO: 186 - protein, 12 aa, Artificial Sequence
  synthetic construct
NVDSADAEEDDS

SEQ ID NO: 187 - protein, 12 aa, Artificial Sequence
  synthetic construct
VDSADAEEDDSD

SEQ ID NO: 188 - protein, 12 aa, Artificial Sequence
  synthetic construct
DSADAEEDDSDV

SEQ ID NO: 189 - protein, 12 aa, Artificial Sequence
  synthetic construct
SADAEEDDSDVW

SEQ ID NO: 190 - protein, 12 aa, Artificial Sequence
  synthetic construct
ADAEEDDSDVWW

SEQ ID NO: 191 - protein, 12 aa, Artificial Sequence
  synthetic construct
DAEEDDSDVWWG

SEQ ID NO: 192 - protein, 12 aa, Artificial Sequence
  synthetic construct
AEEDDSDVWWGG

SEQ ID NO: 193 - protein, 12 aa, Artificial Sequence
  synthetic construct
EEDDSDVWWGGA

SEQ ID NO: 194 - protein, 12 aa, Artificial Sequence
  synthetic construct
EDDSDVWWGGAD

SEQ ID NO: 195 - protein, 12 aa, Artificial Sequence
  synthetic construct
DDSDVWWGGADT

SEQ ID NO: 196 - protein, 12 aa, Artificial Sequence
  synthetic construct
DSDVWWGGADTD

SEQ ID NO: 197 - protein, 12 aa, Artificial Sequence
  synthetic construct
SDVWWGGADTDY

SEQ ID NO: 198 - protein, 12 aa, Artificial Sequence
  synthetic construct
DVWWGGADTDYA

SEQ ID NO: 199 - protein, 12 aa, Artificial Sequence
  synthetic construct
VWWGGADTDYAD

SEQ ID NO: 200 - protein, 12 aa, Artificial Sequence
  synthetic construct
WWGGADTDYADG

SEQ ID NO: 201 - protein, 12 aa, Artificial Sequence
  synthetic construct
WGGADTDYADGS

SEQ ID NO: 202 - protein, 12 aa, Artificial Sequence
  synthetic construct
GGADTDYADGSE

SEQ ID NO: 203 - protein, 12 aa, Artificial Sequence
  synthetic construct
GADTDYADGSED

SEQ ID NO: 204 - protein, 12 aa, Artificial Sequence
  synthetic construct
ADTDYADGSEDK

SEQ ID NO: 205 - protein, 12 aa, Artificial Sequence
  synthetic construct
DTDYADGSEDKV

SEQ ID NO: 206 - protein, 12 aa, Artificial Sequence
  synthetic construct
TDYADGSEDKVV

SEQ ID NO: 207 - protein, 12 aa, Artificial Sequence
  synthetic construct
DYADGSEDKVVE

SEQ ID NO: 208 - protein, 12 aa, Artificial Sequence
  synthetic construct
YADGSEDKVVEV

SEQ ID NO: 209 - protein, 12 aa, Artificial Sequence
  synthetic construct
ADGSEDKVVEVA

SEQ ID NO: 210 - protein, 12 aa, Artificial Sequence
  synthetic construct
DGSEDKVVEVAE

SEQ ID NO: 211 - protein, 12 aa, Artificial Sequence
  synthetic construct
GSEDKVVEVAEE

SEQ ID NO: 212 - protein, 12 aa, Artificial Sequence
  synthetic construct
SEDKVVEVAEEE

SEQ ID NO: 213 - protein, 12 aa, Artificial Sequence
  synthetic construct
EDKVVEVAEEEE

SEQ ID NO: 214 - protein, 12 aa, Artificial Sequence
  synthetic construct
DKVVEVAEEEEV

SEQ ID NO: 215 - protein, 12 aa, Artificial Sequence
  synthetic construct
KVVEVAEEEEVA

SEQ ID NO: 216 - protein, 12 aa, Artificial Sequence
  synthetic construct
VVEVAEEEEVAE

SEQ ID NO: 217 - protein, 12 aa, Artificial Sequence
  synthetic construct
VEVAEEEEVAEV

SEQ ID NO: 218 - protein, 12 aa, Artificial Sequence
  synthetic construct
EVAEEEEVAEVE

SEQ ID NO: 219 - protein, 12 aa, Artificial Sequence
  synthetic construct
VAEEEEVAEVEE

SEQ ID NO: 220 - protein, 12 aa, Artificial Sequence
  synthetic construct
AEEEEVAEVEEE

SEQ ID NO: 221 - protein, 12 aa, Artificial Sequence
  synthetic construct
EEEEVAEVEEEE

SEQ ID NO: 222 - protein, 12 aa, Artificial Sequence
  synthetic construct
EEEVAEVEEEEA

SEQ ID NO: 223 - protein, 12 aa, Artificial Sequence
  synthetic construct
EEVAEVEEEEAD

SEQ ID NO: 224 - protein, 12 aa, Artificial Sequence
  synthetic construct
EVAEVEEEEADD

SEQ ID NO: 225 - protein, 12 aa, Artificial Sequence
  synthetic construct
VAEVEEEEADDD

SEQ ID NO: 226 - protein, 12 aa, Artificial Sequence
  synthetic construct
AEVEEEEADDDE

SEQ ID NO: 227 - protein, 12 aa, Artificial Sequence
  synthetic construct
EVEEEEADDDED

SEQ ID NO: 228 - protein, 12 aa, Artificial Sequence
  synthetic construct
VEEEEADDDEDD

SEQ ID NO: 229 - protein, 12 aa, Artificial Sequence
  synthetic construct
EEEEADDDEDDE

SEQ ID NO: 230 - protein, 12 aa, Artificial Sequence
  synthetic construct
EEEADDDEDDED

SEQ ID NO: 231 - protein, 12 aa, Artificial Sequence
  synthetic construct
EEADDDEDDEDG

SEQ ID NO: 232 - protein, 12 aa, Artificial Sequence
  synthetic construct
EADDDEDDEDGD

SEQ ID NO: 233 - protein, 12 aa, Artificial Sequence
  synthetic construct
ADDDEDDEDGDE

SEQ ID NO: 234 - protein, 12 aa, Artificial Sequence
  synthetic construct
DDDEDDEDGDEV

SEQ ID NO: 235 - protein, 12 aa, Artificial Sequence
  synthetic construct
DDEDDEDGDEVE

SEQ ID NO: 236 - protein, 12 aa, Artificial Sequence
  synthetic construct
DEDDEDGDEVEE

SEQ ID NO: 237 - protein, 12 aa, Artificial Sequence
  synthetic construct
EDDEDGDEVEEE

SEQ ID NO: 238 - protein, 12 aa, Artificial Sequence
  synthetic construct
DDEDGDEVEEEA

SEQ ID NO: 239 - protein, 12 aa, Artificial Sequence
  synthetic construct
DEDGDEVEEEAE

SEQ ID NO: 240 - protein, 12 aa, Artificial Sequence
  synthetic construct
EDGDEVEEEAEE

SEQ ID NO: 241 - protein, 12 aa, Artificial Sequence
  synthetic construct
DGDEVEEEAEEP

SEQ ID NO: 242 - protein, 12 aa, Artificial Sequence
  synthetic construct
GDEVEEEAEEPY

SEQ ID NO: 243 - protein, 12 aa, Artificial Sequence
  synthetic construct
DEVEEEAEEPYE

SEQ ID NO: 244 - protein, 12 aa, Artificial Sequence
  synthetic construct
EVEEEAEEPYEE

SEQ ID NO: 245 - protein, 12 aa, Artificial Sequence
  synthetic construct
VEEEAEEPYEEA

SEQ ID NO: 246 - protein, 12 aa, Artificial Sequence
  synthetic construct
EEEAEEPYEEAT

SEQ ID NO: 247 - protein, 12 aa, Artificial Sequence
  synthetic construct
EEAEEPYEEATE

SEQ ID NO: 248 - protein, 12 aa, Artificial Sequence
  synthetic construct
EAEEPYEEATER

SEQ ID NO: 249 - protein, 12 aa, Artificial Sequence
  synthetic construct
AEEPYEEATERT

SEQ ID NO: 250 - protein, 12 aa, Artificial Sequence
  synthetic construct
EEPYEEATERTT

SEQ ID NO: 251 - protein, 12 aa, Artificial Sequence
  synthetic construct
EPYEEATERTTS

SEQ ID NO: 252 - protein, 12 aa, Artificial Sequence
  synthetic construct
PYEEATERTTSI

SEQ ID NO: 253 - protein, 12 aa, Artificial Sequence
  synthetic construct
YEEATERTTSIA

SEQ ID NO: 254 - protein, 12 aa, Artificial Sequence
  synthetic construct
EEATERTTSIAT

SEQ ID NO: 255 - protein, 12 aa, Artificial Sequence
  synthetic construct
EATERTTSIATT

SEQ ID NO: 256 - protein, 12 aa, Artificial Sequence
  synthetic construct
ATERTTSIATTT

SEQ ID NO: 257 - protein, 12 aa, Artificial Sequence
  synthetic construct
TERTTSIATTTT

SEQ ID NO: 258 - protein, 12 aa, Artificial Sequence
  synthetic construct
ERTTSIATTTTT

SEQ ID NO: 259 - protein, 12 aa, Artificial Sequence
  synthetic construct
RTTSIATTTTTT

SEQ ID NO: 260 - protein, 12 aa, Artificial Sequence
  synthetic construct
TTSIATTTTTTT

SEQ ID NO: 261 - protein, 12 aa, Artificial Sequence
  synthetic construct
TSIATTTTTTTE

SEQ ID NO: 262 - protein, 12 aa, Artificial Sequence
  synthetic construct
SIATTTTTTTES

SEQ ID NO: 263 - protein, 12 aa, Artificial Sequence
  synthetic construct
IATTTTTTTESV

SEQ ID NO: 264 - protein, 12 aa, Artificial Sequence
  synthetic construct
ATTTTTTTESVE

SEQ ID NO: 265 - protein, 12 aa, Artificial Sequence
  synthetic construct
TTTTTTTESVEE

SEQ ID NO: 266 - protein, 12 aa, Artificial Sequence
  synthetic construct
TTTTTTESVEEV

SEQ ID NO: 267 - protein, 12 aa, Artificial Sequence
  synthetic construct
TTTTTESVEEVV

SEQ ID NO: 268 - protein, 12 aa, Artificial Sequence
  synthetic construct
TTTTESVEEVVR

SEQ ID NO: 269 - protein, 13 aa, Artificial Sequence
  synthetic construct
AEESDNVDSADAE

SEQ ID NO: 270 - protein, 13 aa, Artificial Sequence
  synthetic construct
EESDNVDSADAEE

SEQ ID NO: 271 - protein, 13 aa, Artificial Sequence
  synthetic construct
ESDNVDSADAEED

SEQ ID NO: 272 - protein, 13 aa, Artificial Sequence
  synthetic construct
SDNVDSADAEEDD

SEQ ID NO: 273 - protein, 13 aa, Artificial Sequence
  synthetic construct
DNVDSADAEEDDS

SEQ ID NO: 274 - protein, 13 aa, Artificial Sequence
  synthetic construct
NVDSADAEEDDSD

SEQ ID NO: 275 - protein, 13 aa, Artificial Sequence
  synthetic construct
VDSADAEEDDSDV

SEQ ID NO: 276 - protein, 13 aa, Artificial Sequence
  synthetic construct
DSADAEEDDSDVW

SEQ ID NO: 277 - protein, 13 aa, Artificial Sequence
  synthetic construct
SADAEEDDSDVWW

SEQ ID NO: 278 - protein, 13 aa, Artificial Sequence
  synthetic construct
ADAEEDDSDVWWG

SEQ ID NO: 279 - protein, 13 aa, Artificial Sequence
  synthetic construct
DAEEDDSDVWWGG

SEQ ID NO: 280 - protein, 13 aa, Artificial Sequence
  synthetic construct
AEEDDSDVWWGGA

SEQ ID NO: 281 - protein, 13 aa, Artificial Sequence
  synthetic construct
EEDDSDVWWGGAD

SEQ ID NO: 282 - protein, 13 aa, Artificial Sequence
  synthetic construct
EDDSDVWWGGADT

SEQ ID NO: 283 - protein, 13 aa, Artificial Sequence
  synthetic construct
DDSDVWWGGADTD

SEQ ID NO: 284 - protein, 13 aa, Artificial Sequence
  synthetic construct
DSDVWWGGADTDY

SEQ ID NO: 285 - protein, 13 aa, Artificial Sequence
  synthetic construct
SDVWWGGADTDYA

SEQ ID NO: 286 - protein, 13 aa, Artificial Sequence
  synthetic construct
DVWWGGADTDYAD

SEQ ID NO: 287 - protein, 13 aa, Artificial Sequence
  synthetic construct
VWWGGADTDYADG

SEQ ID NO: 288 - protein, 13 aa, Artificial Sequence
  synthetic construct
WWGGADTDYADGS

SEQ ID NO: 289 - protein, 13 aa, Artificial Sequence
  synthetic construct
WGGADTDYADGSE

SEQ ID NO: 290 - protein, 13 aa, Artificial Sequence
  synthetic construct
GGADTDYADGSED

SEQ ID NO: 291 - protein, 13 aa, Artificial Sequence
  synthetic construct
GADTDYADGSEDK

SEQ ID NO: 292 - protein, 13 aa, Artificial Sequence
  synthetic construct
ADTDYADGSEDKV

SEQ ID NO: 293 - protein, 13 aa, Artificial Sequence
  synthetic construct
DTDYADGSEDKVV

SEQ ID NO: 294 - protein, 13 aa, Artificial Sequence
  synthetic construct
TDYADGSEDKVVE

SEQ ID NO: 295 - protein, 13 aa, Artificial Sequence
  synthetic construct
DYADGSEDKVVEV

SEQ ID NO: 296 - protein, 13 aa, Artificial Sequence
  synthetic construct
YADGSEDKVVEVA

SEQ ID NO: 297 - protein, 13 aa, Artificial Sequence
  synthetic construct
ADGSEDKVVEVAE

SEQ ID NO: 298 - protein, 13 aa, Artificial Sequence
  synthetic construct
DGSEDKVVEVAEE

SEQ ID NO: 299 - protein, 13 aa, Artificial Sequence
  synthetic construct
GSEDKVVEVAEEE

SEQ ID NO: 300 - protein, 13 aa, Artificial Sequence
  synthetic construct
SEDKVVEVAEEEE

SEQ ID NO: 301 - protein, 13 aa, Artificial Sequence
  synthetic construct
EDKVVEVAEEEEV

SEQ ID NO: 302 - protein, 13 aa, Artificial Sequence
  synthetic construct
DKVVEVAEEEEVA

SEQ ID NO: 303 - protein, 13 aa, Artificial Sequence
  synthetic construct
KVVEVAEEEEVAE

SEQ ID NO: 304 - protein, 13 aa, Artificial Sequence
  synthetic construct
VVEVAEEEEVAEV

SEQ ID NO: 305 - protein, 13 aa, Artificial Sequence
  synthetic construct
VEVAEEEEVAEVE

SEQ ID NO: 306 - protein, 13 aa, Artificial Sequence
  synthetic construct
EVAEEEEVAEVEE

SEQ ID NO: 307 - protein, 13 aa, Artificial Sequence
  synthetic construct
VAEEEEVAEVEEE

SEQ ID NO: 308 - protein, 13 aa, Artificial Sequence
  synthetic construct
AEEEEVAEVEEEE

SEQ ID NO: 309 - protein, 13 aa, Artificial Sequence
  synthetic construct
EEEEVAEVEEEEA

SEQ ID NO: 310 - protein, 13 aa, Artificial Sequence
  synthetic construct
EEEVAEVEEEEAD

SEQ ID NO: 311 - protein, 13 aa, Artificial Sequence
  synthetic construct
EEVAEVEEEEADD

SEQ ID NO: 312 - protein, 13 aa, Artificial Sequence
  synthetic construct
EVAEVEEEEADDD

SEQ ID NO: 313 - protein, 13 aa, Artificial Sequence
  synthetic construct
VAEVEEEEADDDE

SEQ ID NO: 314 - protein, 13 aa, Artificial Sequence
  synthetic construct
AEVEEEEADDDED

SEQ ID NO: 315 - protein, 13 aa, Artificial Sequence
  synthetic construct
EVEEEEADDDEDD

SEQ ID NO: 316 - protein, 13 aa, Artificial Sequence
  synthetic construct
VEEEEADDDEDDE

SEQ ID NO: 317 - protein, 13 aa, Artificial Sequence
  synthetic construct
EEEEADDDEDDED

SEQ ID NO: 318 - protein, 13 aa, Artificial Sequence
  synthetic construct
EEEADDDEDDEDG

SEQ ID NO: 319 - protein, 13 aa, Artificial Sequence
  synthetic construct
EEADDDEDDEDGD

SEQ ID NO: 320 - protein, 13 aa, Artificial Sequence
  synthetic construct
EADDDEDDEDGDE

SEQ ID NO: 321 - protein, 13 aa, Artificial Sequence
  synthetic construct
ADDDEDDEDGDEV

SEQ ID NO: 322 - protein, 13 aa, Artificial Sequence
  synthetic construct
DDDEDDEDGDEVE

SEQ ID NO: 323 - protein, 13 aa, Artificial Sequence
  synthetic construct
DDEDDEDGDEVEE

SEQ ID NO: 324 - protein, 13 aa, Artificial Sequence
  synthetic construct
DEDDEDGDEVEEE

SEQ ID NO: 325 - protein, 13 aa, Artificial Sequence
  synthetic construct
EDDEDGDEVEEEA

SEQ ID NO: 326 - protein, 13 aa, Artificial Sequence
  synthetic construct
DDEDGDEVEEEAE

SEQ ID NO: 327 - protein, 13 aa, Artificial Sequence
  synthetic construct
DEDGDEVEEEAEE

SEQ ID NO: 328 - protein, 13 aa, Artificial Sequence
  synthetic construct
EDGDEVEEEAEEP

SEQ ID NO: 329 - protein, 13 aa, Artificial Sequence
  synthetic construct
DGDEVEEEAEEPY

SEQ ID NO: 330 - protein, 13 aa, Artificial Sequence
  synthetic construct
GDEVEEEAEEPYE

SEQ ID NO: 331 - protein, 13 aa, Artificial Sequence
  synthetic construct
DEVEEEAEEPYEE

SEQ ID NO: 332 - protein, 13 aa, Artificial Sequence
  synthetic construct
EVEEEAEEPYEEA

SEQ ID NO: 333 - protein, 13 aa, Artificial Sequence
  synthetic construct
VEEEAEEPYEEAT

SEQ ID NO: 334 - protein, 13 aa, Artificial Sequence
  synthetic construct
EEEAEEPYEEATE

SEQ ID NO: 335 - protein, 13 aa, Artificial Sequence
  synthetic construct
EEAEEPYEEATER

SEQ ID NO: 336 - protein, 13 aa, Artificial Sequence
  synthetic construct
EAEEPYEEATERT

SEQ ID NO: 337 - protein, 13 aa, Artificial Sequence
  synthetic construct
AEEPYEEATERTT

SEQ ID NO: 338 - protein, 13 aa, Artificial Sequence
  synthetic construct
EEPYEEATERTTS

SEQ ID NO: 339 - protein, 13 aa, Artificial Sequence
  synthetic construct
EPYEEATERTTSI

SEQ ID NO: 340 - protein, 13 aa, Artificial Sequence
  synthetic construct
PYEEATERTTSIA

SEQ ID NO: 341 - protein, 13 aa, Artificial Sequence
  synthetic construct
YEEATERTTSIAT

SEQ ID NO: 342 - protein, 13 aa, Artificial Sequence
  synthetic construct
EEATERTTSIATT

SEQ ID NO: 343 - protein, 13 aa, Artificial Sequence
  synthetic construct
EATERTTSIATTT

SEQ ID NO: 344 - protein, 13 aa, Artificial Sequence
  synthetic construct
ATERTTSIATTTT

SEQ ID NO: 345 - protein, 13 aa, Artificial Sequence
  synthetic construct
TERTTSIATTTTT

SEQ ID NO: 346 - protein, 13 aa, Artificial Sequence
  synthetic construct
ERTTSIATTTTTT

SEQ ID NO: 347 - protein, 13 aa, Artificial Sequence
  synthetic construct
RTTSIATTTTTTT

SEQ ID NO: 348 - protein, 13 aa, Artificial Sequence
  synthetic construct
TTSIATTTTTTTE

SEQ ID NO: 349 - protein, 13 aa, Artificial Sequence
  synthetic construct
TSIATTTTTTTES

SEQ ID NO: 350 - protein, 13 aa, Artificial Sequence
  synthetic construct
SIATTTTTTTESV

SEQ ID NO: 351 - protein, 13 aa, Artificial Sequence
  synthetic construct
IATTTTTTTESVE

SEQ ID NO: 352 - protein, 13 aa, Artificial Sequence
  synthetic construct
ATTTTTTTESVEE

SEQ ID NO: 353 - protein, 13 aa, Artificial Sequence
  synthetic construct
TTTTTTTESVEEV

SEQ ID NO: 354 - protein, 13 aa, Artificial Sequence
  synthetic construct
TTTTTTESVEEVV

SEQ ID NO: 355 - protein, 13 aa, Artificial Sequence
  synthetic construct
TTTTTESVEEVVR

SEQ ID NO: 356 - protein, 14 aa, Artificial Sequence
  synthetic construct
AEESDNVDSADAEE

SEQ ID NO: 357 - protein, 14 aa, Artificial Sequence
  synthetic construct
EESDNVDSADAEED

SEQ ID NO: 358 - protein, 14 aa, Artificial Sequence
  synthetic construct
ESDNVDSADAEEDD

SEQ ID NO: 359 - protein, 14 aa, Artificial Sequence
  synthetic construct
SDNVDSADAEEDDS

SEQ ID NO: 360 - protein, 14 aa, Artificial Sequence
  synthetic construct
DNVDSADAEEDDSD

SEQ ID NO: 361 - protein, 14 aa, Artificial Sequence
  synthetic construct
NVDSADAEEDDSDV

SEQ ID NO: 362 - protein, 14 aa, Artificial Sequence
  synthetic construct
VDSADAEEDDSDVW

SEQ ID NO: 363 - protein, 14 aa, Artificial Sequence
  synthetic construct
DSADAEEDDSDVWW

SEQ ID NO: 364 - protein, 14 aa, Artificial Sequence
  synthetic construct
SADAEEDDSDVWWG

SEQ ID NO: 365 - protein, 14 aa, Artificial Sequence
  synthetic construct
ADAEEDDSDVWWGG

SEQ ID NO: 366 - protein, 14 aa, Artificial Sequence
  synthetic construct
DAEEDDSDVWWGGA

SEQ ID NO: 367 - protein, 14 aa, Artificial Sequence
  synthetic construct
AEEDDSDVWWGGAD

SEQ ID NO: 368 - protein, 14 aa, Artificial Sequence
  synthetic construct
EEDDSDVWWGGADT

SEQ ID NO: 369 - protein, 14 aa, Artificial Sequence
  synthetic construct
EDDSDVWWGGADTD

SEQ ID NO: 370 - protein, 14 aa, Artificial Sequence
  synthetic construct
DDSDVWWGGADTDY

SEQ ID NO: 371 - protein, 14 aa, Artificial Sequence
  synthetic construct
DSDVWWGGADTDYA

SEQ ID NO: 372 - protein, 14 aa, Artificial Sequence
  synthetic construct
SDVWWGGADTDYAD

SEQ ID NO: 373 - protein, 14 aa, Artificial Sequence
  synthetic construct
DVWWGGADTDYADG

SEQ ID NO: 374 - protein, 14 aa, Artificial Sequence
  synthetic construct
VWWGGADTDYADGS

SEQ ID NO: 375 - protein, 14 aa, Artificial Sequence
  synthetic construct
WWGGADTDYADGSE

SEQ ID NO: 376 - protein, 14 aa, Artificial Sequence
  synthetic construct
WGGADTDYADGSED

SEQ ID NO: 377 - protein, 14 aa, Artificial Sequence
  synthetic construct
GGADTDYADGSEDK

SEQ ID NO: 378 - protein, 14 aa, Artificial Sequence
  synthetic construct
GADTDYADGSEDKV

SEQ ID NO: 379 - protein, 14 aa, Artificial Sequence
  synthetic construct
ADTDYADGSEDKVV

SEQ ID NO: 380 - protein, 14 aa, Artificial Sequence
  synthetic construct
DTDYADGSEDKVVE

SEQ ID NO: 381 - protein, 14 aa, Artificial Sequence
  synthetic construct
TDYADGSEDKVVEV

SEQ ID NO: 382 - protein, 14 aa, Artificial Sequence
  synthetic construct
DYADGSEDKVVEVA

SEQ ID NO: 383 - protein, 14 aa, Artificial Sequence
  synthetic construct
YADGSEDKVVEVAE

SEQ ID NO: 384 - protein, 14 aa, Artificial Sequence
  synthetic construct
ADGSEDKVVEVAEE

SEQ ID NO: 385 - protein, 14 aa, Artificial Sequence
  synthetic construct
DGSEDKVVEVAEEE

SEQ ID NO: 386 - protein, 14 aa, Artificial Sequence
  synthetic construct
GSEDKVVEVAEEEE

SEQ ID NO: 387 - protein, 14 aa, Artificial Sequence
  synthetic construct
SEDKVVEVAEEEEV

SEQ ID NO: 388 - protein, 14 aa, Artificial Sequence
  synthetic construct
EDKVVEVAEEEEVA

SEQ ID NO: 389 - protein, 14 aa, Artificial Sequence
  synthetic construct
DKVVEVAEEEEVAE

SEQ ID NO: 390 - protein, 14 aa, Artificial Sequence
  synthetic construct
KVVEVAEEEEVAEV

SEQ ID NO: 391 - protein, 14 aa, Artificial Sequence
  synthetic construct
VVEVAEEEEVAEVE

SEQ ID NO: 392 - protein, 14 aa, Artificial Sequence
  synthetic construct
VEVAEEEEVAEVEE

SEQ ID NO: 393 - protein, 14 aa, Artificial Sequence
  synthetic construct
EVAEEEEVAEVEEE

SEQ ID NO: 394 - protein, 14 aa, Artificial Sequence
  synthetic construct
VAEEEEVAEVEEEE

SEQ ID NO: 395 - protein, 14 aa, Artificial Sequence
  synthetic construct
AEEEEVAEVEEEEA

SEQ ID NO: 396 - protein, 14 aa, Artificial Sequence
  synthetic construct
EEEEVAEVEEEEAD

SEQ ID NO: 397 - protein, 14 aa, Artificial Sequence
  synthetic construct
EEEVAEVEEEEADD

SEQ ID NO: 398 - protein, 14 aa, Artificial Sequence
  synthetic construct
EEVAEVEEEEADDD

SEQ ID NO: 399 - protein, 14 aa, Artificial Sequence
  synthetic construct
EVAEVEEEEADDDE

SEQ ID NO: 400 - protein, 14 aa, Artificial Sequence
  synthetic construct
VAEVEEEEADDDED

SEQ ID NO: 401 - protein, 14 aa, Artificial Sequence
  synthetic construct
AEVEEEEADDDEDD

SEQ ID NO: 402 - protein, 14 aa, Artificial Sequence
  synthetic construct
EVEEEEADDDEDDE

SEQ ID NO: 403 - protein, 14 aa, Artificial Sequence
  synthetic construct
VEEEEADDDEDDED

SEQ ID NO: 404 - protein, 14 aa, Artificial Sequence
  synthetic construct
EEEEADDDEDDEDG

SEQ ID NO: 405 - protein, 14 aa, Artificial Sequence
  synthetic construct
EEEADDDEDDEDGD

SEQ ID NO: 406 - protein, 14 aa, Artificial Sequence
  synthetic construct
EEADDDEDDEDGDE

SEQ ID NO: 407 - protein, 14 aa, Artificial Sequence
  synthetic construct
EADDDEDDEDGDEV

SEQ ID NO: 408 - protein, 14 aa, Artificial Sequence
  synthetic construct
ADDDEDDEDGDEVE

SEQ ID NO: 409 - protein, 14 aa, Artificial Sequence
  synthetic construct
DDDEDDEDGDEVEE

SEQ ID NO: 410 - protein, 14 aa, Artificial Sequence
  synthetic construct
DDEDDEDGDEVEEE

SEQ ID NO: 411 - protein, 14 aa, Artificial Sequence
  synthetic construct
DEDDEDGDEVEEEA

SEQ ID NO: 412 - protein, 14 aa, Artificial Sequence
  synthetic construct
EDDEDGDEVEEEAE

SEQ ID NO: 413 - protein, 14 aa, Artificial Sequence
  synthetic construct
DDEDGDEVEEEAEE

SEQ ID NO: 414 - protein, 14 aa, Artificial Sequence
  synthetic construct
DEDGDEVEEEAEEP

SEQ ID NO: 415 - protein, 14 aa, Artificial Sequence
  synthetic construct
EDGDEVEEEAEEPY

SEQ ID NO: 416 - protein, 14 aa, Artificial Sequence
  synthetic construct
DGDEVEEEAEEPYE

SEQ ID NO: 417 - protein, 14 aa, Artificial Sequence
  synthetic construct
GDEVEEEAEEPYEE

SEQ ID NO: 418 - protein, 14 aa, Artificial Sequence
  synthetic construct
DEVEEEAEEPYEEA

SEQ ID NO: 419 - protein, 14 aa, Artificial Sequence
  synthetic construct
EVEEEAEEPYEEAT

SEQ ID NO: 420 - protein, 14 aa, Artificial Sequence
  synthetic construct
VEEEAEEPYEEATE

SEQ ID NO: 421 - protein, 14 aa, Artificial Sequence
  synthetic construct
EEEAEEPYEEATER

SEQ ID NO: 422 - protein, 14 aa, Artificial Sequence
  synthetic construct
EEAEEPYEEATERT

SEQ ID NO: 423 - protein, 14 aa, Artificial Sequence
  synthetic construct
EAEEPYEEATERTT

SEQ ID NO: 424 - protein, 14 aa, Artificial Sequence
  synthetic construct
AEEPYEEATERTTS

SEQ ID NO: 425 - protein, 14 aa, Artificial Sequence
  synthetic construct
EEPYEEATERTTSI

SEQ ID NO: 426 - protein, 14 aa, Artificial Sequence
  synthetic construct
EPYEEATERTTSIA

SEQ ID NO: 427 - protein, 14 aa, Artificial Sequence
  synthetic construct
PYEEATERTTSIAT

SEQ ID NO: 428 - protein, 14 aa, Artificial Sequence
  synthetic construct
YEEATERTTSIATT

SEQ ID NO: 429 - protein, 14 aa, Artificial Sequence
  synthetic construct
EEATERTTSIATTT

SEQ ID NO: 430 - protein, 14 aa, Artificial Sequence
  synthetic construct
EATERTTSIATTTT

SEQ ID NO: 431 - protein, 14 aa, Artificial Sequence
  synthetic construct
ATERTTSIATTTTT

SEQ ID NO: 432 - protein, 14 aa, Artificial Sequence
  synthetic construct
TERTTSIATTTTTT

SEQ ID NO: 433 - protein, 14 aa, Artificial Sequence
  synthetic construct
ERTTSIATTTTTTT

SEQ ID NO: 434 - protein, 14 aa, Artificial Sequence
  synthetic construct
RTTSIATTTTTTTE

SEQ ID NO: 435 - protein, 14 aa, Artificial Sequence
  synthetic construct
TTSIATTTTTTTES

SEQ ID NO: 436 - protein, 14 aa, Artificial Sequence
  synthetic construct
TSIATTTTTTTESV

SEQ ID NO: 437 - protein, 14 aa, Artificial Sequence
  synthetic construct
SIATTTTTTTESVE

SEQ ID NO: 438 - protein, 14 aa, Artificial Sequence
  synthetic construct
IATTTTTTTESVEE

SEQ ID NO: 439 - protein, 14 aa, Artificial Sequence
  synthetic construct
ATTTTTTTESVEEV

SEQ ID NO: 440 - protein, 14 aa, Artificial Sequence
  synthetic construct
TTTTTTTESVEEVV

SEQ ID NO: 441 - protein, 14 aa, Artificial Sequence
  synthetic construct
TTTTTTESVEEVVR

SEQ ID NO: 442 - protein, 98 aa, Artificial Sequence
  synthetic construct
EEPPRFIKEPKDQIGVSGGVASFVCQATGDPKPRVTWNKKGKKVNSQRFETIEFDESAGAVLRIQPLRTPRDENVYECVAQNSVGEITVHAKLTVLRE

SEQ ID NO: 443 - protein, 10 aa, Artificial Sequence
  synthetic construct
EEPPRFIKEP

SEQ ID NO: 444 - protein, 10 aa, Artificial Sequence
  synthetic construct
EPPRFIKEPK

SEQ ID NO: 445 - protein, 10 aa, Artificial Sequence
  synthetic construct
PPRFIKEPKD

SEQ ID NO: 446 - protein, 10 aa, Artificial Sequence
  synthetic construct
PRFIKEPKDQ

SEQ ID NO: 447 - protein, 10 aa, Artificial Sequence
  synthetic construct
RFIKEPKDQI

SEQ ID NO: 448 - protein, 10 aa, Artificial Sequence
  synthetic construct
FIKEPKDQIG

SEQ ID NO: 449 - protein, 10 aa, Artificial Sequence
  synthetic construct
IKEPKDQIGV

SEQ ID NO: 450 - protein, 10 aa, Artificial Sequence
  synthetic construct
KEPKDQIGVS

SEQ ID NO: 451 - protein, 10 aa, Artificial Sequence
  synthetic construct
EPKDQIGVSG

SEQ ID NO: 452 - protein, 10 aa, Artificial Sequence
  synthetic construct
PKDQIGVSGG

SEQ ID NO: 453 - protein, 10 aa, Artificial Sequence
  synthetic construct
KDQIGVSGGV

SEQ ID NO: 454 - protein, 10 aa, Artificial Sequence
  synthetic construct
DQIGVSGGVA

SEQ ID NO: 455 - protein, 10 aa, Artificial Sequence
  synthetic construct
QIGVSGGVAS

SEQ ID NO: 456 - protein, 10 aa, Artificial Sequence
  synthetic construct
IGVSGGVASF

SEQ ID NO: 457 - protein, 10 aa, Artificial Sequence
  synthetic construct
GVSGGVASFV

SEQ ID NO: 458 - protein, 10 aa, Artificial Sequence
  synthetic construct
VSGGVASFVC

SEQ ID NO: 459 - protein, 10 aa, Artificial Sequence
  synthetic construct
SGGVASFVCQ

SEQ ID NO: 460 - protein, 10 aa, Artificial Sequence
  synthetic construct
GGVASFVCQA

SEQ ID NO: 461 - protein, 10 aa, Artificial Sequence
  synthetic construct
GVASFVCQAT

SEQ ID NO: 462 - protein, 10 aa, Artificial Sequence
  synthetic construct
VASFVCQATG

SEQ ID NO: 463 - protein, 10 aa, Artificial Sequence
  synthetic construct
ASFVCQATGD

SEQ ID NO: 464 - protein, 10 aa, Artificial Sequence
  synthetic construct
SFVCQATGDP

SEQ ID NO: 465 - protein, 10 aa, Artificial Sequence
  synthetic construct
FVCQATGDPK

SEQ ID NO: 466 - protein, 10 aa, Artificial Sequence
  synthetic construct
VCQATGDPKP

SEQ ID NO: 467 - protein, 10 aa, Artificial Sequence
  synthetic construct
CQATGDPKPR

SEQ ID NO: 468 - protein, 10 aa, Artificial Sequence
  synthetic construct
QATGDPKPRV

SEQ ID NO: 469 - protein, 10 aa, Artificial Sequence
  synthetic construct
ATGDPKPRVT

SEQ ID NO: 470 - protein, 10 aa, Artificial Sequence
  synthetic construct
TGDPKPRVTW

SEQ ID NO: 471 - protein, 10 aa, Artificial Sequence
  synthetic construct
GDPKPRVTWN

SEQ ID NO: 472 - protein, 10 aa, Artificial Sequence
  synthetic construct
DPKPRVTWNK

SEQ ID NO: 473 - protein, 10 aa, Artificial Sequence
  synthetic construct
PKPRVTWNKK

SEQ ID NO: 474 - protein, 10 aa, Artificial Sequence
  synthetic construct
KPRVTWNKKG

SEQ ID NO: 475 - protein, 10 aa, Artificial Sequence
  synthetic construct
PRVTWNKKGK

SEQ ID NO: 476 - protein, 10 aa, Artificial Sequence
  synthetic construct
RVTWNKKGKK

SEQ ID NO: 477 - protein, 10 aa, Artificial Sequence
  synthetic construct
VTWNKKGKKV

SEQ ID NO: 478 - protein, 10 aa, Artificial Sequence
  synthetic construct
TWNKKGKKVN

SEQ ID NO: 479 - protein, 10 aa, Artificial Sequence
  synthetic construct
WNKKGKKVNS

SEQ ID NO: 480 - protein, 10 aa, Artificial Sequence
  synthetic construct
NKKGKKVNSQ

SEQ ID NO: 481 - protein, 10 aa, Artificial Sequence
  synthetic construct
KKGKKVNSQR

SEQ ID NO: 482 - protein, 10 aa, Artificial Sequence
  synthetic construct
KGKKVNSQRF

SEQ ID NO: 483 - protein, 10 aa, Artificial Sequence
  synthetic construct
GKKVNSQRFE

SEQ ID NO: 484 - protein, 10 aa, Artificial Sequence
  synthetic construct
KKVNSQRFET

SEQ ID NO: 485 - protein, 10 aa, Artificial Sequence
  synthetic construct
KVNSQRFETI

SEQ ID NO: 486 - protein, 10 aa, Artificial Sequence
  synthetic construct
VNSQRFETIE

SEQ ID NO: 487 - protein, 10 aa, Artificial Sequence
  synthetic construct
NSQRFETIEF

SEQ ID NO: 488 - protein, 10 aa, Artificial Sequence
  synthetic construct
SQRFETIEFD

SEQ ID NO: 489 - protein, 10 aa, Artificial Sequence
  synthetic construct
QRFETIEFDE

SEQ ID NO: 490 - protein, 10 aa, Artificial Sequence
  synthetic construct
RFETIEFDES

SEQ ID NO: 491 - protein, 10 aa, Artificial Sequence
  synthetic construct
FETIEFDESA

SEQ ID NO: 492 - protein, 10 aa, Artificial Sequence
  synthetic construct
ETIEFDESAG

SEQ ID NO: 493 - protein, 10 aa, Artificial Sequence
  synthetic construct
TIEFDESAGA

SEQ ID NO: 494 - protein, 10 aa, Artificial Sequence
  synthetic construct
IEFDESAGAV

SEQ ID NO: 495 - protein, 10 aa, Artificial Sequence
  synthetic construct
EFDESAGAVL

SEQ ID NO: 496 - protein, 10 aa, Artificial Sequence
  synthetic construct
FDESAGAVLR

SEQ ID NO: 497 - protein, 10 aa, Artificial Sequence
  synthetic construct
DESAGAVLRI

SEQ ID NO: 498 - protein, 10 aa, Artificial Sequence
  synthetic construct
ESAGAVLRIQ

SEQ ID NO: 499 - protein, 10 aa, Artificial Sequence
  synthetic construct
SAGAVLRIQP

SEQ ID NO: 500 - protein, 10 aa, Artificial Sequence
  synthetic construct
AGAVLRIQPL

SEQ ID NO: 501 - protein, 10 aa, Artificial Sequence
  synthetic construct
GAVLRIQPLR

SEQ ID NO: 502 - protein, 10 aa, Artificial Sequence
  synthetic construct
AVLRIQPLRT

SEQ ID NO: 503 - protein, 10 aa, Artificial Sequence
  synthetic construct
VLRIQPLRTP

SEQ ID NO: 504 - protein, 10 aa, Artificial Sequence
  synthetic construct
LRIQPLRTPR

SEQ ID NO: 505 - protein, 10 aa, Artificial Sequence
  synthetic construct
RIQPLRTPRD

SEQ ID NO: 506 - protein, 10 aa, Artificial Sequence
  synthetic construct
IQPLRTPRDE

SEQ ID NO: 507 - protein, 10 aa, Artificial Sequence
  synthetic construct
QPLRTPRDEN

SEQ ID NO: 508 - protein, 10 aa, Artificial Sequence
  synthetic construct
PLRTPRDENV

SEQ ID NO: 509 - protein, 10 aa, Artificial Sequence
  synthetic construct
LRTPRDENVY

SEQ ID NO: 510 - protein, 10 aa, Artificial Sequence
  synthetic construct
RTPRDENVYE

SEQ ID NO: 511 - protein, 10 aa, Artificial Sequence
  synthetic construct
TPRDENVYEC

SEQ ID NO: 512 - protein, 10 aa, Artificial Sequence
  synthetic construct
PRDENVYECV

SEQ ID NO: 513 - protein, 10 aa, Artificial Sequence
  synthetic construct
RDENVYECVA

SEQ ID NO: 514 - protein, 10 aa, Artificial Sequence
  synthetic construct
DENVYECVAQ

SEQ ID NO: 515 - protein, 10 aa, Artificial Sequence
  synthetic construct
ENVYECVAQN

SEQ ID NO: 516 - protein, 10 aa, Artificial Sequence
  synthetic construct
NVYECVAQNS

SEQ ID NO: 517 - protein, 10 aa, Artificial Sequence
  synthetic construct
VYECVAQNSV

SEQ ID NO: 518 - protein, 10 aa, Artificial Sequence
  synthetic construct
YECVAQNSVG

SEQ ID NO: 519 - protein, 10 aa, Artificial Sequence
  synthetic construct
ECVAQNSVGE

SEQ ID NO: 520 - protein, 10 aa, Artificial Sequence
  synthetic construct
CVAQNSVGEI

SEQ ID NO: 521 - protein, 10 aa, Artificial Sequence
  synthetic construct
VAQNSVGEIT

SEQ ID NO: 522 - protein, 10 aa, Artificial Sequence
  synthetic construct
AQNSVGEITV

SEQ ID NO: 523 - protein, 10 aa, Artificial Sequence
  synthetic construct
QNSVGEITVH

SEQ ID NO: 524 - protein, 10 aa, Artificial Sequence
  synthetic construct
NSVGEITVHA

SEQ ID NO: 525 - protein, 10 aa, Artificial Sequence
  synthetic construct
SVGEITVHAK

SEQ ID NO: 526 - protein, 10 aa, Artificial Sequence
  synthetic construct
VGEITVHAKL

SEQ ID NO: 527 - protein, 10 aa, Artificial Sequence
  synthetic construct
GEITVHAKLT

SEQ ID NO: 528 - protein, 10 aa, Artificial Sequence
  synthetic construct
EITVHAKLTV

SEQ ID NO: 529 - protein, 10 aa, Artificial Sequence
  synthetic construct
ITVHAKLTVL

SEQ ID NO: 530 - protein, 10 aa, Artificial Sequence
  synthetic construct
TVHAKLTVLR

SEQ ID NO: 531 - protein, 10 aa, Artificial Sequence
  synthetic construct
VHAKLTVLRE

SEQ ID NO: 532 - protein, 11 aa, Artificial Sequence
  synthetic construct
EEPPRFIKEPK

SEQ ID NO: 533 - protein, 11 aa, Artificial Sequence
  synthetic construct
EPPRFIKEPKD

SEQ ID NO: 534 - protein, 11 aa, Artificial Sequence
  synthetic construct
PPRFIKEPKDQ

SEQ ID NO: 535 - protein, 11 aa, Artificial Sequence
  synthetic construct
PRFIKEPKDQI

SEQ ID NO: 536 - protein, 11 aa, Artificial Sequence
  synthetic construct
RFIKEPKDQIG

SEQ ID NO: 537 - protein, 11 aa, Artificial Sequence
  synthetic construct
FIKEPKDQIGV

SEQ ID NO: 538 - protein, 11 aa, Artificial Sequence
  synthetic construct
IKEPKDQIGVS

SEQ ID NO: 539 - protein, 11 aa, Artificial Sequence
  synthetic construct
KEPKDQIGVSG

SEQ ID NO: 540 - protein, 11 aa, Artificial Sequence
  synthetic construct
EPKDQIGVSGG

SEQ ID NO: 541 - protein, 11 aa, Artificial Sequence
  synthetic construct
PKDQIGVSGGV

SEQ ID NO: 542 - protein, 11 aa, Artificial Sequence
  synthetic construct
KDQIGVSGGVA

SEQ ID NO: 543 - protein, 11 aa, Artificial Sequence
  synthetic construct
DQIGVSGGVAS

SEQ ID NO: 544 - protein, 11 aa, Artificial Sequence
  synthetic construct
QIGVSGGVASF

SEQ ID NO: 545 - protein, 11 aa, Artificial Sequence
  synthetic construct
IGVSGGVASFV

SEQ ID NO: 546 - protein, 11 aa, Artificial Sequence
  synthetic construct
GVSGGVASFVC

SEQ ID NO: 547 - protein, 11 aa, Artificial Sequence
  synthetic construct
VSGGVASFVCQ

SEQ ID NO: 548 - protein, 11 aa, Artificial Sequence
  synthetic construct
SGGVASFVCQA

SEQ ID NO: 549 - protein, 11 aa, Artificial Sequence
  synthetic construct
GGVASFVCQAT

SEQ ID NO: 550 - protein, 11 aa, Artificial Sequence
  synthetic construct
GVASFVCQATG

SEQ ID NO: 551 - protein, 11 aa, Artificial Sequence
  synthetic construct
VASFVCQATGD

SEQ ID NO: 552 - protein, 11 aa, Artificial Sequence
  synthetic construct
ASFVCQATGDP

SEQ ID NO: 553 - protein, 11 aa, Artificial Sequence
  synthetic construct
SFVCQATGDPK

SEQ ID NO: 554 - protein, 11 aa, Artificial Sequence
  synthetic construct
FVCQATGDPKP

SEQ ID NO: 555 - protein, 11 aa, Artificial Sequence
  synthetic construct
VCQATGDPKPR

SEQ ID NO: 556 - protein, 11 aa, Artificial Sequence
  synthetic construct
CQATGDPKPRV

SEQ ID NO: 557 - protein, 11 aa, Artificial Sequence
  synthetic construct
QATGDPKPRVT

SEQ ID NO: 558 - protein, 11 aa, Artificial Sequence
  synthetic construct
ATGDPKPRVTW

SEQ ID NO: 559 - protein, 11 aa, Artificial Sequence
  synthetic construct
TGDPKPRVTWN

SEQ ID NO: 560 - protein, 11 aa, Artificial Sequence
  synthetic construct
GDPKPRVTWNK

SEQ ID NO: 561 - protein, 11 aa, Artificial Sequence
  synthetic construct
DPKPRVTWNKK

SEQ ID NO: 562 - protein, 11 aa, Artificial Sequence
  synthetic construct
PKPRVTWNKKG

SEQ ID NO: 563 - protein, 11 aa, Artificial Sequence
  synthetic construct
KPRVTWNKKGK

SEQ ID NO: 564 - protein, 11 aa, Artificial Sequence
  synthetic construct
PRVTWNKKGKK

SEQ ID NO: 565 - protein, 11 aa, Artificial Sequence
  synthetic construct
RVTWNKKGKKV

SEQ ID NO: 566 - protein, 11 aa, Artificial Sequence
  synthetic construct
VTWNKKGKKVN

SEQ ID NO: 567 - protein, 11 aa, Artificial Sequence
  synthetic construct
TWNKKGKKVNS

SEQ ID NO: 568 - protein, 11 aa, Artificial Sequence
  synthetic construct
WNKKGKKVNSQ

SEQ ID NO: 569 - protein, 11 aa, Artificial Sequence
  synthetic construct
NKKGKKVNSQR

SEQ ID NO: 570 - protein, 11 aa, Artificial Sequence
  synthetic construct
KKGKKVNSQRF

SEQ ID NO: 571 - protein, 11 aa, Artificial Sequence
  synthetic construct
KGKKVNSQRFE

SEQ ID NO: 572 - protein, 11 aa, Artificial Sequence
  synthetic construct
GKKVNSQRFET

SEQ ID NO: 573 - protein, 11 aa, Artificial Sequence
  synthetic construct
KKVNSQRFETI

SEQ ID NO: 574 - protein, 11 aa, Artificial Sequence
  synthetic construct
KVNSQRFETIE

SEQ ID NO: 575 - protein, 11 aa, Artificial Sequence
  synthetic construct
VNSQRFETIEF

SEQ ID NO: 576 - protein, 11 aa, Artificial Sequence
  synthetic construct
NSQRFETIEFD

SEQ ID NO: 577 - protein, 11 aa, Artificial Sequence
  synthetic construct
SQRFETIEFDE

SEQ ID NO: 578 - protein, 11 aa, Artificial Sequence
  synthetic construct
QRFETIEFDES

SEQ ID NO: 579 - protein, 11 aa, Artificial Sequence
  synthetic construct
RFETIEFDESA

SEQ ID NO: 580 - protein, 11 aa, Artificial Sequence
  synthetic construct
FETIEFDESAG

SEQ ID NO: 581 - protein, 11 aa, Artificial Sequence
  synthetic construct
ETIEFDESAGA

SEQ ID NO: 582 - protein, 11 aa, Artificial Sequence
  synthetic construct
TIEFDESAGAV

SEQ ID NO: 583 - protein, 11 aa, Artificial Sequence
  synthetic construct
IEFDESAGAVL

SEQ ID NO: 584 - protein, 11 aa, Artificial Sequence
  synthetic construct
EFDESAGAVLR

SEQ ID NO: 585 - protein, 11 aa, Artificial Sequence
  synthetic construct
FDESAGAVLRI

SEQ ID NO: 586 - protein, 11 aa, Artificial Sequence
  synthetic construct
DESAGAVLRIQ

SEQ ID NO: 587 - protein, 11 aa, Artificial Sequence
  synthetic construct
ESAGAVLRIQP

SEQ ID NO: 588 - protein, 11 aa, Artificial Sequence
  synthetic construct
SAGAVLRIQPL

SEQ ID NO: 589 - protein, 11 aa, Artificial Sequence
  synthetic construct
AGAVLRIQPLR

SEQ ID NO: 590 - protein, 11 aa, Artificial Sequence
  synthetic construct
GAVLRIQPLRT

SEQ ID NO: 591 - protein, 11 aa, Artificial Sequence
  synthetic construct
AVLRIQPLRTP

SEQ ID NO: 592 - protein, 11 aa, Artificial Sequence
  synthetic construct
VLRIQPLRTPR

SEQ ID NO: 593 - protein, 11 aa, Artificial Sequence
  synthetic construct
LRIQPLRTPRD

SEQ ID NO: 594 - protein, 11 aa, Artificial Sequence
  synthetic construct
RIQPLRTPRDE

SEQ ID NO: 595 - protein, 11 aa, Artificial Sequence
  synthetic construct
IQPLRTPRDEN

SEQ ID NO: 596 - protein, 11 aa, Artificial Sequence
  synthetic construct
QPLRTPRDENV

SEQ ID NO: 597 - protein, 11 aa, Artificial Sequence
  synthetic construct
PLRTPRDENVY

SEQ ID NO: 598 - protein, 11 aa, Artificial Sequence
  synthetic construct
LRTPRDENVYE

SEQ ID NO: 599 - protein, 11 aa, Artificial Sequence
  synthetic construct
RTPRDENVYEC

SEQ ID NO: 600 - protein, 11 aa, Artificial Sequence
  synthetic construct
TPRDENVYECV

SEQ ID NO: 601 - protein, 11 aa, Artificial Sequence
  synthetic construct
PRDENVYECVA

SEQ ID NO: 602 - protein, 11 aa, Artificial Sequence
  synthetic construct
RDENVYECVAQ

SEQ ID NO: 603 - protein, 11 aa, Artificial Sequence
  synthetic construct
DENVYECVAQN

SEQ ID NO: 604 - protein, 11 aa, Artificial Sequence
  synthetic construct
ENVYECVAQNS

SEQ ID NO: 605 - protein, 11 aa, Artificial Sequence
  synthetic construct
NVYECVAQNSV

SEQ ID NO: 606 - protein, 11 aa, Artificial Sequence
  synthetic construct
VYECVAQNSVG

SEQ ID NO: 607 - protein, 11 aa, Artificial Sequence
  synthetic construct
YECVAQNSVGE

SEQ ID NO: 608 - protein, 11 aa, Artificial Sequence
  synthetic construct
ECVAQNSVGEI

SEQ ID NO: 609 - protein, 11 aa, Artificial Sequence
  synthetic construct
CVAQNSVGEIT

SEQ ID NO: 610 - protein, 11 aa, Artificial Sequence
  synthetic construct
VAQNSVGEITV

SEQ ID NO: 611 - protein, 11 aa, Artificial Sequence
  synthetic construct
AQNSVGEITVH

SEQ ID NO: 612 - protein, 11 aa, Artificial Sequence
  synthetic construct
QNSVGEITVHA

SEQ ID NO: 613 - protein, 11 aa, Artificial Sequence
  synthetic construct
NSVGEITVHAK

SEQ ID NO: 614 - protein, 11 aa, Artificial Sequence
  synthetic construct
SVGEITVHAKL

SEQ ID NO: 615 - protein, 11 aa, Artificial Sequence
  synthetic construct
VGEITVHAKLT

SEQ ID NO: 616 - protein, 11 aa, Artificial Sequence
  synthetic construct
GEITVHAKLTV

SEQ ID NO: 617 - protein, 11 aa, Artificial Sequence
  synthetic construct
EITVHAKLTVL

SEQ ID NO: 618 - protein, 11 aa, Artificial Sequence
  synthetic construct
ITVHAKLTVLR

SEQ ID NO: 619 - protein, 11 aa, Artificial Sequence
  synthetic construct
TVHAKLTVLRE

SEQ ID NO: 620 - protein, 12 aa, Artificial Sequence
  synthetic construct
EEPPRFIKEPKD

SEQ ID NO: 621 - protein, 12 aa, Artificial Sequence
  synthetic construct
EPPRFIKEPKDQ

SEQ ID NO: 622 - protein, 12 aa, Artificial Sequence
  synthetic construct
PPRFIKEPKDQI

SEQ ID NO: 623 - protein, 12 aa, Artificial Sequence
  synthetic construct
PRFIKEPKDQIG

SEQ ID NO: 624 - protein, 12 aa, Artificial Sequence
  synthetic construct
RFIKEPKDQIGV

SEQ ID NO: 625 - protein, 12 aa, Artificial Sequence
  synthetic construct
FIKEPKDQIGVS

SEQ ID NO: 626 - protein, 12 aa, Artificial Sequence
  synthetic construct
IKEPKDQIGVSG

SEQ ID NO: 627 - protein, 12 aa, Artificial Sequence
  synthetic construct
KEPKDQIGVSGG

SEQ ID NO: 628 - protein, 12 aa, Artificial Sequence
  synthetic construct
EPKDQIGVSGGV

SEQ ID NO: 629 - protein, 12 aa, Artificial Sequence
  synthetic construct
PKDQIGVSGGVA

SEQ ID NO: 630 - protein, 12 aa, Artificial Sequence
  synthetic construct
KDQIGVSGGVAS

SEQ ID NO: 631 - protein, 12 aa, Artificial Sequence
  synthetic construct
DQIGVSGGVASF

SEQ ID NO: 632 - protein, 12 aa, Artificial Sequence
  synthetic construct
QIGVSGGVASFV

SEQ ID NO: 633 - protein, 12 aa, Artificial Sequence
  synthetic construct
IGVSGGVASFVC

SEQ ID NO: 634 - protein, 12 aa, Artificial Sequence
  synthetic construct
GVSGGVASFVCQ

SEQ ID NO: 635 - protein, 12 aa, Artificial Sequence
  synthetic construct
VSGGVASFVCQA

SEQ ID NO: 636 - protein, 12 aa, Artificial Sequence
  synthetic construct
SGGVASFVCQAT

SEQ ID NO: 637 - protein, 12 aa, Artificial Sequence
  synthetic construct
GGVASFVCQATG

SEQ ID NO: 638 - protein, 12 aa, Artificial Sequence
  synthetic construct
GVASFVCQATGD

SEQ ID NO: 639 - protein, 12 aa, Artificial Sequence
  synthetic construct
VASFVCQATGDP

SEQ ID NO: 640 - protein, 12 aa, Artificial Sequence
  synthetic construct
ASFVCQATGDPK

SEQ ID NO: 641 - protein, 12 aa, Artificial Sequence
  synthetic construct
SFVCQATGDPKP

SEQ ID NO: 642 - protein, 12 aa, Artificial Sequence
  synthetic construct
FVCQATGDPKPR

SEQ ID NO: 643 - protein, 12 aa, Artificial Sequence
  synthetic construct
VCQATGDPKPRV

SEQ ID NO: 644 - protein, 12 aa, Artificial Sequence
  synthetic construct
CQATGDPKPRVT

SEQ ID NO: 645 - protein, 12 aa, Artificial Sequence
  synthetic construct
QATGDPKPRVTW

SEQ ID NO: 646 - protein, 12 aa, Artificial Sequence
  synthetic construct
ATGDPKPRVTWN

SEQ ID NO: 647 - protein, 12 aa, Artificial Sequence
  synthetic construct
TGDPKPRVTWNK

SEQ ID NO: 648 - protein, 12 aa, Artificial Sequence
  synthetic construct
GDPKPRVTWNKK

SEQ ID NO: 649 - protein, 12 aa, Artificial Sequence
  synthetic construct
DPKPRVTWNKKG

SEQ ID NO: 650 - protein, 12 aa, Artificial Sequence
  synthetic construct
PKPRVTWNKKGK

SEQ ID NO: 651 - protein, 12 aa, Artificial Sequence
  synthetic construct
KPRVTWNKKGKK

SEQ ID NO: 652 - protein, 12 aa, Artificial Sequence
  synthetic construct
PRVTWNKKGKKV

SEQ ID NO: 653 - protein, 12 aa, Artificial Sequence
  synthetic construct
RVTWNKKGKKVN

SEQ ID NO: 654 - protein, 12 aa, Artificial Sequence
  synthetic construct
VTWNKKGKKVNS

SEQ ID NO: 655 - protein, 12 aa, Artificial Sequence
  synthetic construct
TWNKKGKKVNSQ

SEQ ID NO: 656 - protein, 12 aa, Artificial Sequence
  synthetic construct
WNKKGKKVNSQR

SEQ ID NO: 657 - protein, 12 aa, Artificial Sequence
  synthetic construct
NKKGKKVNSQRF

SEQ ID NO: 658 - protein, 12 aa, Artificial Sequence
  synthetic construct
KKGKKVNSQRFE

SEQ ID NO: 659 - protein, 12 aa, Artificial Sequence
  synthetic construct
KGKKVNSQRFET

SEQ ID NO: 660 - protein, 12 aa, Artificial Sequence
  synthetic construct
GKKVNSQRFETI

SEQ ID NO: 661 - protein, 12 aa, Artificial Sequence
  synthetic construct
KKVNSQRFETIE

SEQ ID NO: 662 - protein, 12 aa, Artificial Sequence
  synthetic construct
KVNSQRFETIEF

SEQ ID NO: 663 - protein, 12 aa, Artificial Sequence
  synthetic construct
VNSQRFETIEFD

SEQ ID NO: 664 - protein, 12 aa, Artificial Sequence
  synthetic construct
NSQRFETIEFDE

SEQ ID NO: 665 - protein, 12 aa, Artificial Sequence
  synthetic construct
SQRFETIEFDES

SEQ ID NO: 666 - protein, 12 aa, Artificial Sequence
  synthetic construct
QRFETIEFDESA

SEQ ID NO: 667 - protein, 12 aa, Artificial Sequence
  synthetic construct
RFETIEFDESAG

SEQ ID NO: 668 - protein, 12 aa, Artificial Sequence
  synthetic construct
FETIEFDESAGA

SEQ ID NO: 669 - protein, 12 aa, Artificial Sequence
  synthetic construct
ETIEFDESAGAV

SEQ ID NO: 670 - protein, 12 aa, Artificial Sequence
  synthetic construct
TIEFDESAGAVL

SEQ ID NO: 671 - protein, 12 aa, Artificial Sequence
  synthetic construct
IEFDESAGAVLR

SEQ ID NO: 672 - protein, 12 aa, Artificial Sequence
  synthetic construct
EFDESAGAVLRI

SEQ ID NO: 673 - protein, 12 aa, Artificial Sequence
  synthetic construct
FDESAGAVLRIQ

SEQ ID NO: 674 - protein, 12 aa, Artificial Sequence
  synthetic construct
DESAGAVLRIQP

SEQ ID NO: 675 - protein, 12 aa, Artificial Sequence
  synthetic construct
ESAGAVLRIQPL

SEQ ID NO: 676 - protein, 12 aa, Artificial Sequence
  synthetic construct
SAGAVLRIQPLR

SEQ ID NO: 677 - protein, 12 aa, Artificial Sequence
  synthetic construct
AGAVLRIQPLRT

SEQ ID NO: 678 - protein, 12 aa, Artificial Sequence
  synthetic construct
GAVLRIQPLRTP

SEQ ID NO: 679 - protein, 12 aa, Artificial Sequence
  synthetic construct
AVLRIQPLRTPR

SEQ ID NO: 680 - protein, 12 aa, Artificial Sequence
  synthetic construct
VLRIQPLRTPRD

SEQ ID NO: 681 - protein, 12 aa, Artificial Sequence
  synthetic construct
LRIQPLRTPRDE

SEQ ID NO: 682 - protein, 12 aa, Artificial Sequence
  synthetic construct
RIQPLRTPRDEN

SEQ ID NO: 683 - protein, 12 aa, Artificial Sequence
  synthetic construct
IQPLRTPRDENV

SEQ ID NO: 684 - protein, 12 aa, Artificial Sequence
  synthetic construct
QPLRTPRDENVY

SEQ ID NO: 685 - protein, 12 aa, Artificial Sequence
  synthetic construct
PLRTPRDENVYE

SEQ ID NO: 686 - protein, 12 aa, Artificial Sequence
  synthetic construct
LRTPRDENVYEC

SEQ ID NO: 687 - protein, 12 aa, Artificial Sequence
  synthetic construct
RTPRDENVYECV

SEQ ID NO: 688 - protein, 12 aa, Artificial Sequence
  synthetic construct
TPRDENVYECVA

SEQ ID NO: 689 - protein, 12 aa, Artificial Sequence
  synthetic construct
PRDENVYECVAQ

SEQ ID NO: 690 - protein, 12 aa, Artificial Sequence
  synthetic construct
RDENVYECVAQN

SEQ ID NO: 691 - protein, 12 aa, Artificial Sequence
  synthetic construct
DENVYECVAQNS

SEQ ID NO: 692 - protein, 12 aa, Artificial Sequence
  synthetic construct
ENVYECVAQNSV

SEQ ID NO: 693 - protein, 12 aa, Artificial Sequence
  synthetic construct
NVYECVAQNSVG

SEQ ID NO: 694 - protein, 12 aa, Artificial Sequence
  synthetic construct
VYECVAQNSVGE

SEQ ID NO: 695 - protein, 12 aa, Artificial Sequence
  synthetic construct
YECVAQNSVGEI

SEQ ID NO: 696 - protein, 12 aa, Artificial Sequence
  synthetic construct
ECVAQNSVGEIT

SEQ ID NO: 697 - protein, 12 aa, Artificial Sequence
  synthetic construct
CVAQNSVGEITV

SEQ ID NO: 698 - protein, 12 aa, Artificial Sequence
  synthetic construct
VAQNSVGEITVH

SEQ ID NO: 699 - protein, 12 aa, Artificial Sequence
  synthetic construct
AQNSVGEITVHA

SEQ ID NO: 700 - protein, 12 aa, Artificial Sequence
  synthetic construct
QNSVGEITVHAK

SEQ ID NO: 701 - protein, 12 aa, Artificial Sequence
  synthetic construct
NSVGEITVHAKL

SEQ ID NO: 702 - protein, 12 aa, Artificial Sequence
  synthetic construct
SVGEITVHAKLT

SEQ ID NO: 703 - protein, 12 aa, Artificial Sequence
  synthetic construct
VGEITVHAKLTV

SEQ ID NO: 704 - protein, 12 aa, Artificial Sequence
  synthetic construct
GEITVHAKLTVL

SEQ ID NO: 705 - protein, 12 aa, Artificial Sequence
  synthetic construct
EITVHAKLTVLR

SEQ ID NO: 706 - protein, 12 aa, Artificial Sequence
  synthetic construct
ITVHAKLTVLRE

SEQ ID NO: 707 - protein, 13 aa, Artificial Sequence
  synthetic construct
EEPPRFIKEPKDQ

SEQ ID NO: 708 - protein, 13 aa, Artificial Sequence
  synthetic construct
EPPRFIKEPKDQI

SEQ ID NO: 709 - protein, 13 aa, Artificial Sequence
  synthetic construct
PPRFIKEPKDQIG

SEQ ID NO: 710 - protein, 13 aa, Artificial Sequence
  synthetic construct
PRFIKEPKDQIGV

SEQ ID NO: 711 - protein, 13 aa, Artificial Sequence
  synthetic construct
RFIKEPKDQIGVS

SEQ ID NO: 712 - protein, 13 aa, Artificial Sequence
  synthetic construct
FIKEPKDQIGVSG

SEQ ID NO: 713 - protein, 13 aa, Artificial Sequence
  synthetic construct
IKEPKDQIGVSGG

SEQ ID NO: 714 - protein, 13 aa, Artificial Sequence
  synthetic construct
KEPKDQIGVSGGV

SEQ ID NO: 715 - protein, 13 aa, Artificial Sequence
  synthetic construct
EPKDQIGVSGGVA

SEQ ID NO: 716 - protein, 13 aa, Artificial Sequence
  synthetic construct
PKDQIGVSGGVAS

SEQ ID NO: 717 - protein, 13 aa, Artificial Sequence
  synthetic construct
KDQIGVSGGVASF

SEQ ID NO: 718 - protein, 13 aa, Artificial Sequence
  synthetic construct
DQIGVSGGVASFV

SEQ ID NO: 719 - protein, 13 aa, Artificial Sequence
  synthetic construct
QIGVSGGVASFVC

SEQ ID NO: 720 - protein, 13 aa, Artificial Sequence
  synthetic construct
IGVSGGVASFVCQ

SEQ ID NO: 721 - protein, 13 aa, Artificial Sequence
  synthetic construct
GVSGGVASFVCQA

SEQ ID NO: 722 - protein, 13 aa, Artificial Sequence
  synthetic construct
VSGGVASFVCQAT

SEQ ID NO: 723 - protein, 13 aa, Artificial Sequence
  synthetic construct
SGGVASFVCQATG

SEQ ID NO: 724 - protein, 13 aa, Artificial Sequence
  synthetic construct
GGVASFVCQATGD

SEQ ID NO: 725 - protein, 13 aa, Artificial Sequence
  synthetic construct
GVASFVCQATGDP

SEQ ID NO: 726 - protein, 13 aa, Artificial Sequence
  synthetic construct
VASFVCQATGDPK

SEQ ID NO: 727 - protein, 13 aa, Artificial Sequence
  synthetic construct
ASFVCQATGDPKP

SEQ ID NO: 728 - protein, 13 aa, Artificial Sequence
  synthetic construct
SFVCQATGDPKPR

SEQ ID NO: 729 - protein, 13 aa, Artificial Sequence
  synthetic construct
FVCQATGDPKPRV

SEQ ID NO: 730 - protein, 13 aa, Artificial Sequence
  synthetic construct
VCQATGDPKPRVT

SEQ ID NO: 731 - protein, 13 aa, Artificial Sequence
  synthetic construct
CQATGDPKPRVTW

SEQ ID NO: 732 - protein, 13 aa, Artificial Sequence
  synthetic construct
QATGDPKPRVTWN

SEQ ID NO: 733 - protein, 13 aa, Artificial Sequence
  synthetic construct
ATGDPKPRVTWNK

SEQ ID NO: 734 - protein, 13 aa, Artificial Sequence
  synthetic construct
TGDPKPRVTWNKK

SEQ ID NO: 735 - protein, 13 aa, Artificial Sequence
  synthetic construct
GDPKPRVTWNKKG

SEQ ID NO: 736 - protein, 13 aa, Artificial Sequence
  synthetic construct
DPKPRVTWNKKGK

SEQ ID NO: 737 - protein, 13 aa, Artificial Sequence
  synthetic construct
PKPRVTWNKKGKK

SEQ ID NO: 738 - protein, 13 aa, Artificial Sequence
  synthetic construct
KPRVTWNKKGKKV

SEQ ID NO: 739 - protein, 13 aa, Artificial Sequence
  synthetic construct
PRVTWNKKGKKVN

SEQ ID NO: 740 - protein, 13 aa, Artificial Sequence
  synthetic construct
RVTWNKKGKKVNS

SEQ ID NO: 741 - protein, 13 aa, Artificial Sequence
  synthetic construct
VTWNKKGKKVNSQ

SEQ ID NO: 742 - protein, 13 aa, Artificial Sequence
  synthetic construct
TWNKKGKKVNSQR

SEQ ID NO: 743 - protein, 13 aa, Artificial Sequence
  synthetic construct
WNKKGKKVNSQRF

SEQ ID NO: 744 - protein, 13 aa, Artificial Sequence
  synthetic construct
NKKGKKVNSQRFE

SEQ ID NO: 745 - protein, 12 aa, Artificial Sequence
  synthetic construct
KGKKVNSQRFET

SEQ ID NO: 746 - protein, 13 aa, Artificial Sequence
  synthetic construct
KGKKVNSQRFETI

SEQ ID NO: 747 - protein, 13 aa, Artificial Sequence
  synthetic construct
GKKVNSQRFETIE

SEQ ID NO: 748 - protein, 13 aa, Artificial Sequence
  synthetic construct
KKVNSQRFETIEF

SEQ ID NO: 749 - protein, 13 aa, Artificial Sequence
  synthetic construct
KVNSQRFETIEFD

SEQ ID NO: 750 - protein, 13 aa, Artificial Sequence
  synthetic construct
VNSQRFETIEFDE

SEQ ID NO: 751 - protein, 13 aa, Artificial Sequence
  synthetic construct
NSQRFETIEFDES

SEQ ID NO: 752 - protein, 13 aa, Artificial Sequence
  synthetic construct
SQRFETIEFDESA

SEQ ID NO: 753 - protein, 13 aa, Artificial Sequence
  synthetic construct
QRFETIEFDESAG

SEQ ID NO: 754 - protein, 13 aa, Artificial Sequence
  synthetic construct
RFETIEFDESAGA

SEQ ID NO: 755 - protein, 13 aa, Artificial Sequence
  synthetic construct
FETIEFDESAGAV

SEQ ID NO: 756 - protein, 13 aa, Artificial Sequence
  synthetic construct
ETIEFDESAGAVL

SEQ ID NO: 757 - protein, 13 aa, Artificial Sequence
  synthetic construct
TIEFDESAGAVLR

SEQ ID NO: 758 - protein, 13 aa, Artificial Sequence
  synthetic construct
IEFDESAGAVLRI

SEQ ID NO: 759 - protein, 13 aa, Artificial Sequence
  synthetic construct
EFDESAGAVLRIQ

SEQ ID NO: 760 - protein, 13 aa, Artificial Sequence
  synthetic construct
FDESAGAVLRIQP

SEQ ID NO: 761 - protein, 13 aa, Artificial Sequence
  synthetic construct
DESAGAVLRIQPL

SEQ ID NO: 762 - protein, 13 aa, Artificial Sequence
  synthetic construct
ESAGAVLRIQPLR

SEQ ID NO: 763 - protein, 13 aa, Artificial Sequence
  synthetic construct
SAGAVLRIQPLRT

SEQ ID NO: 764 - protein, 13 aa, Artificial Sequence
  synthetic construct
AGAVLRIQPLRTP

SEQ ID NO: 765 - protein, 13 aa, Artificial Sequence
  synthetic construct
GAVLRIQPLRTPR

SEQ ID NO: 766 - protein, 13 aa, Artificial Sequence
  synthetic construct
AVLRIQPLRTPRD

SEQ ID NO: 767 - protein, 13 aa, Artificial Sequence
  synthetic construct
VLRIQPLRTPRDE

SEQ ID NO: 768 - protein, 13 aa, Artificial Sequence
  synthetic construct
LRIQPLRTPRDEN

SEQ ID NO: 769 - protein, 13 aa, Artificial Sequence
  synthetic construct
RIQPLRTPRDENV

SEQ ID NO: 770 - protein, 13 aa, Artificial Sequence
  synthetic construct
IQPLRTPRDENVY

SEQ ID NO: 771 - protein, 13 aa, Artificial Sequence
  synthetic construct
QPLRTPRDENVYE

SEQ ID NO: 772 - protein, 13 aa, Artificial Sequence
  synthetic construct
PLRTPRDENVYEC

SEQ ID NO: 773 - protein, 13 aa, Artificial Sequence
  synthetic construct
LRTPRDENVYECV

SEQ ID NO: 774 - protein, 13 aa, Artificial Sequence
  synthetic construct
RTPRDENVYECVA

SEQ ID NO: 775 - protein, 13 aa, Artificial Sequence
  synthetic construct
TPRDENVYECVAQ

SEQ ID NO: 776 - protein, 13 aa, Artificial Sequence
  synthetic construct
PRDENVYECVAQN

SEQ ID NO: 777 - protein, 13 aa, Artificial Sequence
  synthetic construct
RDENVYECVAQNS

SEQ ID NO: 778 - protein, 13 aa, Artificial Sequence
  synthetic construct
DENVYECVAQNSV

SEQ ID NO: 779 - protein, 13 aa, Artificial Sequence
  synthetic construct
ENVYECVAQNSVG

SEQ ID NO: 780 - protein, 13 aa, Artificial Sequence
  synthetic construct
NVYECVAQNSVGE

SEQ ID NO: 781 - protein, 13 aa, Artificial Sequence
  synthetic construct
VYECVAQNSVGEI

SEQ ID NO: 782 - protein, 13 aa, Artificial Sequence
  synthetic construct
YECVAQNSVGEIT

SEQ ID NO: 783 - protein, 13 aa, Artificial Sequence
  synthetic construct
ECVAQNSVGEITV

SEQ ID NO: 784 - protein, 13 aa, Artificial Sequence
  synthetic construct
CVAQNSVGEITVH

SEQ ID NO: 785 - protein, 13 aa, Artificial Sequence
  synthetic construct
VAQNSVGEITVHA

SEQ ID NO: 786 - protein, 13 aa, Artificial Sequence
  synthetic construct
AQNSVGEITVHAK

SEQ ID NO: 787 - protein, 13 aa, Artificial Sequence
  synthetic construct
QNSVGEITVHAKL

SEQ ID NO: 788 - protein, 13 aa, Artificial Sequence
  synthetic construct
NSVGEITVHAKLT

SEQ ID NO: 789 - protein, 13 aa, Artificial Sequence
  synthetic construct
SVGEITVHAKLTV

SEQ ID NO: 790 - protein, 13 aa, Artificial Sequence
  synthetic construct
VGEITVHAKLTVL

SEQ ID NO: 791 - protein, 13 aa, Artificial Sequence
  synthetic construct
GEITVHAKLTVLR

SEQ ID NO: 792 - protein, 13 aa, Artificial Sequence
  synthetic construct
EITVHAKLTVLRE

SEQ ID NO: 793 - protein, 14 aa, Artificial Sequence
  synthetic construct
EEPPRFIKEPKDQI

SEQ ID NO: 794 - protein, 14 aa, Artificial Sequence
  synthetic construct
EPPRFIKEPKDQIG

SEQ ID NO: 795 - protein, 14 aa, Artificial Sequence
  synthetic construct
PPRFIKEPKDQIGV

SEQ ID NO: 796 - protein, 14 aa, Artificial Sequence
  synthetic construct
PRFIKEPKDQIGVS

SEQ ID NO: 797 - protein, 14 aa, Artificial Sequence
  synthetic construct
RFIKEPKDQIGVSG

SEQ ID NO: 798 - protein, 14 aa, Artificial Sequence
  synthetic construct
FIKEPKDQIGVSGG

SEQ ID NO: 799 - protein, 14 aa, Artificial Sequence
  synthetic construct
IKEPKDQIGVSGGV

SEQ ID NO: 800 - protein, 14 aa, Artificial Sequence
  synthetic construct
KEPKDQIGVSGGVA

SEQ ID NO: 801 - protein, 14 aa, Artificial Sequence
  synthetic construct
EPKDQIGVSGGVAS

SEQ ID NO: 802 - protein, 14 aa, Artificial Sequence
  synthetic construct
PKDQIGVSGGVASF

SEQ ID NO: 803 - protein, 14 aa, Artificial Sequence
  synthetic construct
KDQIGVSGGVASFV

SEQ ID NO: 804 - protein, 14 aa, Artificial Sequence
  synthetic construct
DQIGVSGGVASFVC

SEQ ID NO: 805 - protein, 14 aa, Artificial Sequence
  synthetic construct
QIGVSGGVASFVCQ

SEQ ID NO: 806 - protein, 14 aa, Artificial Sequence
  synthetic construct
IGVSGGVASFVCQA

SEQ ID NO: 807 - protein, 14 aa, Artificial Sequence
  synthetic construct
GVSGGVASFVCQAT

SEQ ID NO: 808 - protein, 14 aa, Artificial Sequence
  synthetic construct
VSGGVASFVCQATG

SEQ ID NO: 809 - protein, 14 aa, Artificial Sequence
  synthetic construct
SGGVASFVCQATGD

SEQ ID NO: 810 - protein, 14 aa, Artificial Sequence
  synthetic construct
GGVASFVCQATGDP

SEQ ID NO: 811 - protein, 14 aa, Artificial Sequence
  synthetic construct
GVASFVCQATGDPK

SEQ ID NO: 812 - protein, 14 aa, Artificial Sequence
  synthetic construct
VASFVCQATGDPKP

SEQ ID NO: 813 - protein, 14 aa, Artificial Sequence
  synthetic construct
ASFVCQATGDPKPR

SEQ ID NO: 814 - protein, 14 aa, Artificial Sequence
  synthetic construct
SFVCQATGDPKPRV

SEQ ID NO: 815 - protein, 14 aa, Artificial Sequence
  synthetic construct
FVCQATGDPKPRVT

SEQ ID NO: 816 - protein, 14 aa, Artificial Sequence
  synthetic construct
VCQATGDPKPRVTW

SEQ ID NO: 817 - protein, 14 aa, Artificial Sequence
  synthetic construct
CQATGDPKPRVTWN

SEQ ID NO: 818 - protein, 14 aa, Artificial Sequence
  synthetic construct
QATGDPKPRVTWNK

SEQ ID NO: 819 - protein, 14 aa, Artificial Sequence
  synthetic construct
ATGDPKPRVTWNKK

SEQ ID NO: 820 - protein, 14 aa, Artificial Sequence
  synthetic construct
TGDPKPRVTWNKKG

SEQ ID NO: 821 - protein, 14 aa, Artificial Sequence
  synthetic construct
GDPKPRVTWNKKGK

SEQ ID NO: 822 - protein, 14 aa, Artificial Sequence
  synthetic construct
DPKPRVTWNKKGKK

SEQ ID NO: 823 - protein, 14 aa, Artificial Sequence
  synthetic construct
PKPRVTWNKKGKKV

SEQ ID NO: 824 - protein, 14 aa, Artificial Sequence
  synthetic construct
KPRVTWNKKGKKVN

SEQ ID NO: 825 - protein, 14 aa, Artificial Sequence
  synthetic construct
PRVTWNKKGKKVNS

SEQ ID NO: 826 - protein, 14 aa, Artificial Sequence
  synthetic construct
RVTWNKKGKKVNSQ

SEQ ID NO: 827 - protein, 14 aa, Artificial Sequence
  synthetic construct
VTWNKKGKKVNSQR

SEQ ID NO: 828 - protein, 14 aa, Artificial Sequence
  synthetic construct
TWNKKGKKVNSQRF

SEQ ID NO: 829 - protein, 14 aa, Artificial Sequence
  synthetic construct
WNKKGKKVNSQRFE

SEQ ID NO: 830 - protein, 14 aa, Artificial Sequence
  synthetic construct
NKKGKKVNSQRFET

SEQ ID NO: 831 - protein, 14 aa, Artificial Sequence
  synthetic construct
KKGKKVNSQRFETI

SEQ ID NO: 832 - protein, 14 aa, Artificial Sequence
  synthetic construct
KGKKVNSQRFETIE

SEQ ID NO: 833 - protein, 14 aa, Artificial Sequence
  synthetic construct
GKKVNSQRFETIEF

SEQ ID NO: 834 - protein, 14 aa, Artificial Sequence
  synthetic construct
KKVNSQRFETIEFD

SEQ ID NO: 835 - protein, 14 aa, Artificial Sequence
  synthetic construct
KVNSQRFETIEFDE

SEQ ID NO: 836 - protein, 14 aa, Artificial Sequence
  synthetic construct
VNSQRFETIEFDES

SEQ ID NO: 837 - protein, 14 aa, Artificial Sequence
  synthetic construct
NSQRFETIEFDESA

SEQ ID NO: 838 - protein, 14 aa, Artificial Sequence
  synthetic construct
SQRFETIEFDESAG

SEQ ID NO: 839 - protein, 14 aa, Artificial Sequence
  synthetic construct
QRFETIEFDESAGA

SEQ ID NO: 840 - protein, 14 aa, Artificial Sequence
  synthetic construct
RFETIEFDESAGAV

SEQ ID NO: 841 - protein, 14 aa, Artificial Sequence
  synthetic construct
FETIEFDESAGAVL

SEQ ID NO: 842 - protein, 14 aa, Artificial Sequence
  synthetic construct
ETIEFDESAGAVLR

SEQ ID NO: 843 - protein, 14 aa, Artificial Sequence
  synthetic construct
TIEFDESAGAVLRI

SEQ ID NO: 844 - protein, 14 aa, Artificial Sequence
  synthetic construct
IEFDESAGAVLRIQ

SEQ ID NO: 845 - protein, 14 aa, Artificial Sequence
  synthetic construct
EFDESAGAVLRIQP

SEQ ID NO: 846 - protein, 14 aa, Artificial Sequence
  synthetic construct
FDESAGAVLRIQPL

SEQ ID NO: 847 - protein, 14 aa, Artificial Sequence
  synthetic construct
DESAGAVLRIQPLR

SEQ ID NO: 848 - protein, 14 aa, Artificial Sequence
  synthetic construct
ESAGAVLRIQPLRT

SEQ ID NO: 849 - protein, 14 aa, Artificial Sequence
  synthetic construct
SAGAVLRIQPLRTP

SEQ ID NO: 850 - protein, 14 aa, Artificial Sequence
  synthetic construct
AGAVLRIQPLRTPR

SEQ ID NO: 851 - protein, 14 aa, Artificial Sequence
  synthetic construct
GAVLRIQPLRTPRD

SEQ ID NO: 852 - protein, 14 aa, Artificial Sequence
  synthetic construct
AVLRIQPLRTPRDE

SEQ ID NO: 853 - protein, 14 aa, Artificial Sequence
  synthetic construct
VLRIQPLRTPRDEN

SEQ ID NO: 854 - protein, 14 aa, Artificial Sequence
  synthetic construct
LRIQPLRTPRDENV

SEQ ID NO: 855 - protein, 14 aa, Artificial Sequence
  synthetic construct
RIQPLRTPRDENVY

SEQ ID NO: 856 - protein, 14 aa, Artificial Sequence
  synthetic construct
IQPLRTPRDENVYE

SEQ ID NO: 857 - protein, 14 aa, Artificial Sequence
  synthetic construct
QPLRTPRDENVYEC

SEQ ID NO: 858 - protein, 14 aa, Artificial Sequence
  synthetic construct
PLRTPRDENVYECV

SEQ ID NO: 859 - protein, 14 aa, Artificial Sequence
  synthetic construct
LRTPRDENVYECVA

SEQ ID NO: 860 - protein, 14 aa, Artificial Sequence
  synthetic construct
RTPRDENVYECVAQ

SEQ ID NO: 861 - protein, 14 aa, Artificial Sequence
  synthetic construct
TPRDENVYECVAQN

SEQ ID NO: 862 - protein, 14 aa, Artificial Sequence
  synthetic construct
PRDENVYECVAQNS

SEQ ID NO: 863 - protein, 14 aa, Artificial Sequence
  synthetic construct
RDENVYECVAQNSV

SEQ ID NO: 864 - protein, 14 aa, Artificial Sequence
  synthetic construct
DENVYECVAQNSVG

SEQ ID NO: 865 - protein, 14 aa, Artificial Sequence
  synthetic construct
ENVYECVAQNSVGE

SEQ ID NO: 866 - protein, 14 aa, Artificial Sequence
  synthetic construct
NVYECVAQNSVGEI

SEQ ID NO: 867 - protein, 14 aa, Artificial Sequence
  synthetic construct
VYECVAQNSVGEIT

SEQ ID NO: 868 - protein, 14 aa, Artificial Sequence
  synthetic construct
YECVAQNSVGEITV

SEQ ID NO: 869 - protein, 14 aa, Artificial Sequence
  synthetic construct
ECVAQNSVGEITVH

SEQ ID NO: 870 - protein, 14 aa, Artificial Sequence
  synthetic construct
CVAQNSVGEITVHA

SEQ ID NO: 871 - protein, 14 aa, Artificial Sequence
  synthetic construct
VAQNSVGEITVHAK

SEQ ID NO: 872 - protein, 14 aa, Artificial Sequence
  synthetic construct
AQNSVGEITVHAKL

SEQ ID NO: 873 - protein, 14 aa, Artificial Sequence
  synthetic construct
QNSVGEITVHAKLT

SEQ ID NO: 874 - protein, 14 aa, Artificial Sequence
  synthetic construct
NSVGEITVHAKLTV

SEQ ID NO: 875 - protein, 14 aa, Artificial Sequence
  synthetic construct
SVGEITVHAKLTVL

SEQ ID NO: 876 - protein, 14 aa, Artificial Sequence
  synthetic construct
VGEITVHAKLTVLR

SEQ ID NO: 877 - protein, 14 aa, Artificial Sequence
  synthetic construct
GEITVHAKLTVLRE

SEQ ID NO: 878 - protein, 9 aa, Artificial Sequence
  synthetic construct
RRRRRRRRR

SEQ ID NO: 879 - protein, 16 aa, Artificial Sequence
  synthetic construct
RQPKIWFPNRRKPWKK

SEQ ID NO: 880 - protein, 11 aa, Artificial Sequence
  synthetic construct
GRKKRRQRPPQ

SEQ ID NO: 881 - protein, 16 aa, Artificial Sequence
  synthetic construct
RQIKIWFQNRRMKWKK

SEQ ID NO: 882 - protein, 16 aa, Artificial Sequence
  synthetic construct
RQIAIWFQNRRMKWAA

SEQ ID NO: 883 - protein, 9 aa, Artificial Sequence
  synthetic construct
RKKRRQRRR

SEQ ID NO: 884 - protein, 21 aa, Artificial Sequence
  synthetic construct
TRSSRAGLQFPVGRVHRLLRK

SEQ ID NO: 885 - protein, 26 aa, Artificial Sequence
  synthetic construct
GWTLNSAGYLLGKINKALAALAKKIL

SEQ ID NO: 886 - protein, 18 aa, Artificial Sequence
  synthetic construct
KLALKLALKALKAALKLA

SEQ ID NO: 887 - protein, 16 aa, Artificial Sequence
  synthetic construct
AAVALLPAVLLALLAP

SEQ ID NO: 888 - protein, 10 aa, Artificial Sequence
  synthetic construct
VPMLKPMLKE

SEQ ID NO: 889 - protein, 28 aa, Artificial Sequence
  synthetic construct
MANLGYWLLALFVTMWTDVGLCKKRPKP

SEQ ID NO: 890 - protein, 18 aa, Artificial Sequence
  synthetic construct
LLIILRRRIRKQAHAHSK

SEQ ID NO: 891 - protein, 21 aa, Artificial Sequence
  synthetic construct
KETWWETWWTEWSQPKKKRKV

SEQ ID NO: 892 - protein, 18 aa, Artificial Sequence
  synthetic construct
RGGRLSYSRRRFSTSTGR

SEQ ID NO: 893 - protein, 15 aa, Artificial Sequence
  synthetic construct
SDLWEMMMVSLACQY

SEQ ID NO: 894 - protein, 12 aa, Artificial Sequence
  synthetic construct
TSPLNIHNGQKL

SEQ ID NO: 895 - protein, 12 aa, Artificial Sequence
  synthetic construct
GRKKRRQRRRPQ

SEQ ID NO: 896 - protein, 10 aa, Artificial Sequence
  synthetic construct
RKKRRQRRRC

SEQ ID NO: 897 - protein, 13 aa, Artificial Sequence
  synthetic construct
ESVEEVVRVPTTA

SEQ ID NO: 898 - protein, 5 aa, Artificial Sequence
  synthetic construct
KKGKK

SEQ ID NO: 899 - protein, 4 aa, Artificial Sequence
  synthetic construct
RTPR

SEQ ID NO: 900 - protein, 24 aa, Artificial Sequence
  synthetic construct
ATERTTSIATTTTTTTESVEEVVR

(19) United States
(12) Patent Application Publication
(10) Pub. No.: US 2022/0072097 A1
(43) Pub. Date: Mar. 10, 2022

(54) PEPTIDES AND METHODS FOR TREATING NEURODEGENERATIVE DISORDERS

(71) Applicant: OHIO STATE INNOVATION FOUNDATION, Columbus, OH (US)

(72) Inventors: Yingjie SHEN, Columbus, OH (US); Yuanzheng GU, Bedford, MA (US); Kui XU, Cincinnati, OH (US)

(21) Appl. No.: 17/482,750

(22) Filed: Sep. 23, 2021

Related U.S. Application Data
(63) Continuation of application No. 16/300,687, filed on Nov. 12, 2018.
(60) Provisional application No. 62/335,159, filed on May 12, 2016.

(51) Int. Cl. A61K 38/17; C07K 14/47; C07K 7/08; A61P 25/28; A61K 38/10; A61K 38/12; A61K 38/51

(52) U.S. Cl. CPC A61K 38/1716; A61K 38/177; C07K 14/4703; C07K 7/08; C12Y 402/02004; A61K 38/10; A61K 38/12; A61K 38/51; A61P 25/28

(57) ABSTRACT

Disclosed herein are compositions and methods for treating and preventing neurodegenerative diseases, such as Alzheimer's disease. In some embodiments, the composition comprises a peptide that disrupts the binding between PTPσ and APP, preventing β-amyloidogenic processing of APP without affecting other major substrates of β- and γ-secretases. Alternatively, in some embodiments, an antibody or a fragment of an antibody against PTPσ or APP may be used to disrupt the binding between PTPσ and APP. In some embodiments, the composition comprises compounds or enzymes, which restore perineuronal balance of PTPσ ligands CS and HS, thereby preventing abnormally increased β-amyloidogenic processing of APP. Compositions and methods disclosed herein can be used in combination to treat and prevent neurodegenerative diseases.

### CROSS REFERENCE TO RELATED APPLICATIONS

This application is a continuation of U.S. application Ser. No. 16/300,687, filed Nov. 12, 2018, which is a national stage application filed under 35 U.S.C. § 371 of PCT/US2017/032387 filed May 12, 2017, which claims the benefit of U.S. Provisional Application No. 62/335,159, filed May 12, 2016, which are hereby incorporated by reference in their entirety for all purposes.

### STATEMENT REGARDING SEQUENCE LISTING

Applicant submits herewith a Sequence Listing in computer readable form and in compliance with 37 C.F.R. §§ 1.821-1.825. This sequence listing is in ASCII TXT format with filename “10336-185US2_2021_09_23_Sequence_Listing,” a 200,001 bytes file size, and creation date of May 12, 2017. The content of the Sequence Listing is hereby incorporated by reference.

### BACKGROUND

Alzheimer's disease (AD) is the most common form of dementia, and its risk accelerates after age 65. With a rapidly expanding aging population, AD is projected to become an overwhelming medical burden to the world.

A definitive pathological hallmark of Alzheimer's disease (AD) is the progressive aggregation of β-amyloid (Aβ) peptides in the brain, a process also known as β-amyloidosis, which is often accompanied by neuroinflammation and formation of neurofibrillary tangles containing Tau, a microtubule binding protein_¹.

Evidence from human genetic studies showed that overproduction of Aβ due to gene mutations inevitably inflicts cascades of cytotoxic events, ultimately leading to neurodegeneration and decay of brain functions. Cerebral accumulation of Aβ peptides, especially in their soluble forms, is therefore recognized as a key culprit in the development of AD¹. In the brain, Aβ peptides mainly derive from sequential cleavage of neuronal Amyloid Precursor Protein (APP) by the β- and γ-secretases. However, despite decades of research, molecular regulation of the amyloidogenic secretase activities remains poorly understood, hindering the design of therapeutics to specifically target the APP amyloidogenic pathway.

Pharmacological inhibition of the β- and γ-secretase activities, although effective in suppressing Aβ production, interferes with physiological function of the secretases on their other substrates. Such intervention strategies therefore are often innately associated with untoward side effects, which have led to several failed clinical trials in the past²⁻⁴. To date, no therapeutic regimen is available to prevent the onset of AD or curtail its progression.

Besides Aβ, Tau is another biomarker that has been intensively studied in AD. Cognitive decline in patients sometimes correlates better with Tau pathology than with Aβ burden^{5,6}. Overwhelming evidence also substantiated that malfunction of Tau contributes to synaptic loss and neuronal deterioration⁷.

In addition to AD, many other neurodegenerative diseases also involves Aβ or Tau pathologies, and there is no disease modifying therapy available for any of these debilitating diseases.

### SUMMARY

Disclosed herein are peptides, compositions, and methods to treat and prevent neurodegenerative diseases that involve β-amyloid pathologies and/or Tau pathologies, including but not limited to Alzheimer's disease, Lewy body dementia, frontotemporal dementia, cerebral amyloid angiopathy, primary age-related tauopathy, chronic traumatic encephalopathy, Parkinson's disease, postencephalitic parkinsonism, Huntington's disease, amyolateral sclerosis, Pick's disease, progressive supranuclear palsy, corticobasal degeneration, Lytico-Bodig disease, ganglioglioma and gangliocytoma, subacute sclerosing panencephalitis, Hallervorden-Spatz disease, and/or Creutzfeldt-Jakob disease.

These peptides, compositions, and methods may also be used to prevent these neurodegenerative diseases in at-risk subjects, such as people with Down syndrome and those who have suffered from brain injuries or cerebral ischemia, as well as the aging population.

In some embodiments, the disclosed peptides, compositions, and methods disrupt the binding between Protein Tyrosine Phosphatase sigma (PTPσ) and APP, preventing β-amyloidogenic processing of APP as well as Tau aggregation.

In some embodiments, the disclosed compositions and methods restore the physiological balance of two classes of PTPσ ligands in the brain microenvironment, namely the chondroitin sulfates (CS) and heparin or its analog heparan sulfates (HS), and thereby prevent abnormally increased β-amyloidogenic processing of APP.

Unlike the anti-Aβ antibodies in current clinical trials that passively clear β-amyloid, the therapeutic strategy disclosed herein inhibits the process upstream of β-amyloid production. Unlike the β- and γ-secretase inhibitors in current clinical trials, the therapeutic strategy disclosed herein inhibits β-amyloid production without affecting other major substrates of these secretases. Therefore the strategy disclosed herein may be more effective with fewer side effects compared to the most advanced AD drug candidates in clinical trials.

Disclosed herein is a peptide for treating or preventing the aforementioned neurodegenerative disorders, the peptide comprising a decoy fragment of APP, a decoy fragment of PTPσ, or a combination thereof. In some embodiments, the decoy fragment of APP is a peptide comprising at least 5 consecutive amino acids of SEQ ID NO:1. In some embodiments, the decoy fragment of APP is a peptide comprising at least 10 consecutive amino acids of SEQ ID NO:1. For example, the decoy fragment of APP can comprise an amino acid sequence selected from the group consisting of SEQ ID NO:88, SEQ ID NO:91, SEQ ID NO:101, SEQ ID NO:112, SEQ ID NO:139, SEQ ID NO:151, SEQ ID NO:157, SEQ ID NO:251, SEQ ID NO:897. In some embodiments, the decoy fragment of PTPσ is a peptide comprising at least 4 consecutive amino acids of SEQ ID NO:442. For example, the decoy fragment of PTPσ can comprises the amino acid sequence SEQ ID NO:655, SEQ ID NO:769, SEQ ID NO:898, or SEQ ID NO:899. In some embodiments, the peptide further comprises a blood brain barrier penetrating sequence. For example, the blood brain barrier penetrating sequence comprises amino acid sequence SEQ ID NO: 880, SEQ ID NO: 883, SEQ ID NO: 888, SEQ ID NO: 894, SEQ ID NO: 895, SEQ ID NO: 896.

Also disclosed is a method that restores the physiological molecular CS/HS balance that may be used to treat and prevent aforementioned neurodegenerative diseases. In some embodiments, administering HS, or its analog heparin, or their mimetics modified to reduce anti-coagulant effect, with a saccharide chain length of 17, 18, 19, 20, 21, 22, 23, 24 units or longer, could assist in restoring the CS/HS balance. In some embodiments, the physiological molecular CS/HS balance is restored by administering enzymes that digest CS (such as Chondroitinase ABC, also known as ChABC) or prevent HS degradation (such as Heparanase inhibitors PI-88, OGT 2115, or PG545). Alternatively or in addition, agents that mimic the HS/heparin effect of PTPσ clustering⁸, such as multivalent antibodies, could be administered.

Also disclosed is a method of treating a neurodegenerative disorder in a subject, the method comprising administering to the subject an aforementioned composition or combination of compositions. In some embodiments, the neurodegenerative disease is selected from the group consisting of Alzheimer's Disease, Lewy body dementia, frontotemporal dementia, cerebral amyloid angiopathy, primary age-related tauopathy, chronic traumatic encephalopathy, Parkinson's disease, postencephalitic parkinsonism, Huntington's disease, amyolateral sclerosis, Pick's disease, progressive supranuclear palsy, corticobasal degeneration, Lytico-Bodig disease, ganglioglioma and gangliocytoma, subacute sclerosing panencephalitis, Hallervorden-Spatz disease, and/or Creutzfeldt-Jakob disease. In some embodiments, subjects are selected from at-risk populations, such as the aging population, people with Down syndrome, and those suffered from brain injuries or cerebral ischemia, to prevent subsequent onset of neurodegenerative diseases.

Also disclosed is a method of screening for candidate compounds that slow, stop, reverse, or prevent neurodegeneration. In some embodiments, the method comprises providing a sample comprising APP and PTPσ in an environment permissive for APP-PTPσ binding, contacting the sample with a candidate compound, and assaying the sample for APP-PTPσ binding, wherein a decrease in APP-PTPσ binding compared to control values is an indication that the candidate agent is effective to slow, stop, reverse, or prevent neurodegeneration. In some embodiments, the method comprises contacting/incubating a candidate compound with cell membrane preparations extracted from fresh rodent brain homogenates, wherein a decrease in APP β- and/or γ-cleavage products is an indication that the candidate agent has the potential to slow, stop, reverse, or prevent neurodegeneration.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A-1I. PTPσ is an APP binding partner in the brain. a-f, Colocalization of PTPσ (a, green) and APP (b, red) in hippocampal CA1 neurons of adult rat is shown by confocal imaging. Nuclei of CA1 neurons are stained with DAPI (c, blue). d, Merge of three channels. Scale bar, 50 μm. e, Zoom-in image of the soma layer in d. Arrows, intensive colocalization of PTPσ and APP in the initial segments of apical dendrites; arrow heads, punctates of colocalization in the perinuclear regions. Scale bar, 20 μm. f, Zoom-in image of the very fine grained punctates in the axonal compartment in d. Arrows points to the colocalization of PTPσ and APP in axons projecting perpendicular to the focal plane. Scale bar, 10 μm. g, Schematic diagram of PTPσ expressed on cell surface as a two-subunit complex. PTPσ is post-translationally processed into an extracellular domain (ECD) and a transmembrane-intracellular domain (ICD). These two subunits associate with each other through noncovalent bond. Ig-like, immunoglobulin-like domains; FNIII-like, fibronectin III-like domains; D1 and D2, two phosphatase domains. h, i, Co-immunoprecipitation (co-IP) of PTPσ and APP from mouse forebrain lysates. Left panels, expression of PTPσ and APP in mouse forebrains. Right panels, IP using an antibody specific for the C-terminus (C-term) of APP. Full length APP (APP FL) is detected by anti-APP C-term antibody. h, PTPσ co-IP with APP from forebrain lysates of wild type but not PTPσ-deficient mice (Balb/c background), detected by an antibody against PTPσ-ECD. i, PTPσ co-IP with APP from forebrain lysates of wild type but not APP knockout mice (B6 background), detected by an antibody against PTPσ-ICD. Dotted lines in i indicate lanes on the same western blot exposure that were moved adjacent to each other. Images shown are representatives of at least three independent experiments using mice between ages of 1 month to 2 years.

FIGS. 2A-2C. Molecular complex of PTPσ and APP in brains of various rodent species. a, b, Co-immunoprecipitation using an anti-APP antibody specific for amino acid residues 1-16 of mouse Aβ (clone M3.2). PTPσ and APP binding interaction is detected in forebrains of Balb/c (a) and B6 (b) mice. c, PTPσ co-immunoprecipitates with APP from rat forebrain lysates using an antibody specific for the C-terminus of APP. Images shown are representatives of at least three independent experiments using different animals.

FIGS. 3A-3I. Genetic depletion of PTPσ reduces β-amyloidogenic products of APP. a, Schematic diagram showing amyloidogenic processing of APP by the β- and γ-secretases. Full length APP (APP FL) is cleaved by β-secretase into soluble N-terminal (sAPPβ) and C-terminal (CTFβ) fragments. APP CTFβ can be further processed by γ-secretase into a C-terminal intracellular domain (AICD) and an Aβ peptide. Aggregation of Aβ is a definitive pathology hallmark of AD. b, PTPσ deficiency reduces the level of an APP CTF at about 15 KD in mouse forebrain lysates, without affecting the expression of APP FL. Antibody against the C-terminus of APP recognizes APP FL and CTFs of both mouse and human origins. c and d, The 15 KD APP CTF is identified as CTFβ by immunoprecipitation (IP) followed with western blot analysis, using a pair of antibodies as marked in the diagram (a). Antibodies against amino acids 1-16 of Aβ (anti-Aβ1-16) detect CTFβ but not CTFα, as the epitope is absent in CTFα. c, Mouse endogenous CTFβ level is reduced in PTPσ-deficient mouse brains. 4 repeated experiments were quantified by densitometry. d, Human transgenic CTFβ level is reduced in PTPσ-deficient mouse brains harboring human APP-SwDI transgene. 6 repeated experiments were quantified by densitometry. Within each experiment in both c and d, the value from PTPσ deficient sample was normalized to that from the sample with wild type PTPσ. e and f, PTPσ deficiency reduces the levels of A1340 (e) and A1342 (f) in TgAPP-SwDI mice as measured by ELISA assays. n=12 for each group. The mean values from PTPσ deficient samples was normalized to that from the samples with wild type PTPσ. g and h, Aβ deposition in the hippocampus of 10-month old TgAPP-SwDI mice. Images shown are representatives of 5 pairs of age- and sex-matched mice between 9- to 11-month old. Aβ (green) is detected by immunofluorescent staining using anti-Aβ antibodies clone 6E10 (g) and clone 4G8 (h). DAPI staining is shown in blue. PTPσ deficiency significantly decreases Aβ burden in the brains of TgAPP-SwDI mice. h, Upper panels, the stratum oriens layer between dorsal subiculum (DS) and CA1 (also shown with arrows in g); middle panels, oriens layer between CA1 and CA2; lower panels, the hilus of dentate gyrus (DG, also shown with arrow heads in g). Left column, control staining without primary antibody (no 1° Ab). No Aβ signal is detected in non-transgenic mice (data not shown). Scale bars, 500 μm in g and 100 μm in h. i, Genetic depletion of PTPσ suppresses the progression of Aβ pathology in TgAPP-SwDI mice. ImageJ quantification of Aβ immunofluorescent staining (with 6E10) in DG hilus from 9- and 16-month old TgAPP-SwDI mice. n=3 for each group. Total integrated density of Aβ in DG hilus was normalized to the area size of the hilus to yield the average intensity as show in the bar graph. Mean value of each group was normalized to that of 16 month old TgAPP-SwDI mice expressing wild type PTPσ. All p values, Student's t test, 2-tailed. Error bars, SEM.

FIGS. 4A-4F. Genetic depletion of PTPσ reduces β-amyloidogenic products of APP. a and b, Antibody against the C-terminus of APP recognizes full length (FL) and C-terminal fragments (CTFs) of both mouse and human APP. PTPσ deficiency does not affect the expression level of APP FL (a), but reduces the level of an APP CTF at about 15 KD in mouse forebrain lysates (b). Images shown are representatives of at least three independent experiments. c, Human CTFβ in the forebrains of APP-SwInd transgenic mice is identified using the method as described in FIG. 2d. CTFβ is immunoprecipitated by an antibody against the C-terminus of APP and detected by western blot analysis using an antibody against amino acids 1-16 of human Aβ (6E10), which reacts with CTFβ but not CTFα (regions of antibody epitopes are shown in FIG. 2a). d, Densitometry quantification of experiments as shown in panel c repeated with 5 pairs of mice. For each experiment, the value from PTPσ deficient sample was normalized to the value from the sample with wild type PTPσ. e, Representative images of Aβ immunofluorescent staining (with 6E10) in the hippocampus of 15-month old TgAPP-SwInd mice. Arrows point to Aβ deposits. Scale bars, 50 μm. f, immunofluorescent staining in the hippocampus of 15-month old TgAPP-SwInd mice, as shown in panel e, was quantified using ImageJ. APP-SwInd(+)PTPσ(+/+), n=7; APP-SwInd(+)PTPσ(−/−), n=8. The mean value of APP-SwInd(+)PTPσ(−/−) samples was normalized to that of APP-SwInd(+)PTPσ(+/+) samples. All error bars, SEM. All p values, Student's t test, 2-tailed.

FIGS. 5A-5C. Lower affinity between BACE1 and APP in PTPσ-deficient brains. a, Co-immunoprecipitation experiments show nearly equal BACE1-APP association in wild type and PTPσ-deficient mouse brains under mild detergent condition (1% NP40). However, in PTPσ-deficient brains, BACE1-APP association detected by co-immunoprecipitation is more vulnerable to increased detergent stringency as compared to that in wild type brains. Panels of blots show full length APP (APP FL) pulled down with an anti-BACE1 antibody from mouse forebrain lysates. NP40, Nonidet P-40, non-ionic detergent. SDS, Sodium dodecyl sulfate, ionic detergent. b, Co-immunoprecipitation under buffer condition with 1% NP40 and 0.3% SDS, as shown in the middle panel of a, were repeated with three pair of mice. Each experiment was quantified by densitometry, and the value from PTPσ-deficient sample was calculated as a percentage of that from the wild type sample (also shown as orange points in c). Error bar, SEM. p value, Student's t test, 2-tailed. c, Co-immunoprecipitation experiments were repeated under each detergent condition. The percentage values shown in dots are derived using the same method as in b. Bars represent means. Increasingly stringent buffer conditions manifest a lower BACE1-APP affinity in PTPσ-deficient brains. p value and R², linear regression.

FIGS. 6A-6F. PTPσ does not generically modulate b- and g-secretases. Neither expression levels of the secretases or their activities on other major substrates are affected by PTPσ depletion. Mouse forebrain lysates with or without PTPσ were analyzed by western blot. a and b, PTPσ deficiency does not change expression level of BACE1 (a) or γ-secretase subunits (b). Presenilin1 and 2 (PS1/2) are the catalytic subunits of γ-secretase, which are processed into N-terminal and C-terminal fragments (NTF and CTF) in their mature forms. Nicastrin, Presenilin Enhancer 2 (PEN2), and APH1 are other essential subunits of γ-secretase. c, PTPσ deficiency does not change the level of Neuregulin1 (NGR1) CTFβ, the C-terminal cleavage product by BACE1. NRG1 FL, full length Neuregulin1. d, The level of Notch cleavage product by γ-secretase is not affected by PTPσ deficiency. TMIC, Notch transmembrane/intracellular fragment, which can be cleaved by γ-secretase into a C-terminal intracellular domain NICD (detected by an antibody against Notch C-terminus in the upper panel, and by an antibody specific for γ-secretase cleaved NICD in the lower panel). e, Actin loading control for a and c. f, Actin loading control for b and d. All images shown are representatives of at least three independent experiments. All images shown are representatives of at least three independent experiments using different animals.

FIGS. 7A-7K. PTPσ deficiency attenuates reactive astrogliosis in APP transgenic mice. Expression level of GFAP, a marker of reactive astrocytes, is suppressed in the brains of TgAPP-SwDI mice by PTPσ depletion. Representative images show GFAP (red) and DAPI staining of nuclei (blue) in the brains of 9-month old TgAPP-SwDI mice with or without PTPσ, along with their non-transgenic wild type littermate. a-f, Dentate gyrus (DG) of the hippocampus; scale bars, 100 μm. g-j, Primary somatosensory cortex; scale bars, 200 μm. k, ImageJ quantification of GFAP level in DG hilus from TgAPP-SwDI mice aged between 9 to 11 months. APP-SwDI(−)PTPσ(+/+), non-transgenic wild type littermates (expressing PTPσ but not the human APP transgene). Total integrated density of GFAP in DG hilus was normalized to the area size of the hilus to yield average intensity as shown in the bar graph. Mean value of each group was normalized to that of APP-SwDI(−)PTPσ(+/+) mice. APP-SwDI(−)PTPσ(+/+), n=4; APP-SwDI(+)PTPσ(+/+), n=4; APP-SwDI(+)PTPσ(−/−), n=6. All p values, Student's t test, 2-tailed. Error bars, SEM.

FIGS. 8A-8G. PTPσ deficiency protects APP transgenic mice from synaptic loss. Representative images show immunofluorescent staining of presynaptic marker Synaptophysin in the mossy fiber terminal zone of CA3 region. a-f, Synaptophysin, red; DAPI, blue. Scale bars, 100 μm. g, ImageJ quantification of Synaptophysin expression level in CA3 mossy fiber terminal zone from mice aged between 9 to 11 months. Total integrated density of Synaptophysin in CA3 mossy fiber terminal zone was normalized to the area size to yield average intensity as shown in the bar graph. Mean value of each group was normalized to that of wild type APP-SwDI(−) PTPσ(+/+) mice. APP-SwDI(−)PTPσ(+/+), n=4; APP-SwDI(+)PTPσ(+/+), n=6; APP-SwDI(+)PTPσ(−/−), n=6. All p values, Student's t test, 2-tailed. Error bars, SEM.

FIGS. 9A-9H. PTPσ deficiency mitigates Tau pathology in TgAPP-SwDI mice. a, Schematic diagram depicting distribution pattern of Tau aggregation (green) detected by immunofluorescent staining using an anti-Tau antibody (Tau-5) against its proline-rich region, in brains of 9 to 11 month-old TgAPP-SwDI transgenic mice. Similar results are seen with Tau-46, an antibody recognizing the C-terminus of Tau (Extended Data FIG. 6). Aggregated Tau is found most prominently in the molecular layer of piriform and entorhinal cortex, and occasionally in hippocampal regions in APP-SwDI(+)PTPσ(+/+) mice. b, PTPσ deficiency diminishes Tau aggregation. Bar graph shows quantification of Tau aggregation in coronal brain sections from 4 pairs of age- and sex-matched APP-SwDI(+)PTPσ(+/+) and APP-SwDI(+)PTPσ(−/−) mice of 9 to 11 month-old. For each pair, the value from APP-SwDI(+)PTPσ(−/−) sample is normalized to the value from APP-SwDI(+)PTPσ(+/+) sample. p value, Student's t test, 2-tailed. Error bar, SEM. c, d, Representative images of many areas with Tau aggregation in APP-SwDI(+)PTPσ(+/+) brains. f, g, Representative images of a few areas with Tau aggregation in age-matched APP-SwDI(+)PTPσ(−/−) brains. c and f, Hippocampal regions. d-h, Piriform cortex. e, Staining of a section adjacent to d, but without primary antibody (no 1° Ab). h, no Tau aggregates are detected in aged-matched non-transgenic wild type littermates (expressing PTPσ but not the human APP transgene). Tau, green; DAPI, blue. Arrows points to Tau aggregates. Scale bars, 50 μm.

FIGS. 10A-10E. PTPσ deficiency mitigates Tau pathology in TgAPP-SwInd mice. Tau aggregation (green) is detected by immunofluorescent staining, using an anti-Tau antibody (Tau-5, as in FIG. 5) in the brains of 15 month-old TgAPP-SwInd transgenic mice. Similar results are seen with Tau-46, an antibody recognizing the C-terminus of Tau (Extended Data FIG. 6). Aggregated Tau is found most prominently in the molecular layer of the entorhrinal (a, b) and piriform cortex (c, d), and occasionally in the hippocampal regions (images not shown). e, PTPσ deficiency diminishes Tau aggregation as quantified in coronal brain sections from 15 month-old APP-SwInd(+)PTPσ(+/+) (n=7) and APP-SwInd(+)PTPσ(−/−) mice (n=8). The mean value of APP-SwInd(+)PTPσ(−/−) samples is normalized to that of APP-SwInd(+)PTPσ(+/+). p value, Student's t test, 2-tailed. Error bars, SEM. Tau, green; DAPI, blue. Arrows points to Tau aggregates. Scale bars, 50 μm.

FIGS. 11A-11J. Morphology of Tau aggregates found in APP transgenic brains. a-h, Tau aggregation (green) is detected by immunofluorescent staining, using an anti-Tau antibody (Tau-5) against the proline-rich domain of Tau (same as in FIG. 5 and Extended Data FIG. 5). Tau aggregates in TgAPP-SwDI and TgAPP-SwInd brains show similar morphologies. a-f, Many of the Tau aggregates are found in punctate shapes, likely as part of cell debris, in areas that are free of nuclei staining. g, h, Occasionally the aggregates are found in fibrillary structures, probably in degenerated cells before disassembling. i, An additional anti-Tau antibody (Tau-46), which recognizes the C-terminus of Tau, detects Tau aggregation in the same pattern as Tau-5. j, Image of staining without primary antibody at the same location of the Tau aggregates in the section adjacent to i. Both these antibodies recognize Tau regardless of its phosphorylation status. Tau, green; DAPI, blue. All scale bars, 20 μm.

FIG. 12. Tau expression is not affected by PTPσ or human APP transgenes. Upper panel, total Tau level in brain homogenates. Lower panel, Actin as loading control. Tau protein expression level is not changed by genetic depletion of PTPσ or expression of mutated human APP transgenes. All mice are older than 1 year, and mice in each pair are age- and sex matched. Images shown are representatives of three independent experiments.

FIGS. 13A-13C. PTPσ deficiency rescues behavioral deficits in TgAPP-SwDI mice. a, In the Y-maze assay, performance of spatial navigation is scored by the percentage of spontaneous alternations among total arm entries. Values are normalized to that of non-transgenic wild type APP-SwDI(−)PTPσ(+/+) mice within the colony. Compared to non-transgenic wild type mice, APP-SwDI(+)PTPσ(+/+) mice show deficit of short-term spatial memory, which is rescued by genetic depletion of PTPσ in APP-SwDI(+)PTPσ(−/−) mice. APP-SwDI(−)PTPσ(+/+), n=23 (18 females and 5 males); APP-SwDI(+)PTPσ(+/+), n=52 (30 females and 22 males); APP-SwDI(+)PTPσ(−/−), n=35 (22 females and 13 males). Ages of all genotype groups are similarly distributed between 4 and 11 months. b, c, Novel object test. NO, novel object. FO, familiar object. Attention to NO is measured by the ratio of NO exploration to total object exploration (NO+FO) in terms of exploration time (b) and visiting frequency (c). Values are normalized to that of non-transgenic wild type mice. APP-SwDI(+)PTPσ(+/+) mice showed decreased interest in NO compared to wild type APP-SwDI(−)PTPσ(+/+) mice. The deficit is reversed by PTPσ depletion in APP-SwDI(+)PTPσ(−/−) mice. APP-SwDI(−)PTPσ(+/+), n=28 (19 females and 9 males); APP-SwDI(+)PTPσ(+/+), n=46 (32 females and 14 males); APP-SwDI(+)PTPσ(−/−), n=29 (21 females and 8 males). Ages of all groups are similarly distributed between 4 and 11 months. All p values, Student's t test, 2-tailed. Error bars, SEM.

FIG. 14. PTPσ deficiency restores short-term spatial memory in TgAPP-SwDI mice. In the Y-maze assay, performance of spatial navigation is scored by the percentage of spontaneous alternations among total arm entries. The raw values shown here are before normalization in FIG. 6a. Compared to non-transgenic wild type APP-SwDI(−)PTPσ(+/+)mice, APP-SwDI(+)PTPσ(+/+) mice show deficit of short-term spatial memory, which is rescued by genetic depletion of PTPσ. APP-SwDI(−)PTPσ(+/+), n=23 (18 females and 5 males); APP-SwDI(+)PTPσ(+/+), n=52 (30 females and 22 males); APP-SwDI(+)PTPσ(−/−), n=35 (22 females and 13 males). Ages of all genotype groups are similarly distributed between 4 and 11 months. All p values, Student's t test, 2-tailed. Error bars, SEM.

FIGS. 15A-15D. PTPσ deficiency enhances novelty exploration by TgAPP-SwDI mice. NO, novel object. FO, familiar object. a and b, In novel object test, NO preference is measured by the ratio between NO and FO exploration, where NO/FO>1 indicates preference for NO. c and d, Attention to NO is additionally measured by the discrimination index, NO/(NO+FO), the ratio of NO exploration to total object exploration (NO+FO). The raw values shown here in c and d are before normalization in FIGS. 6b and c. Mice of this colony show a low baseline of the NO/(NO+FO) discrimination index, likely inherited from their parental Balb/c line. For non-transgenic wild type APP-SwDI(−)PTPσ(+/+) mice, the discrimination index is slightly above 0.5 (chance value), similar to what was previously reported for the Balb/c wild type mice²⁷. Thus, a sole measurement of the discrimination index may not reveal the preference for NO as does the NO/FO ratio. Although not as sensitive in measuring object preference, the NO/(NO+FO) index is most commonly used as it provides a normalization of the NO exploration to total object exploration activity. While each has its own advantage and shortcoming, both NO/FO and NO/NO+FO measurements consistently show that the expression of TgAPP-SwDI gene leads to a deficit in attention to the NO, whereas genetic depletion of PTPσ restores novelty exploration to a level close to that of non-transgenic wild type mice. a and c, measurements in terms of exploration time. b and d, measurements in terms of visiting frequency. APP-SwDI(−)PTPσ(+/+), n=28 (19 females and 9 males); APP-SwDI(+)PTPσ(+/+), n=46 (32 females and 14 males); APP-SwDI(+)PTPσ(−/−), n=29 (21 females and 8 males). Ages of all groups are similarly distributed between 4 and 11 months. All p values, Student's t test, 2-tailed. Error bars, SEM.

FIGS. 16A-16C. PTPσ deficiency improves behavioral performance of TgAPP-SwInd mice. a, Performance of spatial navigation is scored by the percentage of spontaneous alternations among total arm entries in the Y-maze assay. Compared to APP-SwInd(+)PTPσ(+/+) mice, APP-SwInd(+)PTPσ(−/−) mice showed improved short-term spatial memory. APP-SwInd(+)PTPσ(+/+), n=40 (20 females and 20 males); APP-SwInd(+)PTPσ(−/−), n=18 (9 females and 9 males). Ages of both genotype groups are similarly distributed between 4 and 11 months. b, c, Novel object test. NO, novel object. FO, familiar object. NO preference is measured by the ratio of NO exploration time to total object exploration time (b) and the ratio of NO exploration time to FO exploration time (c). PTPσ depletion significantly improves novelty preference in these transgenic mice. APP-SwInd(+)PTPσ(+/+), n=43 (21 females and 22 males); APP-SwInd(+)PTPσ(−/−), n=24 (10 females and 14 males). Ages of both groups are similarly distributed between 5 and 15 months. All p values, Student's t test, 2-tailed. Error bars, SEM.

FIG. 17. CS and HS regulate β-cleavage of APP in opposite manners. Membrane preparations from fresh mouse brain homogenates are incubated with CS18 (chondroitin sulfate of 18 oligosaccharides) or HS17 (heparan sulfate analog, heparin fragment of 17 oligosaccharides) at 37° C. for 30 min. Levels of APP β-cleavage product (CTFβ) as detected by Western blot analysis are enhanced by CS18 treatment but diminished by HS17 treatment. FL APP, full length APP. Control, no treatment.

FIGS. 18A and 18B. TBI enhances PTPσ-APP binding and β-cleavage of APP. a, Co-immunoprecipitation of PTPσ with APP showed increased PTPσ-APP binding in after TBI in rat. b, Level of APP β-cleavage product (CTFβ) is enhanced in correlation with increased PTPσ-APP binding. Similar results are found using in mouse TBI brains.

FIG. 19 Heparin fragment of 17 oligosaccharides inhibits APP-PTPσ binding. Recombinant human APP fragment binding to PTPσ is detected by kinetic ELISA assay. Heparin fragment of 17 oligosaccharides (heparan sulfate analog) effectively disrupts APP-PTPσ binding when included in the binding assay. APP fragment used here corresponds to SEQ ID NO:1, which is the region between E1 and E2 domains. PTPσ fragment used here includes its IG1 and IG2 domains.

FIG. 20 Ligand binding site of PTPσ IG1 domain interacts with APP. Binding of human APP fragment (SEQ ID NO:1) with various PTPσ fragments is measured by kinetic ELISA assay. APP fragment corresponds to SEQ ID NO:1, which is a region between E1 and E2 domains. PTPσ fragments used here include IG1,2 (containing IG1 and IG2 domains), ΔLysIG1,2 (containing IG1 and IG2 domains, with lysine 67, 68, 70, 71 mutated to alanine), IG1-FN1 (containing IG1, IG2, IG3 and FN1 domains), ECD (full extracellular domain of PTPσ containing all 3 IG domains and 4 FN domains). Value shown are mean±SEM, n=3 for each group. ***, p≤0.001, Student t test, comparison with the IG1,2.

### DETAILED DESCRIPTION

Experimental results in Example 1 show that neuronal receptor PTPσ mediates both β-amyloid and Tau pathogenesis in two mouse models. In the brain, PTPσ binds to APP. Depletion of PTPσ reduces the affinity between APP and β-secretase, diminishing APP proteolytic products by β- and γ-cleavage without affecting other major substrates of the secretases, suggesting a specificity of β-amyloidogenic regulation. In human APP transgenic mice during aging, the progression of β-amyloidosis, Tau aggregation, neuroinflammation, synaptic loss, as well as behavioral deficits, all show unambiguous dependency on the expression of PTPσ. Additionally, the aggregates of endogenous Tau are found in a distribution pattern similar to that of early stage neurofibrillary tangles in Alzheimer brains. Together, these findings unveil a gatekeeping role of PTPσ upstream of the degenerative pathogenesis, indicating a potential for this neuronal receptor as a drug target for Alzheimer's disease.

Experimental results in Example 2 show that two classes of PTPσ ligands in the brain microenvironment, CS and HS, regulate APP amyloidogenic processing in opposite manners. CS increases APP n-cleavage products, whereas HS decreases APP n-cleavage products. Because CS and HS compete to interact with receptor PTPσ yet lead to opposite signaling and neuronal responses, the ratio of perineuronal CS and HS is therefore crucial for the downstream effects of PTPσ and maintaining the health of the brain.

Experimental results in Example 3 further define that the binding between APP and PTPσ is mediated by a fragment on APP between its E1 and E2 domain and the IG1 domain of PTPσ.

The findings that PTPσ plays a pivotal role in the development of β-amyloid and Tau pathologies indicate that peptides, compositions, and methods disclosed herein may be suitable to treat and prevent neurodegenerative diseases that involve β-amyloid pathologies and/or Tau pathologies, including but not limited to Alzheimer's disease, Lewy body dementia, frontotemporal dementia, cerebral amyloid angiopathy, primary age-related tauopathy, chronic traumatic encephalopathy, Parkinson's disease, postencephalitic parkinsonism, Huntington's disease, amyolateral sclerosis, Pick's disease, progressive supranuclear palsy, corticobasal degeneration, Lytico-Bodig disease, ganglioglioma and gangliocytoma, subacute sclerosing panencephalitis, Hallervorden-Spatz disease, and/or Creutzfeldt-Jakob disease.

Additionally, these peptides, compositions, and methods may also be used to prevent these neurodegenerative diseases in at-risk populations, such as subjects with Down syndrome and those suffered from brain injuries or cerebral ischemia, as well as the aging population.

### Definitions

As used in the specification and claims, the singular form “a,” “an,” and “the” include plural references unless the context clearly dictates otherwise. For example, the term “a cell” includes a plurality of cells, including mixtures thereof.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

The terms “protein,” “peptide,” and “polypeptide” are used interchangeably to refer to a natural or synthetic molecule comprising two or more amino acids linked by the carboxyl group of one amino acid to the alpha amino group of another. The term “protein” includes amino acids joined to each other by peptide bonds or modified peptide bonds, e.g., peptide isosteres, etc., and can contain modified amino acids other than the 20 gene-encoded amino acids. The polypeptides can be modified by either natural processes, such as post-translational processing, or by chemical modification techniques which are well known in the art. The term also includes peptidomimetics and cyclic peptides.

As used herein, “peptidomimetic” means a mimetic of a peptide which includes some alteration of the normal peptide chemistry. Peptidomimetics typically enhance some property of the original peptide, such as increase stability, increased efficacy, enhanced delivery, increased half life, etc. Methods of making peptidomimetics based upon a known polypeptide sequence is described, for example, in U.S. Pat. Nos. 5,631,280; 5,612,895; and 5,579,250. Use of peptidomimetics can involve the incorporation of a non-amino acid residue with non-amide linkages at a given position. One embodiment of the present invention is a peptidomimetic wherein the compound has a bond, a peptide backbone or an amino acid component replaced with a suitable mimic. Some non-limiting examples of unnatural amino acids which may be suitable amino acid mimics include β-alanine, L-α-amino butyric acid, L-γ-amino butyric acid, L-α-amino isobutyric acid, L-ε-amino caproic acid, 7-amino heptanoic acid, L-aspartic acid, L-glutamic acid, N-ε-Boc-N-α-CBZ-L-lysine, N-ε-Boc-N-α-Fmoc-L-lysine, L-methionine sulfone, L-norleucine, L-norvaline, N-α-Boc-N-δCBZ-L-ornithine, N-δ-Boc-N-α-CBZ-L-ornithine, Boc-p-nitro-L-phenylalanine, Boc-hydroxyproline, and Boc-L-thioproline.

A “fusion protein” refers to a polypeptide formed by the joining of two or more polypeptides through a peptide bond formed between the amino terminus of one polypeptide and the carboxyl terminus of another polypeptide. The fusion protein can be formed by the chemical coupling of the constituent polypeptides or it can be expressed as a single polypeptide from nucleic acid sequence encoding the single contiguous fusion protein. A single chain fusion protein is a fusion protein having a single contiguous polypeptide backbone. Fusion proteins can be prepared using conventional techniques in molecular biology to join the two genes in frame into a single nucleic acid, and then expressing the nucleic acid in an appropriate host cell under conditions in which the fusion protein is produced.

As used herein, protein “binding” is the binding of one protein to another. The binding may comprise covalent bonds, protein cross-linking, and/or non-covalent interactions such as hydrophobic interactions, ionic interactions, or hydrogen bonds.

The term “protein domain” refers to a portion of a protein, portions of a protein, or an entire protein showing structural integrity; this determination may be based on amino acid composition of a portion of a protein, portions of a protein, or the entire protein.

“Amyloid precursor protein” (APP) is an integral membrane protein expressed in many tissues and concentrated in the synapses of neurons. It has been implicated as a regulator of synapse formation, neural plasticity and iron export. APP is cleaved by beta secretase and gamma secretase to yield Aβ. Amyloid beta (Aβ) denotes peptides of 36-43 amino acids that are involved in Alzheimer's disease as the main component of the amyloid plaques found in the brains of Alzheimer patients. Aβ molecules cleaved from APP can aggregate to form flexible soluble oligomers which may exist in various forms. Certain misfolded oligomers (known as “seeds”) can induce other Aβ molecules to also take the misfolded oligomeric form, leading to a chain reaction and buildup of amyloid plaques. The seeds or the resulting amyloid plaques are toxic to cells in the brain.

“Protein tyrosine phosphatases” or “receptor protein tyrosine phosphatases” (PTPs) are a group of enzymes that remove phosphate groups from phosphorylated tyrosine residues on proteins. Protein tyrosine phosphorylation is a common post-translational modification that can create novel recognition motifs for protein interactions and cellular localization, affect protein stability, and regulate enzyme activity. As a consequence, maintaining an appropriate level of protein tyrosine phosphorylation is essential for many cellular functions. Tyrosine-specific protein phosphatases catalyze the removal of a phosphate group attached to a tyrosine residue. These enzymes are key regulatory components in many signal transduction pathways (such as the MAP kinase pathway) that underlie cellular functions such as cell cycle control/proliferation, cell death, differentiation, transformation, cell polarity and motility, synaptic plasticity, etc.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician. An “at-risk” subject is an individual with a higher likelihood of developing a certain disease or condition. An “at-risk” subject may have, for example, received a medical diagnosis associated with the certain disease or condition.

“Tau proteins” (or τ proteins) are proteins that stabilize microtubules. They are abundant in neurons of the central nervous system and are less common elsewhere, but are also expressed at very low levels in CNS astrocytes and oligodendrocytes. Neurodegenerative disorders such as Alzheimer's disease, Parkinson's disease, and other tauopathies are associated with tau proteins that have become defective, misfolded, tangled, and no longer stabilize microtubules properly.

The term “protein fragment” refers to a functional portion of a full-length protein. For example, a fragment of APP or PTPσ may be synthesized chemically or biologically for the purposes of disrupting the binding between APP and PTPσ. Such fragments could be used as “decoy” peptides to prevent or diminish the actual APP-PTPσ binding interaction that results in β-cleavage of APP and subsequent AO formation.

The phrase “functional fragment” or “analog” or mimetic of a protein or other molecule is a compound having qualitative biological activity in common with a full-length protein or other molecule of its entire structure. A functional fragment of a full-length protein may be isolated and attached to a separate peptide sequence. For example, a functional fragment of a blood-brain barrier penetrating protein may be isolated and attached to the decoy peptide that disrupts APP-PTPσ binding, thereby enabling the hybrid peptide to enter the brain and disrupt APP-PTPσ binding. Another example of a functional fragment is a membrane penetrating fragment, or one that relays an ability to pass the lipophilic barrier of a cell's plasma membrane. An analog of heparin, for example, may be a compound that binds to a heparin binding site.

As used herein, “cyclic peptide” or “cyclopeptide” in general refers to a peptide comprising at least one internal bond attaching nonadjacent amino acids of the peptide, such as when the end amino acids of a linear sequence are attached to form a circular peptide.

The term “antibody” refers to natural or synthetic antibodies that selectively bind a target antigen. The term includes polyclonal and monoclonal antibodies. In addition to intact immunoglobulin molecules, also included in the term “antibodies” are fragments or polymers of those immunoglobulin molecules, and human or humanized versions of immunoglobulin molecules that selectively bind the target antigen.

As used herein, “enzyme” refers to a protein specialized to catalyze or promote a specific metabolic reaction.

“Neurodegenerative disorders” or “neurodegenerative diseases” are conditions marked by the progressive loss of structure or function of neural cells, including death of neurons and glia.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “administering” refers to an administration that is intranasal, oral, topical, intravenous, subcutaneous, transcutaneous, transdermal, intramuscular, intra joint, parenteral, intra-arteriole, intradermal, intraventricular, intracranial, intraperitoneal, intralesional, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

The term “pharmaceutically acceptable carrier” means a carrier or excipient that is useful in preparing a pharmaceutical composition that is generally safe and non-toxic, and includes a carrier that is acceptable for veterinary and/or human pharmaceutical use. As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. As used herein, the term “carrier” encompasses any excipient, diluent, filler, salt, buffer, stabilizer, solubilizer, lipid, stabilizer, or other material well known in the art for use in pharmaceutical formulations and as described further below. The pharmaceutical compositions also can include preservatives. A “pharmaceutically acceptable carrier” as used in the specification and claims includes both one and more than one such carrier.

The term “variant” refers to an amino acid or peptide sequence having conservative amino acid substitutions (“conservative variant”), non-conservative amino acid subsitutions (e.g., a degenerate variant), substitutions within the wobble position of each codon (i.e. DNA and RNA) encoding an amino acid, amino acids added to the C-terminus of a peptide, or a peptide having 60%, 70%, 80%, 90%, or 95% homology to a reference sequence.

The term “percent (%) sequence identity” or “homology” is defined as the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

Compositions

### Peptides:

Disclosed herein are peptides for treating and preventing the aforementioned neurodegenerative diseases, such as Alzheimer's disease. In some embodiments, the peptides disrupt the binding between PTPσ and APP, preventing β-amyloidogenic processing of APP without affecting other major substrates of the β- and γ-secretases. The peptide may be a decoy fragment of APP, a decoy fragment of PTPσ, or a combination thereof.

In some embodiments, a decoy peptide could be fabricated from the PTPσ-binding region on APP, which is the fragment between its E1 and E2 domains (SEQ ID NO:1). In some embodiments, a decoy peptide could be fabricated from the APP-binding region on PTPσ, which is its IG1 domain (SEQ ID NO: 442). In some embodiments, a decoy peptide could be fabricated that corresponds to the entire APP E2 domain or a fragment thereof. In some embodiments, a decoy peptide could be fabricated that corresponds to the entire APP E1 domain or a fragment thereof. In some embodiments, a PTPσ peptide is used in combination with an APP peptide.

In some embodiments, the peptide is a fragment of the PTPσ-binding domain of APP. Therefore, in some embodiments, the peptide is a fragment of SEQ ID NO:1, as listed below, which has at least 5, 6, 7, 8, 9, 10, 11, 12, 13, 14, or more amino acids, or a conservative variant thereof.

(SEQ ID NO: 1)
AEESDNVDSADAEEDDSDVWWGGADTDVADGSEDKVVEVAE
EEEVAEVEEEEADDDEDDEDGDEVEEEAEEPYEEATERTTS
IATTTTTTTESVEEVVR.

Therefore, in some embodiments, the peptide comprises an amino acid sequence selected from 10 consecutive residues of SEQ ID NO: 1, or from the group consisting of the below:

SEQ ID NO: 2
AEESDNVDSA
SEQ ID NO: 3
EESDNVDSAD
SEQ ID NO: 4
ESDNVDSADA
SEQ ID NO: 5
SDNVDSADAE
SEQ ID NO: 6
DNVDSADAEE
SEQ ID NO: 7
NVDSADAEED
SEQ ID NO: 8
VDSADAEEDD
SEQ ID NO: 9
DSADAEEDDS
SEQ ID NO: 10
SADAEEDDSD
SEQ ID NO: 11
ADAEEDDSDV
SEQ ID NO: 12
DAEEDDSDVW
SEQ ID NO: 13
AEEDDSDVWW
SEQ ID NO: 14
EEDDSDVWWG
SEQ ID NO: 15
EDDSDVWWGG
SEQ ID NO: 16
DDSDVWWGGA
SEQ ID NO: 17
DSDVWWGGAD
SEQ ID NO: 18
SDVWWGGADT
SEQ ID NO: 19
DVWWGGADTD
SEQ ID NO: 20
VWWGGADTDY
SEQ ID NO: 21
WWGGADTDYA
SEQ ID NO: 22
WGGADTDYAD
SEQ ID NO: 23
GGADTDYADG
SEQ ID NO: 24
GADTDYADGS
SEQ ID NO: 25
ADTDYADGSE
SEQ ID NO: 26
DTDYADGSED
SEQ ID NO: 27
TDYADGSEDK
SEQ ID NO: 28
DYADGSEDKV
SEQ ID NO: 29
YADGSEDKVV
SEQ ID NO: 30
ADGSEDKVVE
SEQ ID NO: 31
DGSEDKVVEV
SEQ ID NO: 32
GSEDKVVEVA
SEQ ID NO: 33
SEDKVVEVAE
SEQ ID NO: 34
EDKVVEVAEE
SEQ ID NO: 35
DKVVEVAEEE
SEQ ID NO: 36
KVVEVAEEEE
SEQ ID NO: 37
VVEVAEEEEV
SEQ ID NO: 38
VEVAEEEEVA
SEQ ID NO: 39
EVAEEEEVAE
SEQ ID NO: 40
VAEEEEVAEV
SEQ ID NO: 41
AEEEEVAEVE
SEQ ID NO: 42
EEEEVAEVEE
SEQ ID NO: 43
EEEVAEVEEE
SEQ ID NO: 44
EEVAEVEEEE
SEQ ID NO: 45
EVAEVEEEEA
SEQ ID NO: 46
VAEVEEEEAD
SEQ ID NO: 47
AEVEEEEADD
SEQ ID NO: 48
EVEEEEADDD
SEQ ID NO: 49
VEEEEADDDE
SEQ ID NO: 50
EEEEADDDED
SEQ ID NO: 51
EEEADDDEDD
SEQ ID NO: 52
EEADDDEDDE
SEQ ID NO: 53
EADDDEDDED
SEQ ID NO: 54
ADDDEDDEDG
SEQ ID NO: 55
DDDEDDEDGD
SEQ ID NO: 56
DDEDDEDGDE
SEQ ID NO: 57
DEDDEDGDEV
SEQ ID NO: 58
EDDEDGDEVE
SEQ ID NO: 59
DDEDGDEVEE
SEQ ID NO: 60
DEDGDEVEEE
SEQ ID NO: 61
EDGDEVEEEA
SEQ ID NO: 62
DGDEVEEEAE
SEQ ID NO: 63
GDEVEEEAEE
SEQ ID NO: 64
DEVEEEAEEP
SEQ ID NO: 65
EVEEEAEEPY
SEQ ID NO: 66
VEEEAEEPYE
SEQ ID NO: 67
EEEAEEPYEE
SEQ ID NO: 68
EEAEEPYEEA
SEQ ID NO: 69
EAEEPYEEAT
SEQ ID NO: 70
AEEPYEEATE
SEQ ID NO: 71
EEPYEEATER
SEQ ID NO: 72
EPYEEATERT
SEQ ID NO: 73
PYEEATERTT
SEQ ID NO: 74
YEEATERTTS
SEQ ID NO: 75
EEATERTTSI
SEQ ID NO: 76
EATERTTSIA
SEQ ID NO: 77
ATERTTSIAT
SEQ ID NO: 78
TERTTSIATT
SEQ ID NO: 79
ERTTSIATTT
SEQ ID NO: 80
RTTSIATTTT
SEQ ID NO: 81
TTSIATTTTT
SEQ ID NO: 82
TSIATTTTTT
SEQ ID NO: 83
SIATTTTTTT
SEQ ID NO: 84
IATTTTTTTE
SEQ ID NO: 85
ATTTTTTTES
SEQ ID NO: 86
TTTTTTTESV
SEQ ID NO: 87
TTTTTTESVE
SEQ ID NO: 88
TTTTTESVEE
SEQ ID NO: 89
TTTTESVEEV
SEQ ID NO: 90
TTTESVEEVV
SEQ ID NO: 91
TTESVEEVVR

In some embodiments, the peptide comprises an amino acid sequence selected from 11 consecutive residues of SEQ ID NO: 1, or from the group consisting of the below:

SEQ ID NO: 92
AEESDNVDSAD
SEQ ID NO: 93
EESDNVDSADA
SEQ ID NO: 94
ESDNVDSADAE
SEQ ID NO: 95
SDNVDSADAEE
SEQ ID NO: 96
DNVDSADAEED
SEQ ID NO: 97
NVDSADAEEDD
SEQ ID NO: 98
VDSADAEEDDS
SEQ ID NO: 99
DSADAEEDDSD
SEQ ID NO: 100
SADAEEDDSDV
SEQ ID NO: 101
ADAEEDDSDVW
SEQ ID NO: 102
DAEEDDSDVWW
SEQ ID NO: 103
AEEDDSDVWWG
SEQ ID NO: 104
EEDDSDVWWGG
SEQ ID NO: 105
EDDSDVWWGGA
SEQ ID NO: 106
DDSDVWWGGAD
SEQ ID NO: 107
DSDVWWGGADT
SEQ ID NO: 108
SDVWWGGADTD
SEQ ID NO: 109
DVWWGGADTDY
SEQ ID NO: 110
VWWGGADTDYA
SEQ ID NO: 111
WWGGADTDYAD
SEQ ID NO: 112
WGGADTDYADG
SEQ ID NO: 113
GGADTDYADGS
SEQ ID NO: 114
GADTDYADGSE
SEQ ID NO: 115
ADTDYADGSED
SEQ ID NO: 116
DTDYADGSEDK
SEQ ID NO: 117
TDYADGSEDKV
SEQ ID NO: 118
DYADGSEDKVV
SEQ ID NO: 119
YADGSEDKVVE
SEQ ID NO: 120
ADGSEDKVVEV
SEQ ID NO: 121
DGSEDKVVEVA
SEQ ID NO: 122
GSEDKVVEVAE
SEQ ID NO: 123
SEDKVVEVAEE
SEQ ID NO: 124
EDKVVEVAEEE
SEQ ID NO: 125
DKVVEVAEEEE
SEQ ID NO: 126
KVVEVAEEEEV
SEQ ID NO: 127
VVEVAEEEEVA
SEQ ID NO: 128
VEVAEEEEVAE
SEQ ID NO: 129
EVAEEEEVAEV
SEQ ID NO: 130
VAEEEEVAEVE
SEQ ID NO: 131
AEEEEVAEVEE
SEQ ID NO: 132
EEEEVAEVEEE
SEQ ID NO: 133
EEEVAEVEEEE
SEQ ID NO: 134
EEVAEVEEEEA
SEQ ID NO: 135
EVAEVEEEEAD
SEQ ID NO: 136
VAEVEEEEADD
SEQ ID NO: 137
AEVEEEEADDD
SEQ ID NO: 138
EVEEEEADDDE
SEQ ID NO: 139
VEEEEADDDED
SEQ ID NO: 140
EEEEADDDEDD
SEQ ID NO: 141
EEEADDDEDDE
SEQ ID NO: 142
EEADDDEDDED
SEQ ID NO: 143
EADDDEDDEDG
SEQ ID NO: 144
ADDDEDDEDGD
SEQ ID NO: 145
DDDEDDEDGDE
SEQ ID NO: 146
DDEDDEDGDEV
SEQ ID NO: 147
DEDDEDGDEVE
SEQ ID NO: 148
EDDEDGDEVEE
SEQ ID NO: 149
DDEDGDEVEEE
SEQ ID NO: 150
DEDGDEVEEEA
SEQ ID NO: 151
EDGDEVEEEAE
SEQ ID NO: 152
DGDEVEEEAEE
SEQ ID NO: 153
GDEVEEEAEEP
SEQ ID NO: 154
DEVEEEAEEPY
SEQ ID NO: 155
EVEEEAEEPYE
SEQ ID NO: 156
VEEEAEEPYEE
SEQ ID NO: 157
EEEAEEPYEEA
SEQ ID NO: 158
EEAEEPYEEAT
SEQ ID NO: 159
EAEEPYEEATE
SEQ ID NO: 160
AEEPYEEATER
SEQ ID NO: 161
EEPYEEATERT
SEQ ID NO: 162
EPYEEATERTT
SEQ ID NO: 163
PYEEATERTTS
SEQ ID NO: 164
YEEATERTTSI
SEQ ID NO: 165
EEATERTTSIA
SEQ ID NO: 166
EATERTTSIAT
SEQ ID NO: 167
ATERTTSIATT
SEQ ID NO: 168
TERTTSIATTT
SEQ ID NO: 169
ERTTSIATTTT
SEQ ID NO: 170
RTTSIATTTTT
SEQ ID NO: 171
TTSIATTTTTT
SEQ ID NO: 172
TSIATTTTTTT
SEQ ID NO: 173
SIATTTTTTTE
SEQ ID NO: 174
IATTTTTTTES
SEQ ID NO: 175
ATTTTTTTESV
SEQ ID NO: 176
TTTTTTTESVE
SEQ ID NO: 177
TTTTTTESVEE
SEQ ID NO: 178
TTTTTESVEEV
SEQ ID NO: 179
TTTTESVEEVV
SEQ ID NO: 180
TTTESVEEVVR

In some embodiments, the peptide comprises an amino acid sequence selected from 12 consecutive residues of SEQ ID NO: 1, or from the group consisting of the below:

SEQ ID NO: 181
AEESDNVDSADA
SEQ ID NO: 182
EESDNVDSADAE
SEQ ID NO: 183
ESDNVDSADAEE
SEQ ID NO: 184
SDNVDSADAEED
SEQ ID NO: 185
DNVDSADAEEDD
SEQ ID NO: 186
NVDSADAEEDDS
SEQ ID NO: 187
VDSADAEEDDSD
SEQ ID NO: 188
DSADAEEDDSDV
SEQ ID NO: 189
SADAEEDDSDVW
SEQ ID NO: 190
ADAEEDDSDVWW
SEQ ID NO: 191
DAEEDDSDVWWG
SEQ ID NO: 192
AEEDDSDVWWGG
SEQ ID NO: 193
EEDDSDVWWGGA
SEQ ID NO: 194
EDDSDVWWGGAD
SEQ ID NO: 195
DDSDVWWGGADT
SEQ ID NO: 196
DSDVWWGGADTD
SEQ ID NO: 197
SDVWWGGADTDY
SEQ ID NO: 198
DVWWGGADTDYA
SEQ ID NO: 199
VWWGGADTDYAD
SEQ ID NO: 200
WWGGADTDYADG
SEQ ID NO: 201
WGGADTDYADGS
SEQ ID NO: 202
GGADTDYADGSE
SEQ ID NO: 203
GADTDYADGSED
SEQ ID NO: 204
ADTDYADGSEDK
SEQ ID NO: 205
DTDYADGSEDKV
SEQ ID NO: 206
TDYADGSEDKVV
SEQ ID NO: 207
DYADGSEDKVVE
SEQ ID NO: 208
YADGSEDKVVEV
SEQ ID NO: 209
ADGSEDKVVEVA
SEQ ID NO: 210
DGSEDKVVEVAE
SEQ ID NO: 211
GSEDKVVEVAEE
SEQ ID NO: 212
SEDKVVEVAEEE
SEQ ID NO: 213
EDKVVEVAEEEE
SEQ ID NO: 214
DKVVEVAEEEEV
SEQ ID NO: 215
KVVEVAEEEEVA
SEQ ID NO: 216
VVEVAEEEEVAE
SEQ ID NO: 217
VEVAEEEEVAEV
SEQ ID NO: 218
EVAEEEEVAEVE
SEQ ID NO: 219
VAEEEEVAEVEE
SEQ ID NO: 220
AEEEEVAEVEEE
SEQ ID NO: 221
EEEEVAEVEEEE
SEQ ID NO: 222
EEEVAEVEEEEA
SEQ ID NO: 223
EEVAEVEEEEAD
SEQ ID NO: 224
EVAEVEEEEADD
SEQ ID NO: 225
VAEVEEEEADDD
SEQ ID NO: 226
AEVEEEEADDDE
SEQ ID NO: 227
EVEEEEADDDED
SEQ ID NO: 228
VEEEEADDDEDD
SEQ ID NO: 229
EEEEADDDEDDE
SEQ ID NO: 230
EEEADDDEDDED
SEQ ID NO: 231
EEADDDEDDEDG
SEQ ID NO: 232
EADDDEDDEDGD
SEQ ID NO: 233
ADDDEDDEDGDE
SEQ ID NO: 234
DDDEDDEDGDEV
SEQ ID NO: 235
DDEDDEDGDEVE
SEQ ID NO: 236
DEDDEDGDEVEE
SEQ ID NO: 237
EDDEDGDEVEEE
SEQ ID NO: 238
DDEDGDEVEEEA
SEQ ID NO: 239
DEDGDEVEEEAE
SEQ ID NO: 240
EDGDEVEEEAEE
SEQ ID NO: 241
DGDEVEEEAEEP
SEQ ID NO: 242
GDEVEEEAEEPY
SEQ ID NO: 243
DEVEEEAEEPYE
SEQ ID NO: 244
EVEEEAEEPYEE
SEQ ID NO: 245
VEEEAEEPYEEA
SEQ ID NO: 246
EEEAEEPYEEAT
SEQ ID NO: 247
EEAEEPYEEATE
SEQ ID NO: 248
EAEEPYEEATER
SEQ ID NO: 249
AEEPYEEATERT
SEQ ID NO: 250
EEPYEEATERTT
SEQ ID NO: 251
EPYEEATERTTS
SEQ ID NO: 252
PYEEATERTTSI
SEQ ID NO: 253
YEEATERTTSIA
SEQ ID NO: 254
EEATERTTSIAT
SEQ ID NO: 255
EATERTTSIATT
SEQ ID NO: 256
ATERTTSIATTT
SEQ ID NO: 257
TERTTSIATTTT
SEQ ID NO: 258
ERTTSIATTTTT
SEQ ID NO: 259
RTTSIATTTTTT
SEQ ID NO: 260
TTSIATTTTTTT
SEQ ID NO: 261
TSIATTTTTTTE
SEQ ID NO: 262
SIATTTTTTTES
SEQ ID NO: 263
IATTTTTTTESV
SEQ ID NO: 264
ATTTTTTTESVE
SEQ ID NO: 265
TTTTTTTESVEE
SEQ ID NO: 266
TTTTTTESVEEV
SEQ ID NO: 267
TTTTTESVEEVV
SEQ ID NO: 268
TTTTESVEEVVR

In some embodiments, the peptide comprises an amino acid sequence selected from 13 consecutive residues of SEQ ID NO: 1, or from the group consisting of the below:

SEQ ID NO: 268
TTTTESVEEVVR
SEQ ID NO: 269
AEESDNVDSADAE
SEQ ID NO: 270
EESDNVDSADAEE
SEQ ID NO: 271
ESDNVDSADAEED
SEQ ID NO: 272
SDNVDSADAEEDD
SEQ ID NO: 273
DNVDSADAEEDDS
SEQ ID NO: 274
NVDSADAEEDDSD
SEQ ID NO: 275
VDSADAEEDDSDV
SEQ ID NO: 276
DSADAEEDDSDVW
SEQ ID NO: 277
SADAEEDDSDVWW
SEQ ID NO: 278
ADAEEDDSDVWWG
SEQ ID NO: 279
DAEEDDSDVWWGG
SEQ ID NO: 280
AEEDDSDVWWGGA
SEQ ID NO: 281
EEDDSDVWWGGAD
SEQ ID NO: 282
EDDSDVWWGGADT
SEQ ID NO: 283
DDSDVWWGGADTD
SEQ ID NO: 284
DSDVWWGGADTDY
SEQ ID NO: 285
SDVWWGGADTDYA
SEQ ID NO: 286
DVWWGGADTDYAD
SEQ ID NO: 287
VWWGGADTDYADG
SEQ ID NO: 288
WWGGADTDYADGS
SEQ ID NO: 289
WGGADTDYADGSE
SEQ ID NO: 290
GGADTDYADGSED
SEQ ID NO: 291
GADTDYADGSEDK
SEQ ID NO: 292
ADTDYADGSEDKV
SEQ ID NO: 293
DTDYADGSEDKVV
SEQ ID NO: 294
TDYADGSEDKVVE
SEQ ID NO: 295
DYADGSEDKVVEV
SEQ ID NO: 296
YADGSEDKVVEVA
SEQ ID NO: 297
ADGSEDKVVEVAE
SEQ ID NO: 298
DGSEDKVVEVAEE
SEQ ID NO: 299
GSEDKVVEVAEEE
SEQ ID NO: 300
SEDKVVEVAEEEE
SEQ ID NO: 301
EDKVVEVAEEEEV
SEQ ID NO: 302
DKVVEVAEEEEVA
SEQ ID NO: 303
KVVEVAEEEEVAE
SEQ ID NO: 304
VVEVAEEEEVAEV
SEQ ID NO: 305
VEVAEEEEVAEVE
SEQ ID NO: 306
EVAEEEEVAEVEE
SEQ ID NO: 307
VAEEEEVAEVEEE
SEQ ID NO: 308
AEEEEVAEVEEEE
SEQ ID NO: 309
EEEEVAEVEEEEA
SEQ ID NO: 310
EEEVAEVEEEEAD
SEQ ID NO: 311
EEVAEVEEEEADD
SEQ ID NO: 312
EVAEVEEEEADDD
SEQ ID NO: 313
VAEVEEEEADDDE
SEQ ID NO: 314
AEVEEEEADDDED
SEQ ID NO: 315
EVEEEEADDDEDD
SEQ ID NO: 316
VEEEEADDDEDDE
SEQ ID NO: 317
EEEEADDDEDDED
SEQ ID NO: 318
EEEADDDEDDEDG
SEQ ID NO: 319
EEADDDEDDEDGD
SEQ ID NO: 320
EADDDEDDEDGDE
SEQ ID NO: 321
ADDDEDDEDGDEV
SEQ ID NO: 322
DDDEDDEDGDEVE
SEQ ID NO: 323
DDEDDEDGDEVEE
SEQ ID NO: 324
DEDDEDGDEVEEE
SEQ ID NO: 325
EDDEDGDEVEEEA
SEQ ID NO: 326
DDEDGDEVEEEAE
SEQ ID NO: 327
DEDGDEVEEEAEE
SEQ ID NO: 328
EDGDEVEEEAEEP
SEQ ID NO: 329
DGDEVEEEAEEPY
SEQ ID NO: 330
GDEVEEEAEEPYE
SEQ ID NO: 331
DEVEEEAEEPYEE
SEQ ID NO: 332
EVEEEAEEPYEEA
SEQ ID NO: 333
VEEEAEEPYEEAT
SEQ ID NO: 334
EEEAEEPYEEATE
SEQ ID NO: 335
EEAEEPYEEATER
SEQ ID NO: 336
EAEEPYEEATERT
SEQ ID NO: 337
AEEPYEEATERTT
SEQ ID NO: 338
EEPYEEATERTTS
SEQ ID NO: 339
EPYEEATERTTSI
SEQ ID NO: 340
PYEEATERTTSIA
SEQ ID NO: 341
YEEATERTTSIAT
SEQ ID NO: 342
EEATERTTSIATT
SEQ ID NO: 343
EATERTTSIATTT
SEQ ID NO: 344
ATERTTSIATTTT
SEQ ID NO: 345
TERTTSIATTTTT
SEQ ID NO: 346
ERTTSIATTTTTT
SEQ ID NO: 347
RTTSIATTTTTTT
SEQ ID NO: 348
TTSIATTTTTTTE
SEQ ID NO: 349
TSIATTTTTTTES
SEQ ID NO: 350
SIATTTTTTTESV
SEQ ID NO: 351
IATTTTTTTESVE
SEQ ID NO: 352
ATTTTTTTESVEE
SEQ ID NO: 353
TTTTTTTESVEEV
SEQ ID NO: 354
TTTTTTESVEEVV
SEQ ID NO: 355
TTTTTESVEEVVR

In some embodiments, the peptide comprises an amino acid sequence selected from 14 consecutive residues of SEQ ID NO: 1, or from the group consisting of the below:

SEQ ID NO: 356
AEESDNVDSADAEE
SEQ ID NO: 357
EESDNVDSADAEED
SEQ ID NO: 358
ESDNVDSADAEEDD
SEQ ID NO: 359
SDNVDSADAEEDDS
SEQ ID NO: 360
DNVDSADAEEDDSD
SEQ ID NO: 361
NVDSADAEEDDSDV
SEQ ID NO: 362
VDSADAEEDDSDVW
SEQ ID NO: 363
DSADAEEDDSDVWW
SEQ ID NO: 364
SADAEEDDSDVWWG
SEQ ID NO: 365
ADAEEDDSDVWWGG
SEQ ID NO: 366
DAEEDDSDVWWGGA
SEQ ID NO: 367
AEEDDSDVWWGGAD
SEQ ID NO: 368
EEDDSDVWWGGADT
SEQ ID NO: 369
EDDSDVWWGGADTD
SEQ ID NO: 370
DDSDVWWGGADTDY
SEQ ID NO: 371
DSDVWWGGADTDYA
SEQ ID NO: 372
SDVWWGGADTDYAD
SEQ ID NO: 373
DVWWGGADTDYADG
SEQ ID NO: 374
VWWGGADTDYADGS
SEQ ID NO: 375
WWGGADTDYADGSE
SEQ ID NO: 376
WGGADTDYADGSED
SEQ ID NO: 377
GGADTDYADGSEDK
SEQ ID NO: 378
GADTDYADGSEDKV
SEQ ID NO: 379
ADTDYADGSEDKVV
SEQ ID NO: 380
DTDYADGSEDKVVE
SEQ ID NO: 381
TDYADGSEDKVVEV
SEQ ID NO: 382
DYADGSEDKVVEVA
SEQ ID NO: 383
YADGSEDKVVEVAE
SEQ ID NO: 384
ADGSEDKVVEVAEE
SEQ ID NO: 385
DGSEDKVVEVAEEE
SEQ ID NO: 386
GSEDKVVEVAEEEE
SEQ ID NO: 387
SEDKVVEVAEEEEV
SEQ ID NO: 388
EDKVVEVAEEEEVA
SEQ ID NO: 389
DKVVEVAEEEEVAE
SEQ ID NO: 390
KVVEVAEEEEVAEV
SEQ ID NO: 391
VVEVAEEEEVAEVE
SEQ ID NO: 392
VEVAEEEEVAEVEE
SEQ ID NO: 393
EVAEEEEVAEVEEE
SEQ ID NO: 394
VAEEEEVAEVEEEE
SEQ ID NO: 395
AEEEEVAEVEEEEA
SEQ ID NO: 396
EEEEVAEVEEEEAD
SEQ ID NO: 397
EEEVAEVEEEEADD
SEQ ID NO: 398
EEVAEVEEEEADDD
SEQ ID NO: 399
EVAEVEEEEADDDE
SEQ ID NO: 400
VAEVEEEEADDDED
SEQ ID NO: 401
AEVEEEEADDDEDD
SEQ ID NO: 402
EVEEEEADDDEDDE
SEQ ID NO: 403
VEEEEADDDEDDED
SEQ ID NO: 404
EEEEADDDEDDEDG
SEQ ID NO: 405
EEEADDDEDDEDGD
SEQ ID NO: 406
EEADDDEDDEDGDE
SEQ ID NO: 407
EADDDEDDEDGDEV
SEQ ID NO: 408
ADDDEDDEDGDEVE
SEQ ID NO: 409
DDDEDDEDGDEVEE
SEQ ID NO: 410
DDEDDEDGDEVEEE
SEQ ID NO: 411
DEDDEDGDEVEEEA
SEQ ID NO: 412
EDDEDGDEVEEEAE
SEQ ID NO: 413
DDEDGDEVEEEAEE
SEQ ID NO: 414
DEDGDEVEEEAEEP
SEQ ID NO: 415
EDGDEVEEEAEEPY
SEQ ID NO: 416
DGDEVEEEAEEPYE
SEQ ID NO: 417
GDEVEEEAEEPYEE
SEQ ID NO: 418
DEVEEEAEEPYEEA
SEQ ID NO: 419
EVEEEAEEPYEEAT
SEQ ID NO: 420
VEEEAEEPYEEATE
SEQ ID NO: 421
EEEAEEPYEEATER
SEQ ID NO: 422
EEAEEPYEEATERT
SEQ ID NO: 423
EAEEPYEEATERTT
SEQ ID NO: 424
AEEPYEEATERTTS
SEQ ID NO: 425
EEPYEEATERTTSI
SEQ ID NO: 426
EPYEEATERTTSIA
SEQ ID NO: 427
PYEEATERTTSIAT
SEQ ID NO: 428
YEEATERTTSIATT
SEQ ID NO: 429
EEATERTTSIATTT
SEQ ID NO: 430
EATERTTSIATTTT
SEQ ID NO: 431
ATERTTSIATTTTT
SEQ ID NO: 432
TERTTSIATTTTTT
SEQ ID NO: 433
ERTTSIATTTTTTT
SEQ ID NO: 434
RTTSIATTTTTTTE
SEQ ID NO: 435
TTSIATTTTTTTES
SEQ ID NO: 436
TSIATTTTTTTESV
SEQ ID NO: 437
SIATTTTTTTESVE
SEQ ID NO: 438
IATTTTTTTESVEE
SEQ ID NO: 439
ATTTTTTTESVEEV
SEQ ID NO: 440
TTTTTTTESVEEVV
SEQ ID NO: 441
TTTTTTESVEEVVR

In some embodiments, the peptide comprises an amino acid sequence selected from 24 consecutive residues of SEQ ID NO: 1, or from the group consisting of the below:

SEQ ID NO: 900
ATERTTSIATTTTTTTESVEEVVR

In some embodiments, the peptide is a fragment of the APP-binding domain of PTPσ. Therefore, in some embodiments, the peptide is a fragment of SEQ ID NO:442, as listed below, which has at least 5, 6, 7, 8, 9, 10, 11, 12, 13, 14, or more amino acids, or a conservative variant thereof. The underlined amino acids represent residues in the ligand-binding pocket.

(SEQ ID NO: 442)
EEPPRFIKEPKDQIGVSGGVASFVCQATGDPKPRVTWNKKGKKVNSQRFET
IEFDESAGAVLRIQPLRTPRDENVYECVAQNSVGEITVHAKLTVLRE.

Therefore, in some embodiments, the peptide comprises an amino acid sequence selected from 10 consecutive residues of SEQ ID NO: 442, or from the group consisting of the below:

SEQ ID NO: 443
EEPPRFIKEP
SEQ ID NO: 444
EPPRFIKEPK
SEQ ID NO: 445
PPRFIKEPKD
SEQ ID NO: 446
PRFIKEPKDQ
SEQ ID NO: 447
RFIKEPKDQI
SEQ ID NO: 448
FIKEPKDQIG
SEQ ID NO: 449
IKEPKDQIGV
SEQ ID NO: 450
KEPKDQIGVS
SEQ ID NO: 451
EPKDQIGVSG
SEQ ID NO: 452
PKDQIGVSGG
SEQ ID NO: 453
KDQIGVSGGV
SEQ ID NO: 454
DQIGVSGGVA
SEQ ID NO: 455
QIGVSGGVAS
SEQ ID NO: 456
IGVSGGVASF
SEQ ID NO: 457
GVSGGVASFV
SEQ ID NO: 458
VSGGVASFVC
SEQ ID NO: 459
SGGVASFVCQ
SEQ ID NO: 460
GGVASFVCQA
SEQ ID NO: 461
GVASFVCQAT
SEQ ID NO: 462
VASFVCQATG
SEQ ID NO: 463
ASFVCQATGD
SEQ ID NO: 464
SFVCQATGDP
SEQ ID NO: 465
FVCQATGDPK
SEQ ID NO: 466
VCQATGDPKP
SEQ ID NO: 467
CQATGDPKPR
SEQ ID NO: 468
QATGDPKPRV
SEQ ID NO: 469
ATGDPKPRVT
SEQ ID NO: 470
TGDPKPRVTW
SEQ ID NO: 471
GDPKPRVTWN
SEQ ID NO: 472
DPKPRVTWNK
SEQ ID NO: 473
PKPRVTWNKK
SEQ ID NO: 474
KPRVTWNKKG
SEQ ID NO: 475
PRVTWNKKGK
SEQ ID NO: 476
RVTWNKKGKK
SEQ ID NO: 477
VTWNKKGKKV
SEQ ID NO: 478
TWNKKGKKVN
SEQ ID NO: 479
WNKKGKKVNS
SEQ ID NO: 480
NKKGKKVNSQ
SEQ ID NO: 481
KKGKKVNSQR
SEQ ID NO: 482
KGKKVNSQRF
SEQ ID NO: 483
GKKVNSQRFE
SEQ ID NO: 484
KKVNSQRFET
SEQ ID NO: 485
KVNSQRFETI
SEQ ID NO: 486
VNSQRFETIE
SEQ ID NO: 487
NSQRFETIEF
SEQ ID NO: 488
SQRFETIEFD
SEQ ID NO: 489
QRFETIEFDE
SEQ ID NO: 490
RFETIEFDES
SEQ ID NO: 491
FETIEFDESA
SEQ ID NO: 492
ETIEFDESAG
SEQ ID NO: 493
TIEFDESAGA
SEQ ID NO: 494
IEFDESAGAV
SEQ ID NO: 495
EFDESAGAVL
SEQ ID NO: 496
FDESAGAVLR
SEQ ID NO: 497
DESAGAVLRI
SEQ ID NO: 498
ESAGAVLRIQ
SEQ ID NO: 499
SAGAVLRIQP
SEQ ID NO: 500
AGAVLRIQPL
SEQ ID NO: 501
GAVLRIQPLR
SEQ ID NO: 502
AVLRIQPLRT
SEQ ID NO: 503
VLRIQPLRTP
SEQ ID NO: 504
LRIQPLRTPR
SEQ ID NO: 505
RIQPLRTPRD
SEQ ID NO: 506
IQPLRTPRDE
SEQ ID NO: 507
QPLRTPRDEN
SEQ ID NO: 508
PLRTPRDENV
SEQ ID NO: 509
LRTPRDENVY
SEQ ID NO: 510
RTPRDENVYE
SEQ ID NO: 511
TPRDENVYEC
SEQ ID NO: 512
PRDENVYECV
SEQ ID NO: 513
RDENVYECVA
SEQ ID NO: 514
DENVYECVAQ
SEQ ID NO: 515
ENVYECVAQN
SEQ ID NO: 516
NVYECVAQNS
SEQ ID NO: 517
VYECVAQNSV
SEQ ID NO: 518
YECVAQNSVG
SEQ ID NO: 519
ECVAQNSVGE
SEQ ID NO: 520
CVAQNSVGEI
SEQ ID NO: 521
VAQNSVGEIT
SEQ ID NO: 522
AQNSVGEITV
SEQ ID NO: 523
QNSVGEITVH
SEQ ID NO: 524
NSVGEITVHA
SEQ ID NO: 525
SVGEITVHAK
SEQ ID NO: 526
VGEITVHAKL
SEQ ID NO: 527
GEITVHAKLT
SEQ ID NO: 528
EITVHAKLTV
SEQ ID NO: 529
ITVHAKLTVL
SEQ ID NO: 530
TVHAKLTVLR
SEQ ID NO: 531
VHAKLTVLRE

In some embodiments, the peptide comprises an amino acid sequence selected from 11 consecutive residues of SEQ ID NO: 442, or from the group consisting of the below:

SEQ ID NO: 531
VHAKLTVLRE
SEQ ID NO: 532
EEPPRFIKEPK
SEQ ID NO: 533
EPPRFIKEPKD
SEQ ID NO: 534
PPRFIKEPKDQ
SEQ ID NO: 535
PRFIKEPKDQI
SEQ ID NO: 536
RFIKEPKDQIG
SEQ ID NO: 537
FIKEPKDQIGV
SEQ ID NO: 538
IKEPKDQIGVS
SEQ ID NO: 539
KEPKDQIGVSG
SEQ ID NO: 540
EPKDQIGVSGG
SEQ ID NO: 541
PKDQIGVSGGV
SEQ ID NO: 542
KDQIGVSGGVA
SEQ ID NO: 543
DQIGVSGGVAS
SEQ ID NO: 544
QIGVSGGVASF
SEQ ID NO: 545
IGVSGGVASFV
SEQ ID NO: 546
GVSGGVASFVC
SEQ ID NO: 547
VSGGVASFVCQ
SEQ ID NO: 548
SGGVASFVCQA
SEQ ID NO: 549
GGVASFVCQAT
SEQ ID NO: 550
GVASFVCQATG
SEQ ID NO: 551
VASFVCQATGD
SEQ ID NO: 552
ASFVCQATGDP
SEQ ID NO: 553
SFVCQATGDPK
SEQ ID NO: 554
FVCQATGDPKP
SEQ ID NO: 555
VCQATGDPKPR
SEQ ID NO: 556
CQATGDPKPRV
SEQ ID NO: 557
QATGDPKPRVT
SEQ ID NO: 558
ATGDPKPRVTW
SEQ ID NO: 559
TGDPKPRVTWN
SEQ ID NO: 560
GDPKPRVTWNK
SEQ ID NO: 561
DPKPRVTWNKK
SEQ ID NO: 562
PKPRVTWNKKG
SEQ ID NO: 563
KPRVTWNKKGK
SEQ ID NO: 564
PRVTWNKKGKK
SEQ ID NO: 565
RVTWNKKGKKV
SEQ ID NO: 566
VTWNKKGKKVN
SEQ ID NO: 567
TWNKKGKKVNS
SEQ ID NO: 568
WNKKGKKVNSQ
SEQ ID NO: 569
NKKGKKVNSQR
SEQ ID NO: 570
KKGKKVNSQRF
SEQ ID NO: 571
KGKKVNSQRFE
SEQ ID NO: 572
GKKVNSQRFET
SEQ ID NO: 573
KKVNSQRFETI
SEQ ID NO: 574
KVNSQRFETIE
SEQ ID NO: 575
VNSQRFETIEF
SEQ ID NO: 576
NSQRFETIEFD
SEQ ID NO: 577
SQRFETIEFDE
SEQ ID NO: 578
QRFETIEFDES
SEQ ID NO: 579
RFETIEFDESA
SEQ ID NO: 580
FETIEFDESAG
SEQ ID NO: 581
ETIEFDESAGA
SEQ ID NO: 582
TIEFDESAGAV
SEQ ID NO: 583
IEFDESAGAVL
SEQ ID NO: 584
EFDESAGAVLR
SEQ ID NO: 585
FDESAGAVLRI
SEQ ID NO: 586
DESAGAVLRIQ
SEQ ID NO: 587
ESAGAVLRIQP
SEQ ID NO: 588
SAGAVLRIQPL
SEQ ID NO: 589
AGAVLRIQPLR
SEQ ID NO: 590
GAVLRIQPLRT
SEQ ID NO: 591
AVLRIQPLRTP
SEQ ID NO: 592
VLRIQPLRTPR
SEQ ID NO: 593
LRIQPLRTPRD
SEQ ID NO: 594
RIQPLRTPRDE
SEQ ID NO: 595
IQPLRTPRDEN
SEQ ID NO: 596
QPLRTPRDENV
SEQ ID NO: 597
PLRTPRDENVY
SEQ ID NO: 598
LRTPRDENVYE
SEQ ID NO: 599
RTPRDENVYEC
SEQ ID NO: 600
TPRDENVYECV
SEQ ID NO: 601
PRDENVYECVA
SEQ ID NO: 602
RDENVYECVAQ
SEQ ID NO: 603
DENVYECVAQN
SEQ ID NO: 604
ENVYECVAQNS
SEQ ID NO: 605
NVYECVAQNSV
SEQ ID NO: 606
VYECVAQNSVG
SEQ ID NO: 607
YECVAQNSVGE
SEQ ID NO: 608
ECVAQNSVGEI
SEQ ID NO: 609
CVAQNSVGEIT
SEQ ID NO: 610
VAQNSVGEITV
SEQ ID NO: 611
AQNSVGEITVH
SEQ ID NO: 612
QNSVGEITVHA
SEQ ID NO: 613
NSVGEITVHAK
SEQ ID NO: 614
SVGEITVHAKL
SEQ ID NO: 615
VGEITVHAKLT
SEQ ID NO: 616
GEITVHAKLTV
SEQ ID NO: 617
EITVHAKLTVL
SEQ ID NO: 618
ITVHAKLTVLR
SEQ ID NO: 619
TVHAKLTVLRE

In some embodiments, the peptide comprises an amino acid sequence selected from 12 consecutive residues of SEQ ID NO: 442, or from the group consisting of the below:

SEQ ID NO: 620
EEPPRFIKEPKD
SEQ ID NO: 621
EPPRFIKEPKDQ
SEQ ID NO: 622
PPRFIKEPKDQI
SEQ ID NO: 623
PRFIKEPKDQIG
SEQ ID NO: 624
RFIKEPKDQIGV
SEQ ID NO: 625
FIKEPKDQIGVS
SEQ ID NO: 626
IKEPKDQIGVSG
SEQ ID NO: 627
KEPKDQIGVSGG
SEQ ID NO: 628
EPKDQIGVSGGV
SEQ ID NO: 629
PKDQIGVSGGVA
SEQ ID NO: 630
KDQIGVSGGVAS
SEQ ID NO: 631
DQIGVSGGVASF
SEQ ID NO: 632
QIGVSGGVASFV
SEQ ID NO: 633
IGVSGGVASFVC
SEQ ID NO: 634
GVSGGVASFVCQ
SEQ ID NO: 635
VSGGVASFVCQA
SEQ ID NO: 636
SGGVASFVCQAT
SEQ ID NO: 637
GGVASFVCQATG
SEQ ID NO: 638
GVASFVCQATGD
SEQ ID NO: 639
VASFVCQATGDP
SEQ ID NO: 640
ASFVCQATGDPK
SEQ ID NO: 641
SFVCQATGDPKP
SEQ ID NO: 642
FVCQATGDPKPR
SEQ ID NO: 643
VCQATGDPKPRV
SEQ ID NO: 644
CQATGDPKPRVT
SEQ ID NO: 645
QATGDPKPRVTW
SEQ ID NO: 646
ATGDPKPRVTWN
SEQ ID NO: 647
TGDPKPRVTWNK
SEQ ID NO: 648
GDPKPRVTWNKK
SEQ ID NO: 649
DPKPRVTWNKKG
SEQ ID NO: 650
PKPRVTWNKKGK
SEQ ID NO: 651
KPRVTWNKKGKK
SEQ ID NO: 652
PRVTWNKKGKKV
SEQ ID NO: 653
RVTWNKKGKKVN
SEQ ID NO: 654
VTWNKKGKKVNS
SEQ ID NO: 655
TWNKKGKKVNSQ
SEQ ID NO: 656
WNKKGKKVNSQR
SEQ ID NO: 657
NKKGKKVNSQRF
SEQ ID NO: 658
KKGKKVNSQRFE
SEQ ID NO: 659
KGKKVNSQRFET
SEQ ID NO: 660
GKKVNSQRFETI
SEQ ID NO: 661
KKVNSQRFETIE
SEQ ID NO: 662
KVNSQRFETIEF
SEQ ID NO: 663
VNSQRFETIEFD
SEQ ID NO: 664
NSQRFETIEFDE
SEQ ID NO: 665
SQRFETIEFDES
SEQ ID NO: 666
QRFETIEFDESA
SEQ ID NO: 667
RFETIEFDESAG
SEQ ID NO: 668
FETIEFDESAGA
SEQ ID NO: 669
ETIEFDESAGAV
SEQ ID NO: 670
TIEFDESAGAVL
SEQ ID NO: 671
IEFDESAGAVLR
SEQ ID NO: 672
EFDESAGAVLRI
SEQ ID NO: 673
FDESAGAVLRIQ
SEQ ID NO: 674
DESAGAVLRIQP
SEQ ID NO: 675
ESAGAVLRIQPL
SEQ ID NO: 676
SAGAVLRIQPLR
SEQ ID NO: 677
AGAVLRIQPLRT
SEQ ID NO: 678
GAVLRIQPLRTP
SEQ ID NO: 679
AVLRIQPLRTPR
SEQ ID NO: 680
VLRIQPLRTPRD
SEQ ID NO: 681
LRIQPLRTPRDE
SEQ ID NO: 682
RIQPLRTPRDEN
SEQ ID NO: 683
IQPLRTPRDENV
SEQ ID NO: 684
QPLRTPRDENVY
SEQ ID NO: 685
PLRTPRDENVYE
SEQ ID NO: 686
LRTPRDENVYEC
SEQ ID NO: 687
RTPRDENVYECV
SEQ ID NO: 688
TPRDENVYECVA
SEQ ID NO: 689
PRDENVYECVAQ
SEQ ID NO: 690
RDENVYECVAQN
SEQ ID NO: 691
DENVYECVAQNS
SEQ ID NO: 692
ENVYECVAQNSV
SEQ ID NO: 693
NVYECVAQNSVG
SEQ ID NO: 694
VYECVAQNSVGE
SEQ ID NO: 695
YECVAQNSVGEI
SEQ ID NO: 696
ECVAQNSVGEIT
SEQ ID NO: 697
CVAQNSVGEITV
SEQ ID NO: 698
VAQNSVGEITVH
SEQ ID NO: 699
AQNSVGEITVHA
SEQ ID NO: 700
QNSVGEITVHAK
SEQ ID NO: 701
NSVGEITVHAKL
SEQ ID NO: 702
SVGEITVHAKLT
SEQ ID NO: 703
VGEITVHAKLTV
SEQ ID NO: 704
GEITVHAKLTVL
SEQ ID NO: 705
EITVHAKLTVLR
SEQ ID NO: 706
ITVHAKLTVLRE

In some embodiments, the peptide comprises an amino acid sequence selected from 13 consecutive residues of SEQ ID NO: 442, or from the group consisting of the below:

SEQ ID NO: 707
EEPPRFIKEPKDQ
SEQ ID NO: 708
EPPRFIKEPKDQI
SEQ ID NO: 709
PPRFIKEPKDQIG
SEQ ID NO: 710
PRFIKEPKDQIGV
SEQ ID NO: 711
RFIKEPKDQIGVS
SEQ ID NO: 712
FIKEPKDQIGVSG
SEQ ID NO: 713
IKEPKDQIGVSGG
SEQ ID NO: 714
KEPKDQIGVSGGV
SEQ ID NO: 715
EPKDQIGVSGGVA
SEQ ID NO: 716
PKDQIGVSGGVAS
SEQ ID NO: 717
KDQIGVSGGVASF
SEQ ID NO: 718
DQIGVSGGVASFV
SEQ ID NO: 719
QIGVSGGVASFVC
SEQ ID NO: 720
IGVSGGVASFVCQ
SEQ ID NO: 721
GVSGGVASFVCQA
SEQ ID NO: 722
VSGGVASFVCQAT
SEQ ID NO: 723
SGGVASFVCQATG
SEQ ID NO: 724
GGVASFVCQATGD
SEQ ID NO: 725
GVASFVCQATGDP
SEQ ID NO: 726
VASFVCQATGDPK
SEQ ID NO: 727
ASFVCQATGDPKP
SEQ ID NO: 728
SFVCQATGDPKPR
SEQ ID NO: 729
FVCQATGDPKPRV
SEQ ID NO: 730
VCQATGDPKPRVT
SEQ ID NO: 731
CQATGDPKPRVTW
SEQ ID NO: 732
QATGDPKPRVTWN
SEQ ID NO: 733
ATGDPKPRVTWNK
SEQ ID NO: 734
TGDPKPRVTWNKK
SEQ ID NO: 735
GDPKPRVTWNKKG
SEQ ID NO: 736
DPKPRVTWNKKGK
SEQ ID NO: 737
PKPRVTWNKKGKK
SEQ ID NO: 738
KPRVTWNKKGKKV
SEQ ID NO: 739
PRVTWNKKGKKVN
SEQ ID NO: 740
RVTWNKKGKKVNS
SEQ ID NO: 741
VTWNKKGKKVNSQ
SEQ ID NO: 742
TWNKKGKKVNSQR
SEQ ID NO: 743
WNKKGKKVNSQRF
SEQ ID NO: 744
NKKGKKVNSQRFE
SEQ ID NO: 745
KGKKVNSQRFET
SEQ ID NO: 746
KGKKVNSQRFETI
SEQ ID NO: 747
GKKVNSQRFETIE
SEQ ID NO: 748
KKVNSQRFETIEF
SEQ ID NO: 749
KVNSQRFETIEFD
SEQ ID NO: 750
VNSQRFETIEFDE
SEQ ID NO: 751
NSQRFETIEFDES
SEQ ID NO: 752
SQRFETIEFDESA
SEQ ID NO: 753
QRFETIEFDESAG
SEQ ID NO: 754
RFETIEFDESAGA
SEQ ID NO: 755
FETIEFDESAGAV
SEQ ID NO: 756
ETIEFDESAGAVL
SEQ ID NO: 757
TIEFDESAGAVLR
SEQ ID NO: 758
IEFDESAGAVLRI
SEQ ID NO: 759
EFDESAGAVLRIQ
SEQ ID NO: 760
FDESAGAVLRIQP
SEQ ID NO: 761
DESAGAVLRIQPL
SEQ ID NO: 762
ESAGAVLRIQPLR
SEQ ID NO: 763
SAGAVLRIQPLRT
SEQ ID NO: 764
AGAVLRIQPLRTP
SEQ ID NO: 765
GAVLRIQPLRTPR
SEQ ID NO: 766
AVLRIQPLRTPRD
SEQ ID NO: 767
VLRIQPLRTPRDE
SEQ ID NO: 768
LRIQPLRTPRDEN
SEQ ID NO: 769
RIQPLRTPRDENV
SEQ ID NO: 770
IQPLRTPRDENVY
SEQ ID NO: 771
QPLRTPRDENVYE
SEQ ID NO: 772
PLRTPRDENVYEC
SEQ ID NO: 773
LRTPRDENVYECV
SEQ ID NO: 774
RTPRDENVYECVA
SEQ ID NO: 775
TPRDENVYECVAQ
SEQ ID NO: 776
PRDENVYECVAQN
SEQ ID NO: 777
RDENVYECVAQNS
SEQ ID NO: 778
DENVYECVAQNSV
SEQ ID NO: 779
ENVYECVAQNSVG
SEQ ID NO: 780
NVYECVAQNSVGE
SEQ ID NO: 781
VYECVAQNSVGEI
SEQ ID NO: 782
YECVAQNSVGEIT
SEQ ID NO: 783
ECVAQNSVGEITV
SEQ ID NO: 784
CVAQNSVGEITVH
SEQ ID NO: 785
VAQNSVGEITVHA
SEQ ID NO: 786
AQNSVGEITVHAK
SEQ ID NO: 787
QNSVGEITVHAKL
SEQ ID NO: 788
NSVGEITVHAKLT
SEQ ID NO: 789
SVGEITVHAKLTV
SEQ ID NO: 790
VGEITVHAKLTVL
SEQ ID NO: 791
GEITVHAKLTVLR
SEQ ID NO: 792
EITVHAKLTVLRE

In some embodiments, the peptide comprises an amino acid sequence selected from 14 consecutive residues of SEQ ID NO: 442, or from the group consisting of the below:

SEQ ID NO: 793
EEPPRFIKEPKDQI
SEQ ID NO: 794
EPPRFIKEPKDQIG
SEQ ID NO: 795
PPRFIKEPKDQIGV
SEQ ID NO: 796
PRFIKEPKDQIGVS
SEQ ID NO: 797
RFIKEPKDQIGVSG
SEQ ID NO: 798
FIKEPKDQIGVSGG
SEQ ID NO: 799
IKEPKDQIGVSGGV
SEQ ID NO: 800
KEPKDQIGVSGGVA
SEQ ID NO: 801
EPKDQIGVSGGVAS
SEQ ID NO: 802
PKDQIGVSGGVASF
SEQ ID NO: 803
KDQIGVSGGVASFV
SEQ ID NO: 804
DQIGVSGGVASFVC
SEQ ID NO: 805
QIGVSGGVASFVCQ
SEQ ID NO: 806
IGVSGGVASFVCQA
SEQ ID NO: 807
GVSGGVASFVCQAT
SEQ ID NO: 808
VSGGVASFVCQATG
SEQ ID NO: 809
SGGVASFVCQATGD
SEQ ID NO: 810
GGVASFVCQATGDP
SEQ ID NO: 811
GVASFVCQATGDPK
SEQ ID NO: 812
VASFVCQATGDPKP
SEQ ID NO: 813
ASFVCQATGDPKPR
SEQ ID NO: 814
SFVCQATGDPKPRV
SEQ ID NO: 815
FVCQATGDPKPRVT
SEQ ID NO: 816
VCQATGDPKPRVTW
SEQ ID NO: 817
CQATGDPKPRVTWN
SEQ ID NO: 818
QATGDPKPRVTWNK
SEQ ID NO: 819
ATGDPKPRVTWNKK
SEQ ID NO: 820
TGDPKPRVTWNKKG
SEQ ID NO: 821
GDPKPRVTWNKKGK
SEQ ID NO: 822
DPKPRVTWNKKGKK
SEQ ID NO: 823
PKPRVTWNKKGKKV
SEQ ID NO: 824
KPRVTWNKKGKKVN
SEQ ID NO: 825
PRVTWNKKGKKVNS
SEQ ID NO: 826
RVTWNKKGKKVNSQ
SEQ ID NO: 827
VTWNKKGKKVNSQR
SEQ ID NO: 828
TWNKKGKKVNSQRF
SEQ ID NO: 829
WNKKGKKVNSQRFE
SEQ ID NO: 830
NKKGKKVNSQRFET
SEQ ID NO: 831
KKGKKVNSQRFETI
SEQ ID NO: 832
KGKKVNSQRFETIE
SEQ ID NO: 833
GKKVNSQRFETIEF
SEQ ID NO: 834
KKVNSQRFETIEFD
SEQ ID NO: 835
KVNSQRFETIEFDE
SEQ ID NO: 836
VNSQRFETIEFDES
SEQ ID NO: 837
NSQRFETIEFDESA
SEQ ID NO: 838
SQRFETIEFDESAG
SEQ ID NO: 839
QRFETIEFDESAGA
SEQ ID NO: 840
RFETIEFDESAGAV
SEQ ID NO: 841
FETIEFDESAGAVL
SEQ ID NO: 842
ETIEFDESAGAVLR
SEQ ID NO: 843
TIEFDESAGAVLRI
SEQ ID NO: 844
IEFDESAGAVLRIQ
SEQ ID NO: 845
EFDESAGAVLRIQP
SEQ ID NO: 846
FDESAGAVLRIQPL
SEQ ID NO: 847
DESAGAVLRIQPLR
SEQ ID NO: 848
ESAGAVLRIQPLRT
SEQ ID NO: 849
SAGAVLRIQPLRTP
SEQ ID NO: 850
AGAVLRIQPLRTPR
SEQ ID NO: 851
GAVLRIQPLRTPRD
SEQ ID NO: 852
AVLRIQPLRTPRDE
SEQ ID NO: 853
VLRIQPLRTPRDEN
SEQ ID NO: 854
LRIQPLRTPRDENV
SEQ ID NO: 855
RIQPLRTPRDENVY
SEQ ID NO: 856
IQPLRTPRDENVYE
SEQ ID NO: 857
QPLRTPRDENVYEC
SEQ ID NO: 858
PLRTPRDENVYECV
SEQ ID NO: 859
LRTPRDENVYECVA
SEQ ID NO: 860
RTPRDENVYECVAQ
SEQ ID NO: 861
TPRDENVYECVAQN
SEQ ID NO: 862
PRDENVYECVAQNS
SEQ ID NO: 863
RDENVYECVAQNSV
SEQ ID NO: 864
DENVYECVAQNSVG
SEQ ID NO: 865
ENVYECVAQNSVGE
SEQ ID NO: 866
NVYECVAQNSVGEI
SEQ ID NO: 867
VYECVAQNSVGEIT
SEQ ID NO: 868
YECVAQNSVGEITV
SEQ ID NO: 869
ECVAQNSVGEITVH
SEQ ID NO: 870
CVAQNSVGEITVHA
SEQ ID NO: 871
VAQNSVGEITVHAK
SEQ ID NO: 872
AQNSVGEITVHAKL
SEQ ID NO: 873
QNSVGEITVHAKLT
SEQ ID NO: 874
NSVGEITVHAKLTV
SEQ ID NO: 875
SVGEITVHAKLTVL
SEQ ID NO: 876
VGEITVHAKLTVLR
SEQ ID NO: 877
GEITVHAKLTVLRE

In some embodiments, the disclosed peptide further comprises a blood brain barrier penetrating sequence. For example, cell-penetrating peptides (CPPs) are a group of peptides, which have the ability to cross cell membrane bilayers. CPPs themselves can exert biological activity and can be formed endogenously. Fragmentary studies demonstrate their ability to enhance transport of different cargoes across the blood-brain barrier (BBB). The cellular internalization sequence can be any cell-penetrating peptide sequence capable of penetrating the BBB. Non-limiting examples of CPPs include Polyarginine (e.g., R₉), Antennapedia sequences, TAT, HIV-Tat, Penetratin, Antp-3A (Antp mutant), Buforin II, Transportan, MAP (model amphipathic peptide), K-FGF, Ku70, Prion, pVEC, Pep-1, SynB1, Pep-7, HN-1, BGSC (Bis-Guanidinium-Spermidine-Cholesterol, and BGTC (Bis-Guanidinium-Tren-Cholesterol) (see Table 1).

TABLE 1
Cell Internalization Transporters

| Name | Sequence | SEQ ID NO |
|---|---|---|
| Polyarginine | RRRRRRRRR | SEQ ID NO: 878 |
| Antp | RQPKIWFPNRRKPWKK | SEQ ID NO: 879 |
| HIV-Tat | GRKKRRQRPPQ | SEQ ID NO: 880 |
| Penetratin | RQIKIWFQNRRMKWKK | SEQ ID NO: 881 |
| Antp-3A | RQIAIWFQNRRMKWAA | SEQ ID NO: 882 |
| Tat | RKKRRQRRR | SEQ ID NO: 883 |
| Buforin II | TRSSRAGLQFPVGRVHRLLRK | SEQ ID NO: 884 |
| Transportan | GWTLNSAGYLLGKINKALAAL | SEQ ID NO: 885 |
| | AKKIL | |
| model | KLALKLALKALKAALKLA | SEQ ID NO: 886 |
| amphipathic | | |
| peptide | | |
| (MAP) | | |
| K-FGF | AAVALLPAVLLALLAP | SEQ ID NO: 887 |
| Ku70 | VPMLK- PMLKE | SEQ ID NO: 888 |
| Prion | MANLGYWLLALFVTMWTDVGL | SEQ ID NO: 889 |
| | CKKRPKP | |
| pVEC | LLIILRRRIRKQAHAHSK | SEQ ID NO: 890 |
| Pep-1 | KETWWETWWTEWSQPKKKRKV | SEQ ID NO: 891 |
| SynB1 | RGGRLSYSRRRFSTSTGR | SEQ ID NO: 892 |
| Pep-7 | SDLWEMMMVSLACQY | SEQ ID NO: 893 |
| HN-1 | TSPLNIHNGQKL | SEQ ID NO: 894 |
| Tat | GRKKRRQRRRPQ | SEQ ID NO: 895 |
| Tat | RKKRRQRRRC | SEQ ID NO: 896 |

Therefore, in some embodiments, the disclosed peptide is a fusion protein, e.g., containing the APP-binding domain of PTPσ, the PTPσ-binding domain of APP, or a combination thereof, and a CPP. Fusion proteins, also known as chimeric proteins, are proteins created through the joining of two or more genes, which originally coded for separate proteins. Translation of this fusion gene results in a single polypeptide with function properties derived from each of the original proteins. Recombinant fusion proteins can be created artificially by recombinant DNA technology for use in biological research or therapeutics.

In some embodiments, linker (or “spacer”) peptides are also added which make it more likely that the proteins fold independently and behave as expected. Linkers in protein or peptide fusions are sometimes engineered with cleavage sites for proteases or chemical agents which enable the liberation of the two separate proteins. This technique is often used for identification and purification of proteins, by fusing a GST protein, FLAG peptide, or a hexa-his peptide (aka: a 6×his-tag) which can be isolated using nickel or cobalt resins (affinity chromatography). Chimeric proteins can also be manufactured with toxins or antibodies attached to them in order to study disease development.

Compositions that Restore Molecular Balance of CS and HS in the Perineuronal Space:

Chondroitin sulfates (CS) and heparin or its analog heparan sulfates (HS) are two main classes of glycosaminoglycans (GAGs) in the brain that are sensed by neurons via Receptor Protein Tyrosine⁸. The ratio of CS and HS therefore affects the downstream effects of PTPσ, because CS and HS compete to interact with the receptor yet lead to opposite signaling and neuronal responses (such as neurite regeneration). CS increases but HS decreases APP β-cleavage products (Example 2). Therefore, methods involving administering to the subject a composition that restore the physiological molecular CS/HS balance may be used to treat and prevent aforementioned neurodegenerative diseases. These therapies could be applied alternatively or in addition to the polypeptides listed above. In some embodiments, administering HS, or its analog heparin, or their mimetics modified to reduce anti-coagulant effect, with a saccharide chain length of 17, 18, 19, 20, 21, 22, 23, 24 units or longer, could assist in restoring the physiological molecular CS/HS balance. In some embodiments, the balance is restored by administering enzymes that digest CS (such as ChABC) or prevent the degradation of HS (such as Heparanase inhibitors PI-88, OGT 2115, or PG545). Alternatively or in addition, agents that mimic the HS/heparin effect of PTPσ clustering⁸, such as multivalent antibodies, could be administered.

Pharmaceutical Compositions

The peptides disclosed can be used therapeutically in combination with a pharmaceutically acceptable carrier. Pharmaceutical carriers suitable for administration of the compounds provided herein include any such carriers known to those skilled in the art to be suitable for the particular mode of administration. The carrier would naturally be selected to minimize any degradation of the active ingredient and to minimize any adverse side effects in the subject, as would be well known to one of skill in the art.

In some embodiments, the peptides described above are formulated into pharmaceutical compositions using techniques and procedures well known in the art (See, e.g., Ansel, Introduction to Pharmaceutical Dosage Forms, 4th Edition, 1985, 126).

Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension.

Dosage forms or compositions containing active ingredient in the range of 0.005% to 100% with the balance made up from non-toxic carrier may be prepared. Methods for preparation of these compositions are known to those skilled in the art. The contemplated compositions may contain 0.001%-100% active ingredient, or in one embodiment 0.1-95%.

Methods of Screening

Also disclosed are methods of screening for candidate compounds that slow, stop, reverse, or prevent neurodegeneration.

### Methods of Screening Based on APP-PTPσ Binding:

In some embodiments, the method comprising providing a sample comprising APP and PTPσ in an environment permissive for APP-PTPσ binding, contacting the sample with a candidate compound, and assaying the sample for APP-PTPσ binding, wherein a decrease in APP-PTPσ binding compared to control values is an indication that the candidate agent is effective to slow, stop, reverse, or prevent neurodegeneration.

The binding of PTPσ to APP can be detected using routine methods that do not disturb protein binding.

In some embodiments, the binding of PTPσ to APP can be detected using immunodetection methods. The steps of various useful immunodetection methods have been described in the scientific literature, such as, e.g., Maggio et al., Enzyme-Immunoassay, (1987) and Nakamura, et al., Enzyme Immunoassays: Heterogeneous and Homogeneous Systems, Handbook of Experimental Immunology, Vol. 1: Immunochemistry, 27.1-27.20 (1986), each of which is incorporated herein by reference in its entirety and specifically for its teaching regarding immunodetection methods. Immunoassays, in their most simple and direct sense, are binding assays involving binding between antibodies and antigen. Examples of immunoassays are enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

The methods can be cell-based or cell-free assays.

In some embodiments, the binding between PTPσ and APP can be detected using fluorescence activated cell sorting (FACS). For example, disclosed are cell lines transfected with of PTPσ and APP fused to fluorescent proteins. These cell lines can facilitate high-throughput screens for biologically expressed and chemically synthesized molecules that disrupt the binding between PTPσ and APP.

In some embodiments, the binding between PTPσ and APP can be detected in a cell-free setting where one of these two binding partners is purified and immobilized/captured through covalent or non-covalent bond to a solid surface or beads, while the other binding partner is allowed to bind in the presence of biologically expressed and chemically synthesized molecules to screen candidate agents for their efficacies in dissociating APP-PTPσ interaction.

In some embodiments, the binding between PTPσ and APP can be detected in a setting where cell membrane preparations extracted from fresh rodent brain homogenates (containing both APP and PTPσ) are contacted with biologically expressed and chemically synthesized molecules. Subsequently, one of the binding partners is immunoprecipitated and the binding or co-immunoprecipitation of the other binding partner is detected using its specific antibody.

A candidate agent that decreases or abolishes APP-PTPσ binding in a disclosed method herein has the potential to slow, stop, reverse, or prevent neurodegeneration.

### Methods of Screening Based on APP Amyloidogenic Processing:

In some embodiments, the method comprising contacting/incubating a candidate compound with cell membrane preparations extracted from fresh rodent brain homogenates, wherein a decrease in APP β- and/or γ-cleavage products is an indication that the candidate agent has the potential to slow, stop, reverse, or prevent neurodegeneration. APP β- and/or γ-cleavage products can be detected by routine biochemical methods such as Western blot analysis, ELISA, and immnuopurification.

### Libraries of Molecules and Compounds:

In general, candidate agents can be identified from large libraries of natural products or synthetic (or semi-synthetic) extracts or chemical libraries according to methods known in the art. Those skilled in the field of drug discovery and development will understand that the precise source of test extracts or compounds is not critical to the screening procedure(s) used.

Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available, e.g., from purveyors of chemical libraries including but not limited to ChemBridge Corporation (16981 Via Tazon, Suite G, San Diego, Calif., 92127, USA, www.chembridge.com); ChemDiv (6605 Nancy Ridge Drive, San Diego, Calif. 92121, USA); Life Chemicals (1103 Orange Center Road, Orange, Conn. 06477); Maybridge (Trevillett, Tintagel, Cornwall PL34 0HW, UK).

Alternatively, libraries of natural compounds in the form of bacterial, fungal, plant, and animal extracts are commercially available from a number of sources, including O2H, (Cambridge, UK), MerLion Pharmaceuticals Pte Ltd (Singapore Science Park II, Singapore 117528) and Galapagos NV (Generaal De Wittelaan L11 A3, B-2800 Mechelen, Belgium).

In addition, natural and synthetically produced libraries are produced, if desired, according to methods known in the art, e.g., by standard extraction and fractionation methods or by standard synthetic methods in combination with solid phase organic synthesis, micro-wave synthesis and other rapid throughput methods known in the art to be amenable to making large numbers of compounds for screening purposes. Furthermore, if desired, any library or compound, including sample format and dissolution is readily modified and adjusted using standard chemical, physical, or biochemical methods.

Candidate agents encompass numerous chemical classes, but are most often organic molecules, e.g., small organic compounds having a molecular weight of more than 100 and less than about 2,500 Daltons, or, in some embodiments, having a molecular weight of more than 100 and less than about 5,000 Daltons. Candidate agents can include functional groups necessary for structural interaction with proteins, particularly hydrogen bonding, and typically include at least an amine, carbonyl, hydroxyl or carboxyl group, for example, at least two of the functional chemical groups. The candidate agents often contain cyclical carbon or heterocyclic structures and/or aromatic or polyaromatic structures substituted with one or more of the above functional groups.

In some embodiments, the candidate agents are proteins. In some aspects, the candidate agents are naturally occurring proteins or fragments of naturally occurring proteins. Thus, for example, cellular extracts containing proteins, or random or directed digests of proteinaceous cellular extracts, can be used. In this way libraries of procaryotic and eucaryotic proteins can be made for screening using the methods herein. The libraries can be bacterial, fungal, viral, and vertebrate proteins, and human proteins.

Methods of Treatment

Disclosed herein are methods for treating neurodegenerative diseases that involve β-amyloid pathologies and/or Tau pathologies, including but not limited to Alzheimer's disease, Lewy body dementia, frontotemporal dementia, cerebral amyloid angiopathy, primary age-related tauopathy, chronic traumatic encephalopathy, Parkinson's disease, postencephalitic parkinsonism, Huntington's disease, amyolateral sclerosis, Pick's disease, progressive supranuclear palsy, corticobasal degeneration, Lytico-Bodig disease, ganglioglioma and gangliocytoma, subacute sclerosing panencephalitis, Hallervorden-Spatz disease, and/or Creutzfeldt-Jakob disease.

These peptides, compositions, and methods may also be used to prevent these neurodegenerative diseases in populations at risk, such as people with Down syndrome and those suffered from brain injuries or cerebral ischemia, as well as the aging population.

In some embodiments, these methods involve disrupting the binding between PTPσ and APP, preventing β-amyloidogenic processing of APP without affecting other major substrates of β- and γ-secretases. For example, the methods can involve administering to a subject a peptide disclosed herein. In other embodiments, monoclonal antibodies could be formed against the IG1 domain of PTPσ or a fragment thereof, a fragment between the E1 and E2 domain of the APP695 isoform, or both, and these antibodies, or fragments thereof, could be administered to the subject.

Chondroitin sulfates (CS) and heparin or its analog heparan sulfates (HS) are two main classes of glycosaminoglycans (GAGs) in the brain that are “sensed” by neurons via Receptor Protein Tyrosine⁸. The ratio of CS and HS therefore affects the downstream effects of PTPσ, because CS and HS compete to interact with the receptor yet lead to opposite signaling and neuronal responses (such as neurite regeneration). CS increases but HS decreases APP β-cleavage products (Example 2). Therefore, in some embodiments, the methods involve administering to the subject a composition, which restores the physiological molecular CS/HS balance, may be used to treat and prevent aforementioned neurodegenerative diseases. These therapies could be applied alternatively or in addition to the polypeptides listed above. In some embodiments, administering HS, or its analog heparin, or their mimetics modified to reduce anti-coagulant effects, with a saccharide chain length of 17, 18, 19, 20, 21, 22, 23, 24 units or longer, could assist in restoring the physiological molecular CS/HS balance. In some embodiments, the balance is restored by administering enzymes that digest CS (such as Chondroitinase ABC) or prevent the degradation of HS (such as Heparanase inhibitors PI-88, OGT 2115, or PG545). Alternatively or in addition, agents that mimic the HS/heparin effect of PTPσ clustering⁸, such as multivalent antibodies, could be administered.

In some embodiments, the method involves administering a composition described herein in a dose equivalent to parenteral administration of about 0.1 ng to about 100 g per kg of body weight, about 10 ng to about 50 g per kg of body weight, about 100 ng to about 1 g per kg of body weight, from about 1 μg to about 100 mg per kg of body weight, from about 1 μg to about 50 mg per kg of body weight, from about 1 mg to about 500 mg per kg of body weight; and from about 1 mg to about 50 mg per kg of body weight. Alternatively, the amount of composition administered to achieve a therapeutic effective dose is about 0.1 ng, 1 ng, 10 ng, 100 ng, 1 μg, 10 μg, 100 μg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 30 mg, 40 mg, 50 mg, 60 mg, 70 mg, 80 mg, 90 mg, 100 mg, 500 mg per kg of body weight or greater.

A number of embodiments of the invention have been described. Nevertheless, it will be understood that various modifications may be made without departing from the spirit and scope of the invention. Accordingly, other embodiments are within the scope of the following claims.

### Examples

### Example 1: Alzheimer's Disease Pathogenesis is Dependent on Neuronal Receptor PTPσ

Methods and Materials

Mouse lines: Mice were maintained under standard conditions approved by the Institutional Animal Care and Use Committee. Wild type and PTPσ-deficient mice of Balb/c background were provided by Dr. Michel L. Tremblay⁹. Homozygous TgAPP-SwDI mice, C57BL/6-Tg(Thy1-APPSwDutIowa)BWevn/Mmjax, stock number 007027, were from the Jackson Laboratory. These mice express human APP transgene harboring Swedish, Dutch, and Iowa mutations, and were bred with Balb/c mice heterozygous for the PTPσ gene to generate bigenic mice heterozygous for both TgAPP-SwDI and PTPσ genes, which are hybrids of 50% C57BL/6J and 50% Balb/c genetic background. These mice were further bred with Balb/c mice heterozygous for the PTPσ gene. The offspring from this mating are used in experiments, which include littermates of the following genotypes: TgAPP-SwDI(+/−)PTPσ(+/+), mice heterozygous for TgAPP-SwDI transgene with wild type PTPα; TgAPP-SwDI(+/−)PTPσ(−/−), mice heterozygous for TgAPP-SwDI transgene with genetic depletion of PTPσ; TgAPP-SwDI(−/−) PTPσ(+/+), mice free of TgAPP-SwDI transgene with wild type PTPσ. Both TgAPP-SwDI(−/−) PTPσ(+/+) and Balb/c PTPσ(+/+) are wild type mice but with different genetic background. Heterozygous TgAPP-SwInd (J20) mice, 6.Cg-Tg(PDGFB-APPSwInd)20Lms/2Mmjax, were provided by Dr. Lennart Mucke. These mice express human APP transgene harboring Swedish and Indiana mutations, and were bred with the same strategy as described above to obtain mice with genotypes of TgAPP-SwInd (+/−)PTPσ(+/+) and TgAPP-SwInd (+/−)PTPσ(−/−).

Antibodies:

| Primary Antibodies | Application | Clone | Catalog # | Supplier |
|---|---|---|---|---|
| Mouse anti-Actin | WB | AC-40 | A700 | Sigma-Aldrich |
| Rabbit anti-APH1 | WB | | PAS-20318 | Thermo Scientific |
| Rabbit anti-APP C-term | WB, IP, IHC | Y188 | NIB 110-55461 | Novus Biologicals |
| Mouse anti-murine Ap, 1-16 | WB, IP | M3.2 | 805701 | Biolegend |
| Mouse anti-human A13. 1-16 | WB, IP, IHC, ELISA | 6E10 | 803001 | Biolegend |
| Mouse anti-A13, 17-24 | WB, IHC | 4G8 | SIG-39220 | Biolegend |
| Mouse HRP-conjugated anti-A13 1-40 | ELISA | 11A50-B10 | SIG-39146 | Biolegend |
| Mouse HRP-conjugated anti-A13 1-42 | ELISA | 12F4 | 805507 | Biolegend |
| Rabbit anti-BACE 1 C-Term, B690 | WB | | PRB-617C | Covance |
| Guinea Pig anti-BACE 1 C-Term | IP | | 840201 | Biolegend |
| Chicken anti-GFAP | IHC | | ab4674 | Abcam |
| Rabbit anti-Neuregulin | WB | | sc-348 | Santa Cruz |
| | | | | Biotechnology |
| Rabbit anti-Nicastrin | WB | | 5665 | Cell Signaling |
| Rabbit anti-Notch NICD (va11744) | WB | | 4147 | Cell Signaling |
| Rabbit anti-Notch (C-20) | WB | | sc-6014R | Santa Cruz |
| | | | | Biotechnology |
| Rabbit anti-PEN2 | WB | | 8598 | Cell Signaling |
| Rabbit anti-Presenilin 1/2 NTF | WB | | 840201 | Abcam |
| Rabbit anti-Presenilin 1 CTF | WB | | 5643 | Cell Signaling |
| Rabbit anti-Presenilin 2 CTF | WB | | 9979 | Cell Signaling |
| Mouse anti-PTP u ICD | WB, IHC | 17G7.2 | MM-002-P | Medimabs |
| Mouse anti-PTP u ECD | WB | | ab55640 | Abcam |
| Rabbit anti-Synaptophysin | IHC | | AB9272 | Millipore |
| Mouse anti-Tau | WB, IHC | Tau-5 | MAB361 | Millipore |
| Mouse anti-Tau | IHC | Tau-46 | 4019 | Cell Signaling |
| Secondary and Tertiary Antibodies | Application | Clone | Catalog # | Supplier |
| Goat anti-mouse IgG HRP-conjugated | WB | 7076S | | Cell Signaling |
| Goat anti-rabbit IgG HRP-conjugated | WB | 7074S | | Cell Signaling |
| Goat anti-mouse IgG Alexa488 | IHC | A-11001 | | Invitrogen |
| Donkey anti-goat IgG Alexa488 | IHC | A-11055 | | Invitrogen |
| Chicken anti-rabbit IgG CF568 | IHC | 5AB4600426 | | Sigma-Aldrich |
| Donkey anti-chicken IgG Cy3 | IHC | 703-165-155 | | JacksonImmunoResearch |

Immunohistochemistry: Adult rat and mice were perfused intracardially with fresh made 4% paraformaldehyde in cold phosphate-buffered saline (PBS). The brains were collected and post-fixed for 2 days at 4° C. Paraffin embedded sections of 10 μM thickness were collected for immunostaining. The sections were deparaffinized and sequentially rehydrated. Antigen retrieval was performed at 100° C. in Tris-EDTA buffer (pH 9.0) for 50 min. Sections were subsequently washed with distilled water and PBS, incubated at room temperature for 1 hour in blocking buffer (PBS, with 5% normal donkey serum, 5% normal goat serum, and 0.2% Triton X-100). Primary antibody incubation was performed in a humidified chamber at 4° C. overnight. After 3 washes in PBS with 0.2% Triton X-100, the sections were then incubated with a mixture of secondary and tertiary antibodies at room temperature for 2 hours. All antibodies were diluted in blocking buffer with concentrations recommended by the manufacturers. Mouse primary antibodies were detected by goat anti-mouse Alexa488 together with donkey anti-goat Alexa488 antibodies; rabbit primary antibodies were detected by chicken anti-rabbit CF568 and donkey anti-chicken Cy3 antibodies; chicken antibody was detected with donkey anti-chicken Cy3 antibody. Sections stained with only secondary and tertiary antibodies (without primary antibodies) were used as negative controls. At last, DAPI (Invitrogen, 300 nM) was applied on sections for nuclear staining. Sections were washed 5 times before mounted in Fluoromount (SouthernBiotech).

Wide field and confocal images were captured using Zeiss Axio Imager M2 and LSM780, respectively. Images are quantified using the Zen 2 Pro software and ImageJ.

Protein extraction, immunoprecipitation, and western blot analysis: For the co-immunoprecipitation of APP and PTPσ, RIPA buffer was used (50 mM Tris-HCl, pH 8.0, 1 mM EDTA, 150 mM NaCl, 1% NP40, 0.1% SDS, 0.5% sodium deoxycholate). For the co-immunoprecipitation of APP and BACE1, NP40 buffer was used (50 mM Tris-HCl, pH 8.0, 1 mM EDTA, 150 mM NaCl, 1% NP40) without or with SDS at concentration of 0.1%, 0.3%, and 0.4%. For total protein extraction and immunopurification of CTFβ, SDS concentration in RIPA buffer was adjusted to 1% to ensure protein extraction from the lipid rafts. Mouse or rat forebrains were homogenized thoroughly on ice in homogenization buffers (as mention above) containing protease and phosphatase inhibitors (Thermo Scientific). For each half of forebrain, buffer volume of at least 5 ml for mouse and 8 ml for rat was used to ensure sufficient detergent/tissue ratio. The homogenates were incubated at 4° C. for 1 hour with gentle mixing, sonicated on ice for 2 minutes in a sonic dismembrator (Fisher Scientific Model 120, with pulses of 50% output, 1 second on and 1 second off), followed with another hour of gentle mixing at 4° C. All samples were used fresh without freezing and thawing.

For co-immunoprecipitation and immunopurification, the homogenates were then centrifuged at 85,000×g for 1 hour at 4° C. and the supernatants were collected. Protein concentration was measured using BCA Protein Assay Kit (Thermo Scientific). 0.5 mg total proteins of brain homogenates were incubated with 5 μg of designated antibody and 30 μl of Protein-A sepharose beads (50% slurry, Roche), in a total volume of 1 ml adjusted with RIPA buffer. Samples were gently mixed at 4° C. overnight. Subsequently, the beads were washed 5 times with cold immunoprecipitation buffer. Samples were then incubated in Laemmli buffer with 100 mM of DTT at 75° C. for 20 minutes and subjected to western blot analysis.

For analysis of protein expression level, the homogenates were centrifuged at 23,000×g for 30 min at 4° C. and the supernatants were collected. Protein concentration was measured using BCA Protein Assay Kit (Thermo Scientific). 30 μg of total proteins were subjected to western blot analysis.

Electrophoresis of protein samples was conducted using 4-12% Bis-Tris Bolt Plus Gels, with either MOPS or MES buffer and Novex Sharp Pre-stained Protein Standard (all from Invitrogen). Proteins were transferred to nitrocellulose membrane (0.2 μm pore size, Bio-Rad) and blotted with selected antibodies (see table above) at concentrations suggested by the manufacturers. Primary antibodies were diluted in SuperBlock TBS Blocking Buffer (Thermo Scientific) and incubated with the nitrocellulose membranes at 4° C. overnight; secondary antibodies were diluted in PBS with 5% nonfat milk and 0.2% Tween20 and incubated at room temperature for 2 hours. Membranes were washes 4 times in PBS with 0.2% Tween20 between primary and secondary antibodies and before chemiluminescent detection with SuperSignal West Pico Chemiluminescent Substrate (Thermo Scientific).

Western blot band intensity was quantified by densitometry.

Aβ ELISA assays: Mouse forebrains were thoroughly homogenized in tissue homogenization buffer (2 mM Tris pH 7.4, 250 mM sucrose, 0.5 mM EDTA, 0.5 mM EGTA) containing protease inhibitor cocktail (Roche), followed by centrifugation at 135,000×g (33,500 RPM with SW50.1 rotor) for 1 hour at 4° C. Proteins in the pellets were extracted with formic acid (FA) and centrifuged at 109,000×g (30,100 RPM with SW50.1 rotor) for 1 hour at 4° C. The supernatants were collected and diluted 1:20 in neutralization buffer (1 M Tris base, 0.5 M Na₂HPO₄, 0.05% NaN₃) and subsequently 1:3 in ELISA buffer (PBS with 0.05% Tween-20, 1% BSA, and 1 mM AEBSF). Diluted samples were loaded onto ELISA plates pre-coated with 6E10 antibody (Biolegend) to capture Aβ peptides. Serial dilutions of synthesized human Aβ 1-40 or 1-42 (American Peptide) were loaded to determine a standard curve. Aβ was detected using an HRP labeled antibody for either Aβ 1-40 or 1-42 (see table above). ELISA was developed using TMB substrate (Thermo Scientific) and reaction was stopped with 1N HCl. Plates were read at 450 nm and concentrations of Aβ in samples were determined using the standard curve.

Behavior assays: The Y-maze assay: Mice were placed in the center of the Y-maze and allowed to move freely through each arm. Their exploratory activities were recorded for 5 minutes. An arm entry is defined as when all four limbs are within the arm. For each mouse, the number of triads is counted as “spontaneous alternation”, which was then divided by the number of total arm entries, yielding a percentage score. The novel object test: On day 1, mice were exposed to empty cages (45 cm×24 cm×22 cm) with blackened walls to allow exploration and habituation to the arena. During day 2 to day 4, mice were returned to the same cage with two identical objects placed at an equal distance. On each day mice were returned to the cage at approximately the same time during the day and allowed to explore for 10 minutes. Cages and objects were cleaned with 70% ethanol between each animal. Subsequently, 2 hours after the familiarization session on day 4, mice were put back to the same cage where one of the familiar objects (randomly chosen) was replaced with a novel object, and allowed to explore for 5 minutes. Mice were scored using Observer software (Noldus) on their time duration and visiting frequency exploring either object. Object exploration was defined as facing the object and actively sniffing or touching the object, whereas any climbing behavior was not scored. The discrimination indexes reflecting interest in the novel object is denoted as either the ratio of novel object exploration to total object exploration (NO/NO+FO) or the ratio of novel object exploration to familiar object exploration (NO/FO). All tests and data analyses were conducted in a double-blinded manner.

Statistics: 2-tailed Student's t test was used for two-group comparison. Relationship between two variables was analyzed using linear regression. All error bars show standard error of the means (SEM).

Results

PTPσ is an APP Binding Partner in the Brain.

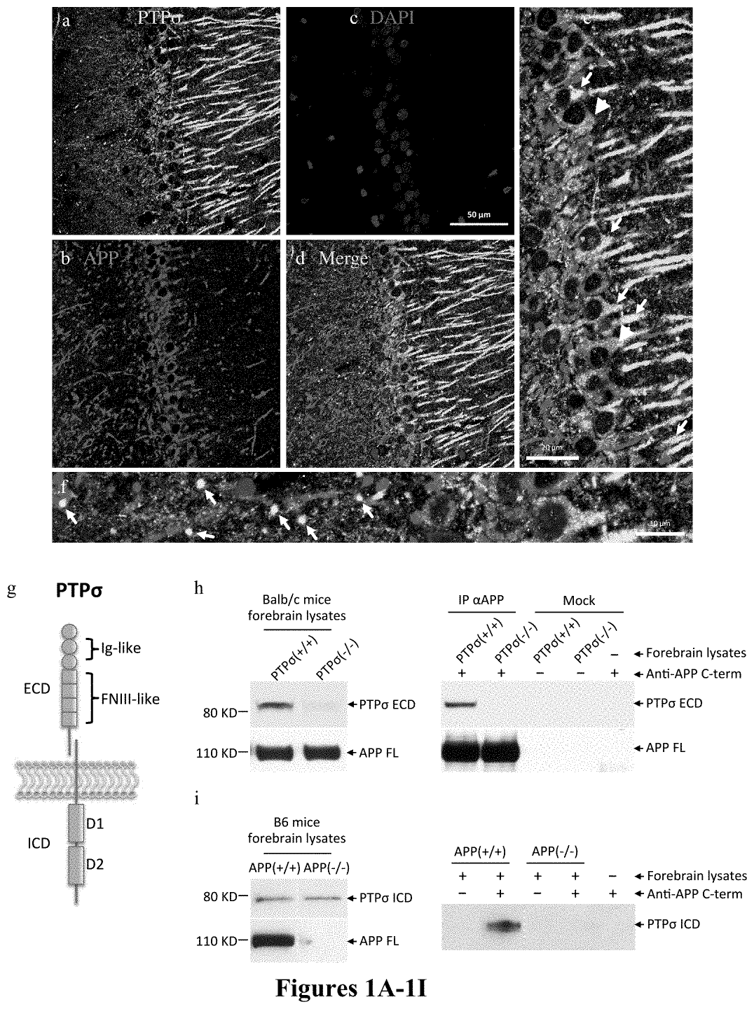

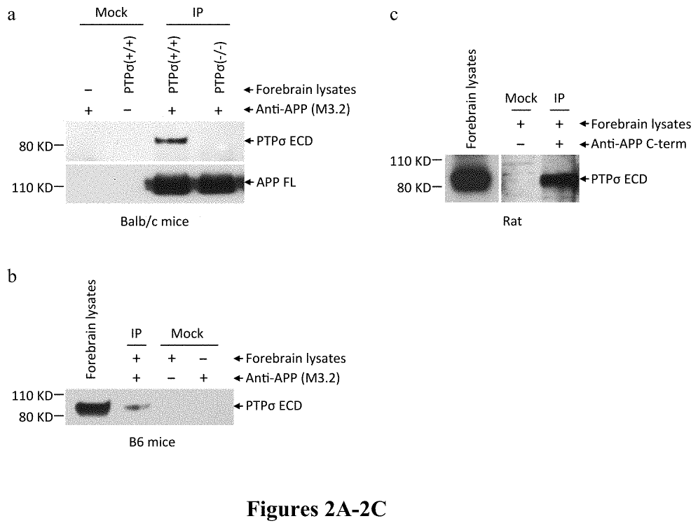

Previously identified as a neuronal receptor of extracellular proteoglycans^{8,10,11} PTPσ is expressed throughout the adult nervous system, most predominantly in the hippocampus^{12,13}, one of earliest affected brain regions in AD. Using immunohistochemistry and confocal imaging, it was found that PTPσ and APP (the precursor of Aβ) colocalize in hippocampal pyramidal neurons of adult rat brains, most intensively in the initial segments of apical dendrites, and in the perinuclear and axonal regions with a punctate pattern (FIGS. 1a-f). To assess whether this colocalization reflects a binding interaction between these two molecules, co-immunoprecipitation experiments were run from brain homogenates. In brains of rats and mice with different genetic background, using various antibodies of APP and PTPσ, a fraction of PTPσ that co-immunoprecipitates with APP was consistently detected, providing evidence of a molecular complex between these two transmembrane proteins (FIGS. 1h, i; FIG. 2).

Genetic Depletion of PTPσ Reduces β-Amyloidogenic Products of APP.

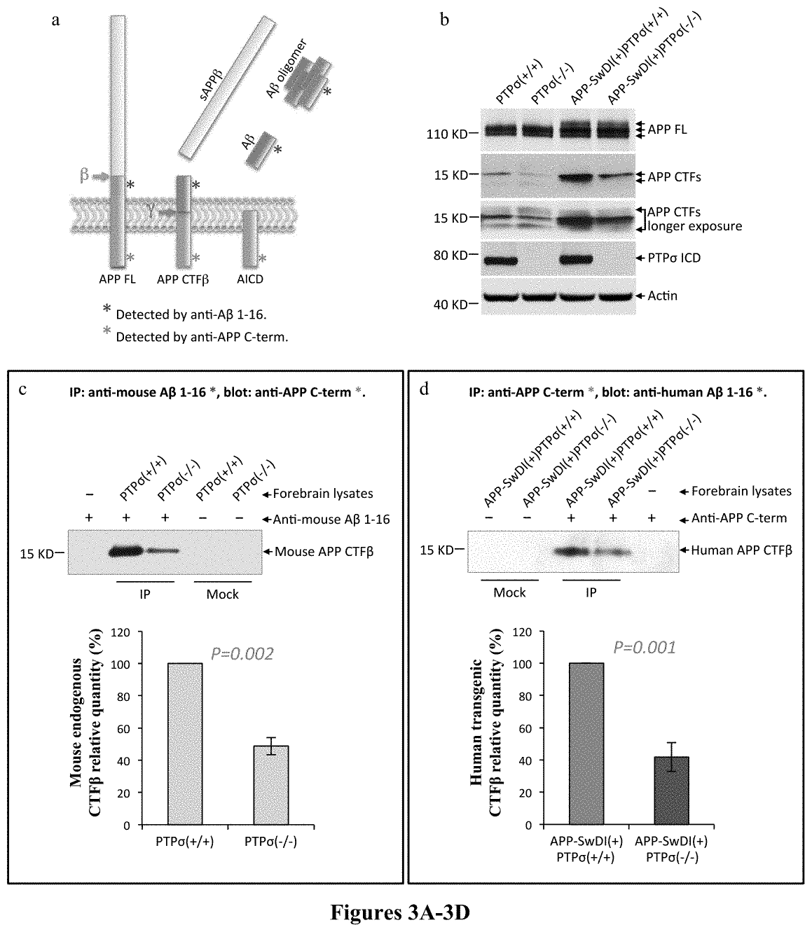

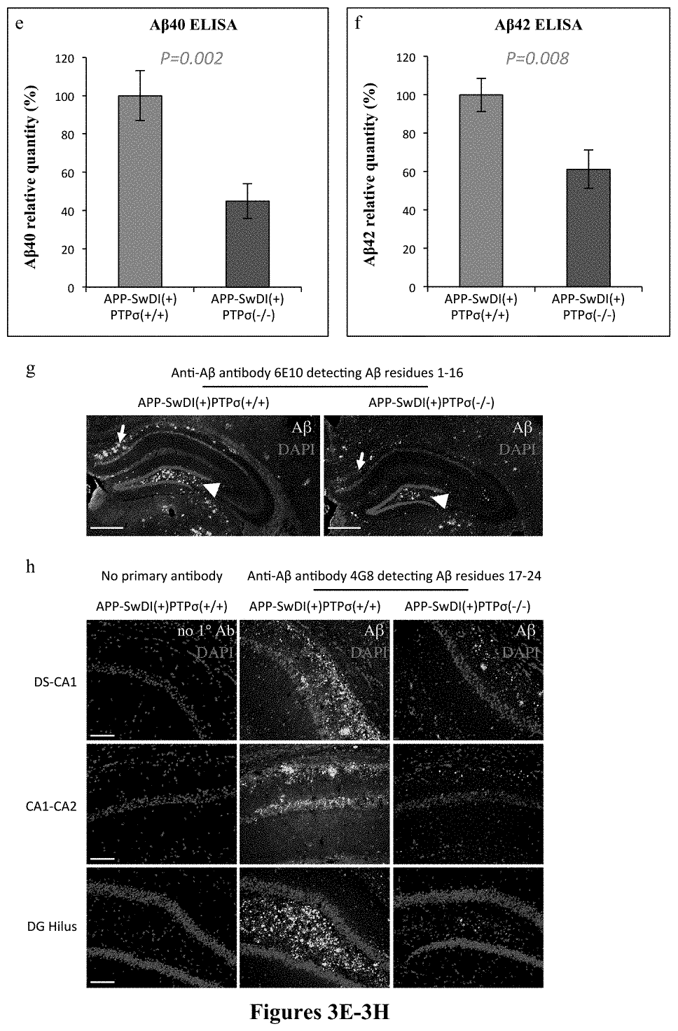

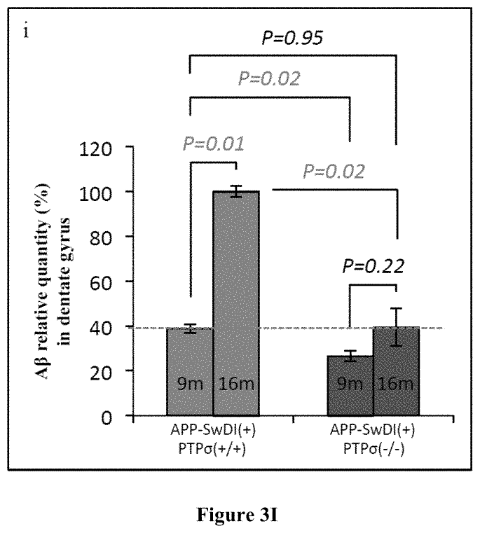

The molecular interaction between PTPσ and APP prompted an investigation on whether PTPσ plays a role in amyloidogenic processing of APP. In neurons, APP is mainly processed through alternative cleavage by either α- or β-secretase. These secretases release the N-terminal portion of APP from its membrane-tethering C-terminal fragment (CTFα or CTFβ, respectively), which can be further processed by the γ-secretase^{14,15} Sequential cleavage of APP by the β- and γ-secretases is regarded as amyloidogenic processing since it produces Aβ peptides¹⁶. When overproduced, the Aβ peptides can form soluble oligomers that trigger ramification of cytotoxic cascades, whereas progressive aggregation of Aβ eventually results in the formation of senile plaques in the brains of AD patients (FIG. 3a). To test the effect of PTPσ in this amyloidogenic processing, the levels of APP β- and γ-cleavage products in mouse brains were analyzed, with or without PTPσ.

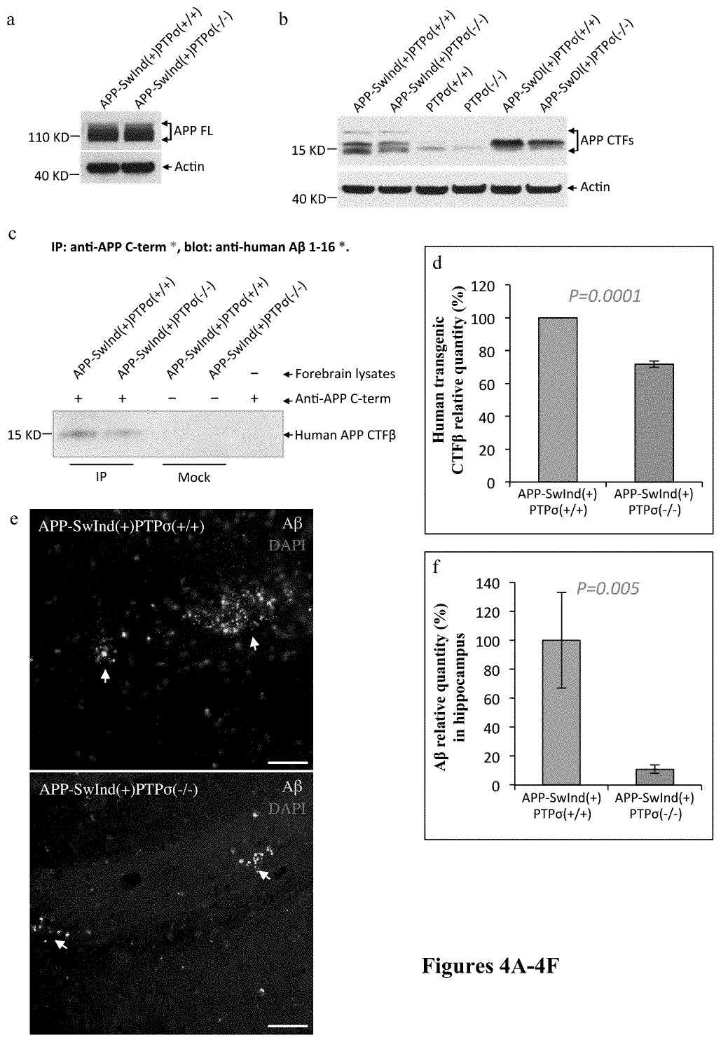

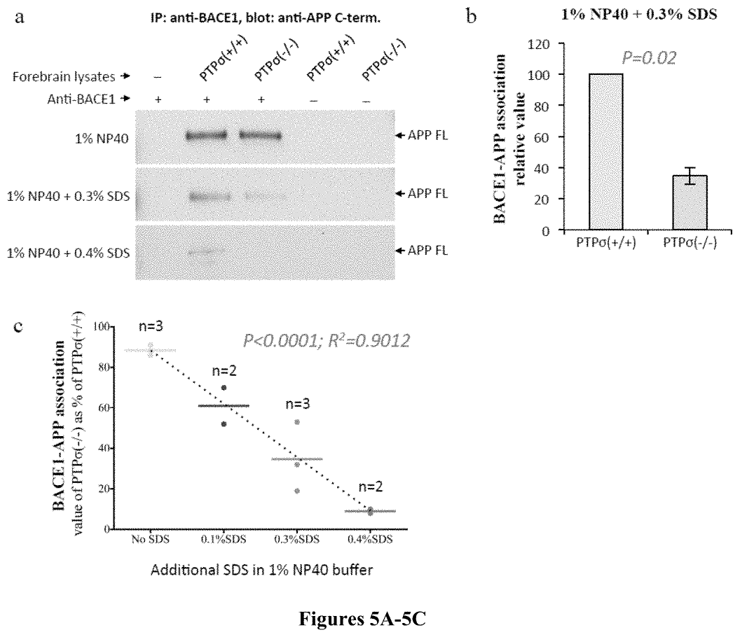

Western blot analysis with protein extracts from mouse brains showed that genetic depletion of PTPσ does not affect the expression level of full length APP (FIG. 3b; FIG. 4a). However, an antibody against the C-terminus of APP detects a band at a molecular weight consistent with CTFβ, which is reduced in PTPσ-deficient mice as compared to their age-sex-matched wild type littermates (FIG. 3b). Additionally, in two AD mouse models expressing human APP genes with amyloidogenic mutations^{17,18}, a similar decrease of an APP CTF upon PTPσ depletion was observed (FIG. 3b; FIG. 4b). The TgAPP-SwDI and TgAPP-SwInd mice, each expressing a human APP transgene harboring the Swedish mutation near the β-cleavage site, were crossed with the PTPσ line to generate offsprings that are heterozygous for their respective APP transgene, with or without PTPσ. Because the Swedish mutation carried by these APP transgenes is prone to β-cleavage, the predominant form of APP CTF in these transgenic mice is predicted to be CTFβ. Thus, the reduction of APP CTF in PTPσ-deficient APP transgenic mice may indicate a regulatory role of PTPσ on CTFβ level. However, since the APP C-terminal antibody used in these experiments can recognize both CTFα and CTFβ, as well as the phosphorylated species of these CTFs (longer exposure of western blots showed multiple CTF bands), judging the identity of the reduced CTF simply by its molecular weight may be inadequate. CTFβ immunopurification was therefore performed with subsequent western blot detection, using an antibody that recognizes CTFβ but not CTFα (FIG. 3c, d; FIG. 4c, d). With this method, we confirmed that PTPσ depletion decreases the level of CTFβ originated from both mouse endogenous and human transgenic APP.

Because CTFβ is an intermediate proteolytic product between β- and γ-cleavage, its decreased steady state level could result from either reduced production by n-cleavage or increased degradation by subsequent γ-secretase cleavage (FIG. 3a). To distinguish between these two possibilities, the level of Aβ peptides was measured, because they are downstream products from CTFβ degradation by γ-cleavage. Using ELISA assays with brain homogenates from the TgAPP-SwDI mice, it was found that PTPσ depletion decreases the levels of Aβ peptides to a similar degree as that of CTFβ (FIG. 3e, f). Consistently, as Aβ peptides gradually aggregate into plaques during aging of the transgenic mice, a substantial decrease of cerebral Aβ deposition was observed in APP transgenic PTPσ-deficient mice as compared to the age-matched APP transgenic littermates expressing wild type PTPσ (FIGS. 3g, h; FIGS. 4e, f). Thus, the concurrent decrease of β- and γ-cleavage products argues against an increased γ-secretase activity, but instead suggests a reduced β-secretase cleavage of APP, which suppresses not only the level of CTFβ but also downstream Aβ production in PTPσ-deficient brains.

Curtailed Progression of β-Amyloidosis in the Absence of PTPσ.

Progressive cerebral Aβ aggregation (β-amyloidosis) is regarded as a benchmark of AD progression. To investigate the effects of PTPσ on this pathological development, Aβ deposits in the brains of 9-month old (mid-aged) and 16-month old (aged) TgAPP-SwDI mice were monitored. At age of 9 to 11 months, Aβ deposits are found predominantly in the hippocampus, especially in the hilus of the dentate gyrus (DG) (FIGS. 3g, h). By 16 months, the pathology spreads massively throughout the entire brain. The propagation of Aβ deposition, however, is curbed by genetic depletion of PTPσ, as quantified using the DG hilus as a representative area (FIG. 3i). Between the ages of 9 and 16 months, the Aβ burden is more than doubled in TgAPP-SwDI mice expressing wild type PTPσ [APP-SwDI(+)PTPσ(+/+)], but only shows marginal increase in the transgenic mice lacking functional PTPσ [APP-SwDI(+)PTPσ(−/−)]. Meanwhile, the Aβ loads measured in 9-month old APP-SwDI(+)PTPσ(+/+) mice are similar to those of 16-month old APP-SwDI(+)PTPσ(−/−) mice (p=0.95), indicating a restraint of disease progression by PTPσ depletion (FIG. 3i).

Decreased BACE1-APP Affinity in PTPσ-Deficient Brains.

Consistent with these observations that suggest a facilitating role of PTPσ in APP β-cleavage, the data further reveal that PTPσ depletion weakens the interaction of APP with BACE1, the β-secretase in the brain. To test the in vivo affinity between BACE1 and APP, co-immunoprecipitation were performed of the enzyme and substrate from mouse brain homogenates in buffers with serially increased detergent stringency. Whereas BACE1-APP association is nearly equal in wild type and PTPσ-deficient brains under mild buffer conditions, increasing detergent stringency in the buffer unveils that the molecular complex is more vulnerable to dissociation in brains without PTPσ (FIG. 5). Thus a lower BACE1-APP affinity in PTPσ-deficient brains may likely be an underlying mechanism for the decreased levels of CTFβ and its derivative A.

Although it cannot be ruled out that some alternative uncharacterized pathway may contribute to the parallel decrease of CTFβ and Aβ in PTPσ-deficient brains, these data consistently support the notion that PTPσ regulates APP amyloidogenic processing, likely via facilitation of BACE1 activity on APP, the initial process of Aβ production.

The Specificity of β-Amyloidogenic Regulation by PTPσ.

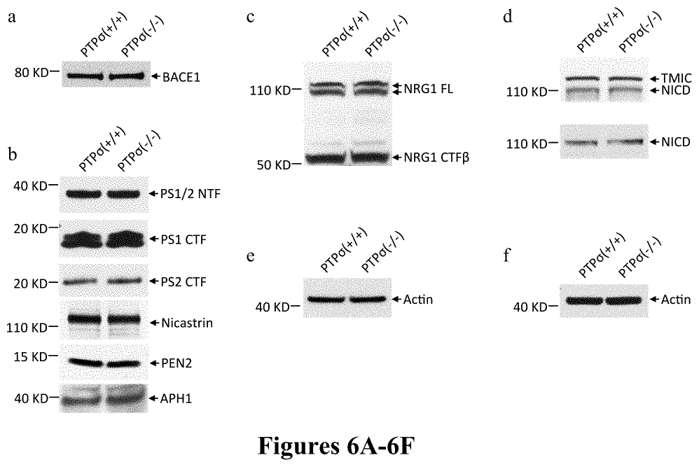

The constraining effect of PTPσ on APP amyloidogenic products led to further questions regarding whether this observation reflects a specific regulation of APP metabolism, or alternatively, a general modulation on the β- and γ-secretases. First, the expression level of these secretases in mouse brains were assessed with or without PTPσ. No change was found for BACE1 or the essential subunits of γ-secretase (FIG. 6a, b). Additionally, the question of whether PTPσ broadly modulates β- and γ-secretase activities was tested by examining the proteolytic processing of their other substrates. Besides APP, Neuregulin1 (NRG1)¹⁹⁻²¹ and Notch²²⁻²⁴ are the major in vivo substrates of BACE1 and γ-secretase, respectively. Neither BACE1 cleavage of NRG1 nor γ-secretase cleavage of Notch is affected by PTPσ deficiency (FIG. 6c, d). Taken together, these data rule out a generic modulation of β- and γ-secretases, but rather suggest a specificity of APP amyloidogenic regulation by PTPσ.

PTPσ Depletion Relieves Neuroinflammation and Synaptic Impairment in APP Transgenic Mice.

Substantial evidence from earlier studies has established that overproduction of Aβ in the brain elicits multiplex downstream pathological events, including chronic inflammatory responses of the glia, such as persistent astrogliosis. The reactive (inflammatory) glia would then crosstalk with neurons, evoking a vicious feedback loop that amplifies neurodegeneration during disease progression²⁵⁻²⁷.

The TgAPP-SwDI model is one of the earliest to develop neurodegenerative pathologies and behavioral deficits among many existing AD mouse models¹⁷. These mice were therefore chosen to further examine the role of PTPσ in AD pathologies downstream of neurotoxic A.

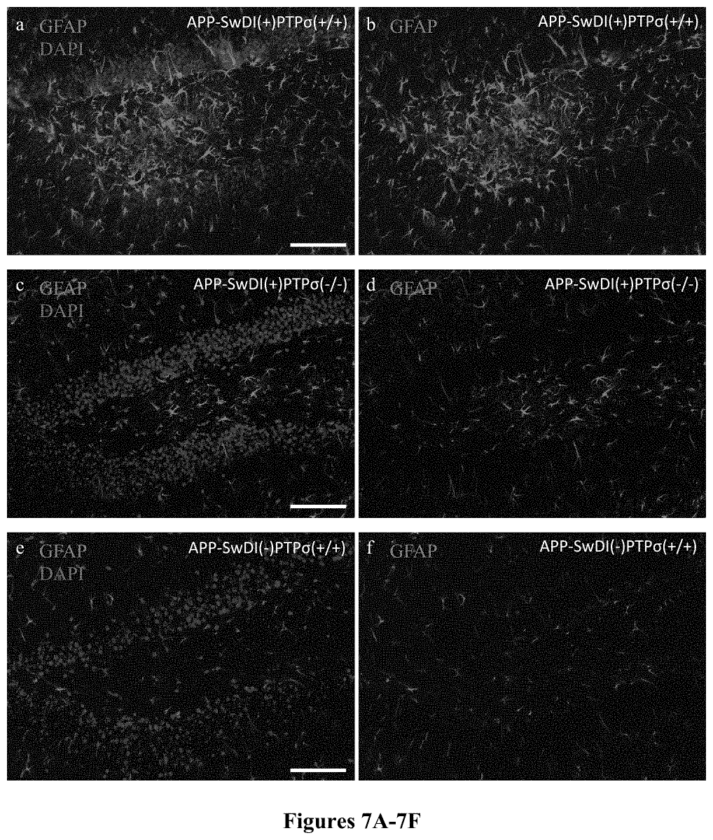

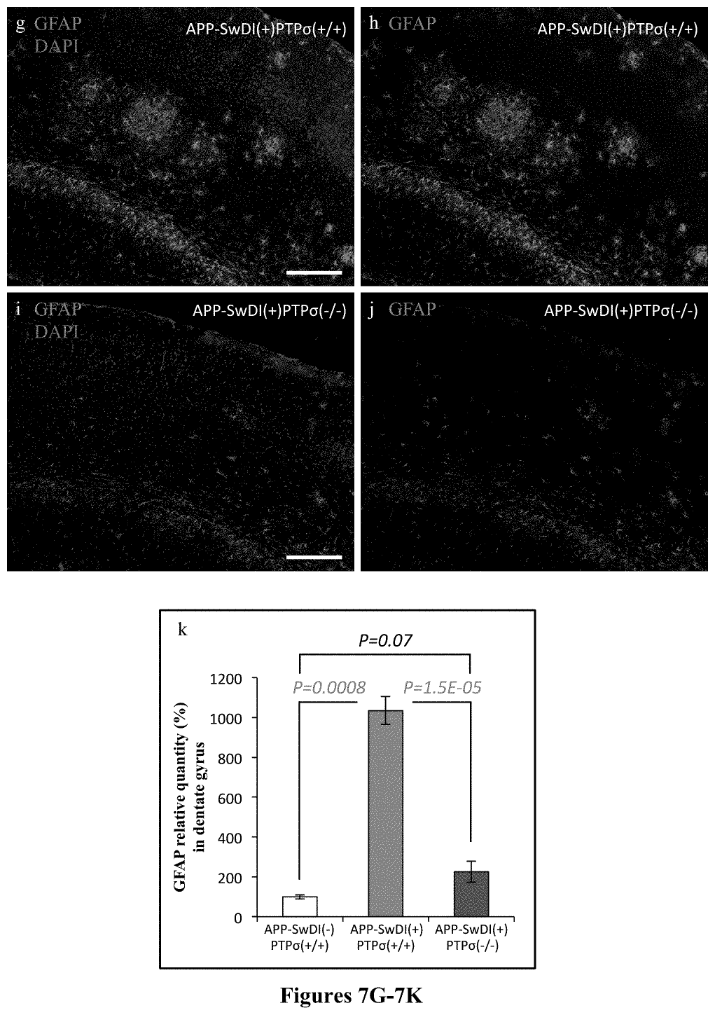

The APP-SwDI(+)PTPσ(+/+) mice, which express the TgAPP-SwDI transgene and wild type PTPσ, have developed severe neuroinflammation in the brain by the age of 9 months, as measured by the level of GFAP (glial fibrillary acidic protein), a marker of astrogliosis (FIG. 7). In the DG hilus, for example, GFAP expression level in the APP-SwDI(+)PTPσ(+/+) mice is more than tenfold compared to that in age-matched non-transgenic littermates [APP-SwDI(−) PTPσ(+/+)]. PTPσ deficiency, however, effectively attenuates astrogliosis induced by the amyloidogenic transgene. In the APP-SwDI(+)PTPσ(−/−) brains, depletion of PTPσ restores GFAβ expression in DG hilus back to a level close to that of non-transgenic wild type littermates (FIG. 7k).

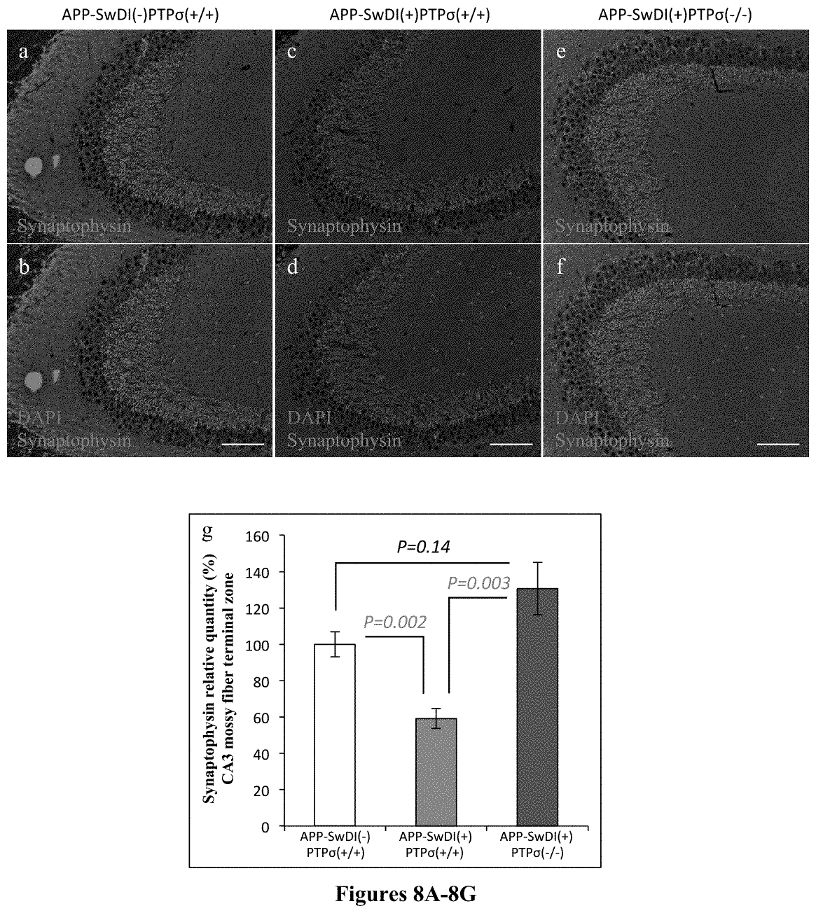

Among all brain regions, the most affected by the expression of TgAPP-SwDI transgene appears to be the hilus of the DG, where Aβ deposition and astrogliosis are both found to be the most severe (FIGS. 3g, h; FIG. 7). The question was therefore raised whether the pathologies in this area have an impact on the mossy fiber axons of DG pyramidal neurons, which project through the hilus into the CA3 region, where they synapse with the CA3 dendrites. Upon examining the presynaptic markers in CA3 mossy fiber terminal zone, decreased levels of Synaptophysin and Synapsin-1 were found in the APP-SwDI(+)PTPσ(+/+) mice, comparing to their age-matched non-transgenic littermates (FIG. 8, data not shown for Synapsin-1). Such synaptic impairment, evidently resulting from the expression of the APP transgene and possibly the overproduction of Aβ, is reversed by genetic depletion of PTPσ in the APP-SwDI(+)PTPσ(−/−) mice (FIG. 8).

Interestingly, the APP-SwDI(+)PTPσ(−/−) mice sometimes express higher levels of presynaptic markers in the CA3 terminal zone than their age-matched non-transgenic wild type littermates (FIG. 8g). This observation, although not statistically significant, may suggest an additional synaptic effect of PTPσ that is independent of the APP transgene, as observed in previous studies²⁸.

Tau Pathology in Aging AD Mouse Brains is Dependent on PTPσ.

Neurofibrillary tangles composed of hyperphosphorylated and aggregated Tau are commonly found in AD brains. These tangles tend to develop in a hierarchical pattern, appearing first in the entorhinal cortex before spreading to other brain regions^{5,6}. The precise mechanism of tangle formation, however, is poorly understood. The fact that Tau tangles and Aβ deposits can be found in separate locations in postmortem brains has led to the question of whether Tau pathology in AD is independent of Aβ accumulation^{5,6}. Additionally, despite severe cerebral β-amyloidosis in many APP transgenic mouse models, Tau tangles have not been reported, further questioning the relationship between Aβ and Tau pathologies in vivo.

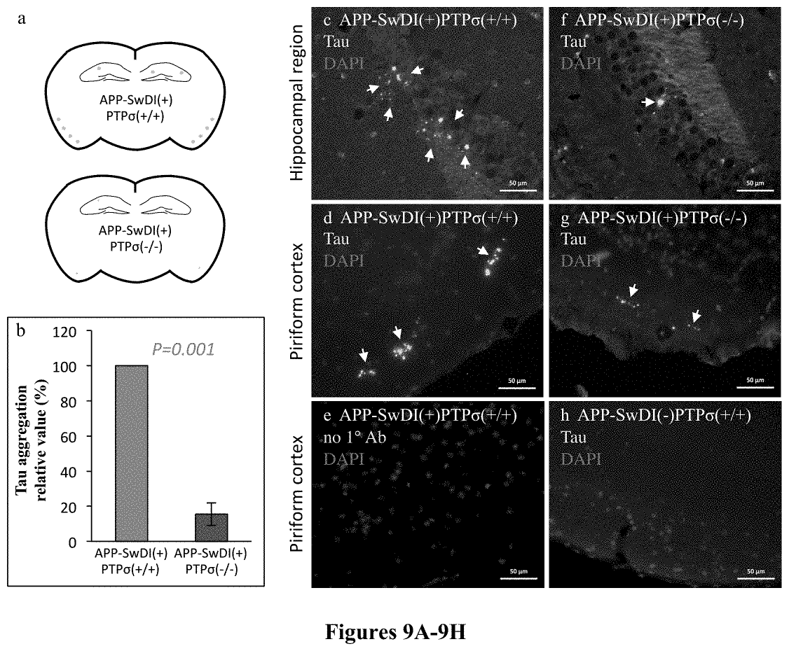

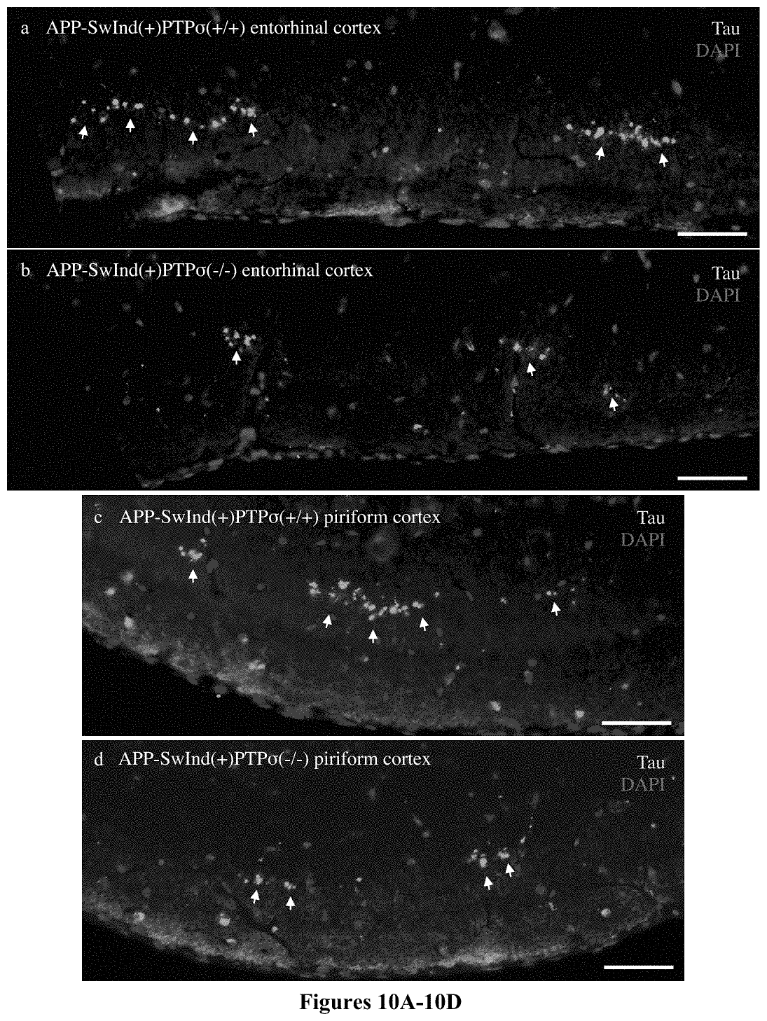

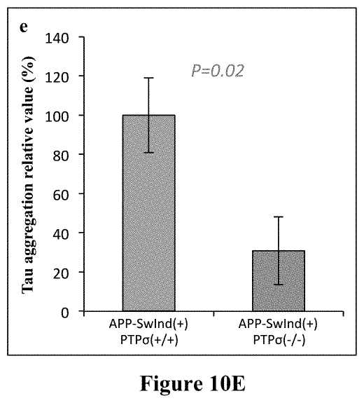

Nonetheless, a few studies did show non-tangle like assemblies of Tau in dystrophic neurites surrounding Aβ plaques in APP transgenic mouse lines²⁹⁻³¹, arguing that Aβ can be a causal factor for Tau dysregulation, despite that the precise nature of Tau pathologies may be different between human and mouse. In the histological analysis using an antibody against the proline-rich domain of Tau, Tau aggregation was observed in the brains of both TgAPP-SwDI and TgAPP-SwInd mice during the course of aging (around 9 months for the APP-SwDI(+)PTPσ(+/+) mice and 15 months for the APP-SwInd(+)PTPσ(+/+) mice) (FIG. 9; FIG. 10). Such aggregation is not seen in aged-matched non-transgenic littermates (FIG. 9h), suggesting that it is a pathological event downstream from the expression of amyloidogenic APP transgenes, possibly a result of Aβ cytotoxicity. Genetic depletion of PTPσ, which diminishes Aβ levels, suppresses Tau aggregation in both TgAPP-SwDI and TgAPP-SwInd mice (FIG. 9; FIG. 10).

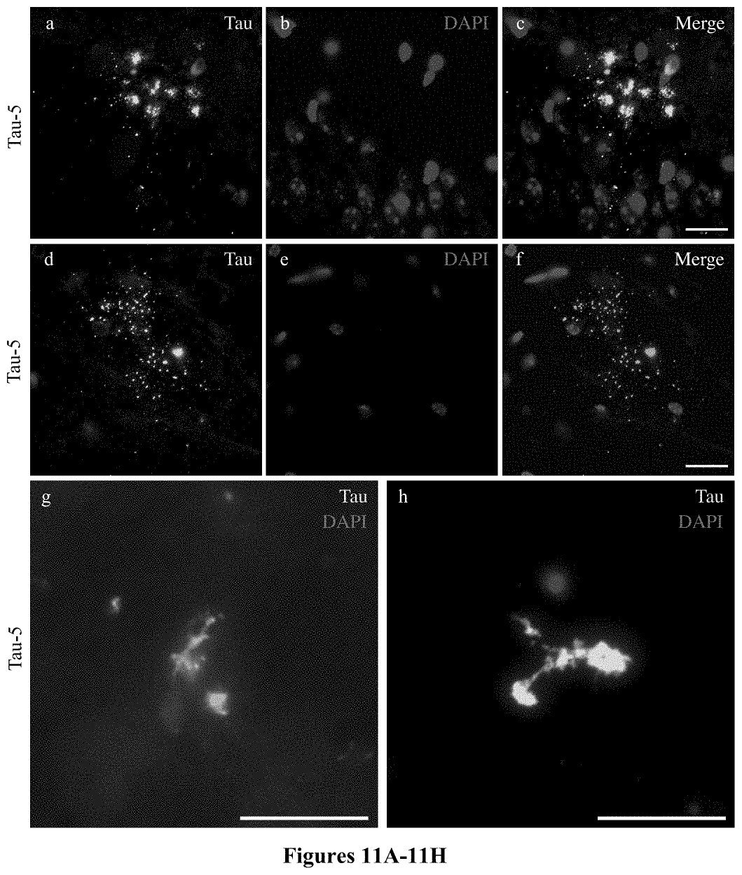

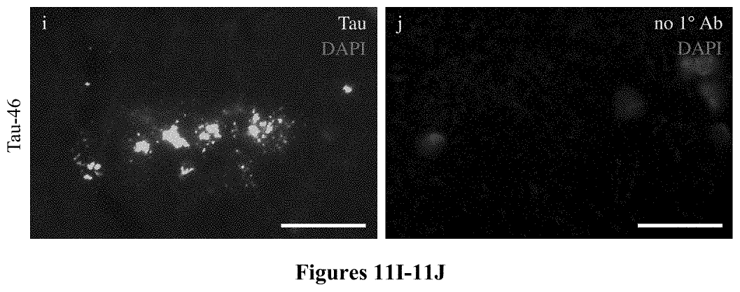

In both TgAPP-SwDI and TgAPP-SwInd mice, the Tau aggregates are found predominantly in the molecular layer of the piriform and entorhinal cortices, and occasionally in the hippocampal region (FIG. 9; FIG. 10), reminiscent of the early stage tangle locations in AD brains³². Upon closer examination, the Tau aggregates are often found in punctate shapes, likely in debris from degenerated cell bodies and neurites, scattered in areas free of nuclear staining (FIGS. 11a-f). Rarely, a few are in fibrillary structures, probably in degenerated cells before disassembling (FIG. 11g, h). To confirm these findings, an additional antibody was used to recognize the C-terminus of Tau. The same morphologies (FIG. 11i) and distribution pattern (FIG. 9a) were detected.

Consistent with the findings in postmortem AD brains, the distribution pattern of Tau aggregates in the TgAPP-SwDI brain does not correlate with that of Aβ deposition, which is pronounced in the hippocampus yet only sporadic in the piriform or entorhinal cortex at the age of 9 months (FIGS. 3g, h). Given that the causation of Tau pathology in these mice is possibly related to the overproduced Aβ, the segregation of predominant areas for Aβ and Tau depositions may indicate that the cytotoxicity originates from soluble Aβ instead of the deposited amyloid. It is also evident that neurons in different brain regions are not equally vulnerable to developing Tau pathology.

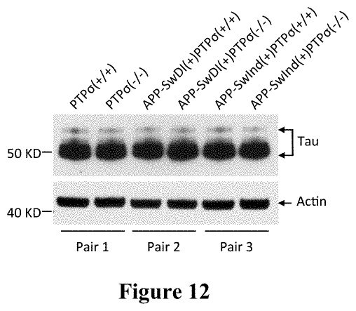

Next, the question of whether the expression of APP transgenes or genetic depletion of PTPσ regulates Tau aggregation by changing its expression level and/or phosphorylation status was examined. Western blot analysis of brain homogenates showed that Tau protein expression is not affected by the APP transgenes or PTPσ (FIG. 12), suggesting that the aggregation may result from local misfolding of Tau rather than an overexpression of this protein. These experiments with brain homogenates also revealed that TgAPP-SwDI or TgAPP-SwInd transgene, which apparently causes Tau aggregation, does not enhance the phosphorylation of Tau residues including Serine191, Therionine194, and Therionine220 (data not shown), whose homologues in human Tau (Serine202, Therionine205, and Therionine231) are typically hyperphosphorylated in neurofibrillary tangles. These findings are consistent with a recent quantitative study showing similar post-translational modifications of Tau in wild type and TgAPP-SwInd mice³³. Furthermore, unlike previously reported^{29,30}, we could not detect these phosphorylated residues in the Tau aggregates, suggesting that the epitopes are either missing (residues not phosphorylated or cleaved off) or embedded inside the misfolding. Given the complexity of Tau post-translational modification, one cannot rule out that the aggregation may be mediated by some unidentified modification(s) of Tau. It is also possible that other factors, such as molecules that bind to Tau, may precipitate the aggregation.

Although the underlying mechanism is still unclear, the finding of Tau pathology in these mice establishes a causal link between the expression of amyloidogenic APP transgenes and a dysregulation of Tau assembly. The data also suggest a possibility that PTPσ depletion may suppress Tau aggregation by reducing amyloidogenic products of APP.

Malfunction of Tau is broadly recognized as a neurodegenerative marker since it indicates microtubule deterioration⁷. The constraining effect on Tau aggregation by genetic depletion of PTPσ thus provides additional evidence for the role of this receptor as a pivotal regulator of neuronal integrity.

PTPσ Deficiency Rescues Behavioral Deficits in AD Mouse Models.

Next, the question was assessed of whether the alleviation of neuropathologies by PTPσ depletion is accompanied with a rescue from AD relevant behavioral deficits. The most common symptoms of AD include short-term memory loss and apathy among the earliest, followed by spatial disorientation amid impairment of many cognitive functions as the dementia progresses. Using Y maze and novel object assays as surrogate models, these cognitive and psychiatric features were evaluated in the TgAPP-SwDI and TgAPP-SwInd mice.

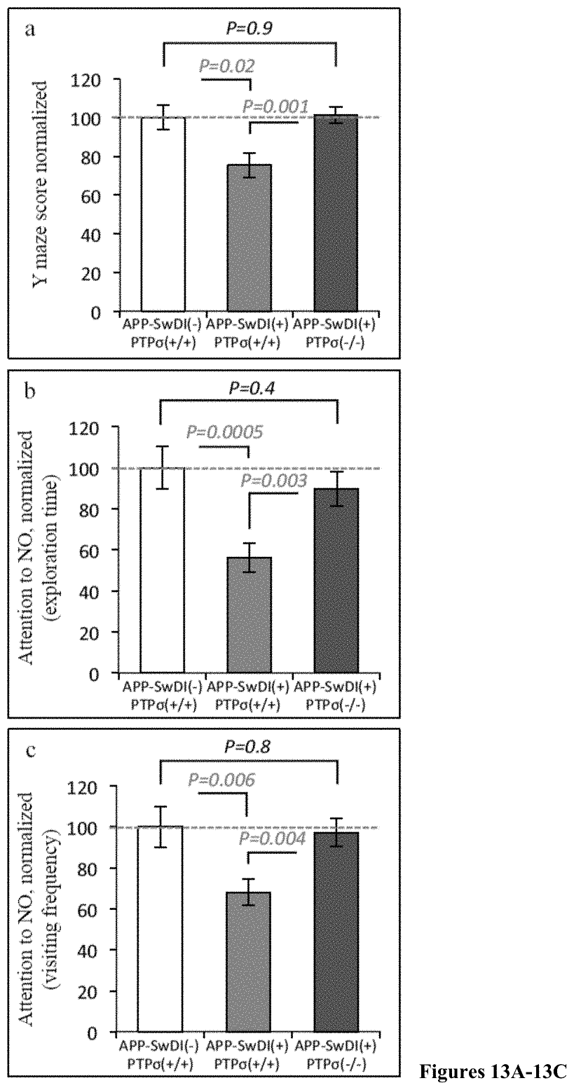

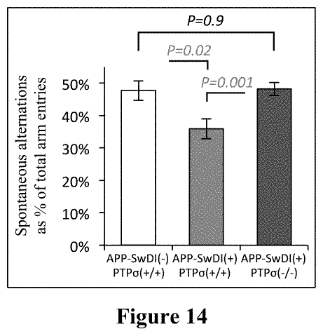

The Y-maze assay, which allows mice to freely explore three identical arms, measures their short-term spatial memory. It is based on the natural tendency of mice to alternate arm exploration without repetitions. The performance is scored by the percentage of spontaneous alternations among total arm entries, and a higher score indicates better spatial navigation. Compared to the non-transgenic wild type mice within the colony, the APP-SwDI(+)PTPσ(+/+) mice show a clear deficit in their performance. Genetic depletion of PTPσ in the APP-SwDI(+)PTPσ(−/−) mice, however, unequivocally restores the cognitive performance back to the level of non-transgenic wild type mice (FIG. 13a, FIG. 14).

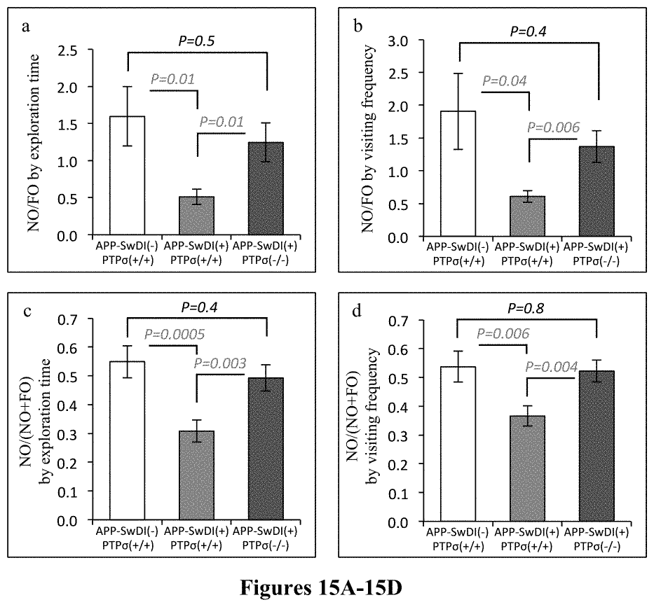

Apathy, the most common neuropsychiatric symptom reported among individuals with AD, is characterized by a loss of motivation and diminished attention to novelty, and has been increasingly adopted into early diagnosis of preclinical and early prodromal AD³⁴⁻³⁶. Many patients in early stage AD lose attention to novel aspects of their environment despite their ability to identify novel stimuli, suggesting an underlying defect in the circuitry responsible for further processing of the novel information^{34,35}. As a key feature of apathy, such deficits in attention to novelty can be accessed by the “curiosity figures task” or the “oddball task” in patients^{34,35,37}. These visual-based novelty encoding tasks are very similar to the novel object assay for rodents, which measures the interest of animals in a novel object (NO) when they are exposed simultaneously to a prefamiliarized object (FO). This assay was therefore used to test the attention to novelty in the APP transgenic mice. When mice are pre-trained to recognize the FO, their attention to novelty is then measured by the discrimination index denoted as the ratio of NO exploration to total object exploration (NO+FO), or alternatively, by the ratio of NO exploration to FO exploration. Whereas both ratios are commonly used, a combination of these assessments provides a more comprehensive evaluation of animal behavior. In this test, as indicated by both measurements, the expression of APP-SwDI transgene in the APP-SwDI(+)PTPσ(+/+) mice leads to a substantial decrease in NO exploration as compared to non-transgenic wild type mice (FIG. 11b, c; FIG. 15). Judging by their NO/FO ratios, it is evident that both the transgenic and non-transgenic groups are able to recognize and differentiate between the two objects (FIG. 15a, b). Thus, the reduced NO exploration by the APP-SwDI(+)PTPσ(+/+) mice may reflect a lack of interest in the NO or an inability to shift attention to the NO. Once again, this behavioral deficit is largely reversed by PTPσ deficiency in the APP-SwDI(+)PTPσ(−/−) mice (FIG. 13b, c; FIG. 15), consistent with previous observation of increased NO preference in the absence of PTPσ²⁸.

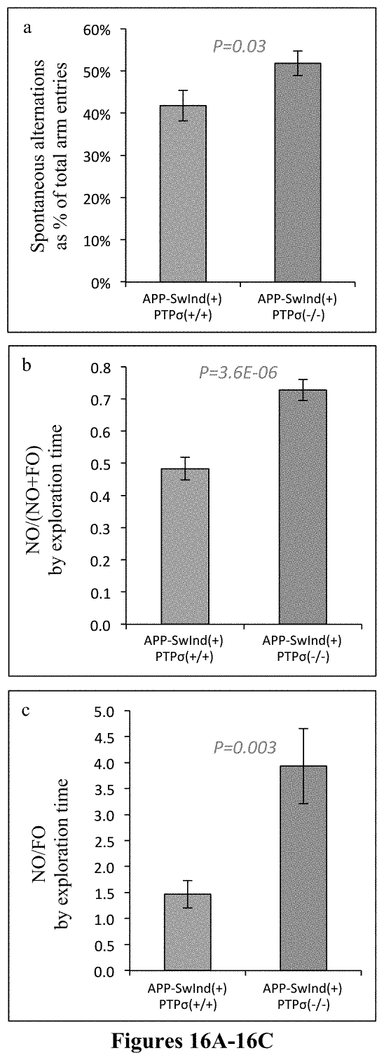

To further verify the effects of PTPσ on these behavioral aspects, the TgAPP-SwInd mice were also tested using both assays, and similar results were observed. This confirms an improvement on both short-term spatial memory and attention to novelty upon genetic depletion of PTPσ (FIG. 16).

Discussion

The above data showed that β-amyloidosis and several downstream disease features are dependent on PTPσ in two mouse models of genetically inherited AD. This form of AD develops inevitably in people who carry gene mutations that promote amyloidogenic processing of APP and overproduction of A. The data presented herein suggest that targeting PTPσ is a potential therapeutic approach that could overcome such dominant genetic driving forces to curtail AD progression. The advantage of this targeting strategy is that it suppresses Aβ accumulation without broadly affecting other major substrates of the β- and γ-secretases, thus predicting a more promising translational potential as compared to those in clinical trials that generically inhibit the secretases.

PTPσ was previously characterized as a neuronal receptor of the chondroitin sulfate- and heparan sulfate-proteoglycans (CSPGs and HSPGs)^{10,11}. In response to these two classes of extracellular ligands, PTPσ functions as a “molecular switch” by regulating neuronal behavior in opposite manners⁸. The finding presented herein of a pivotal role for the proteoglycan sensor PTPσ in AD pathogenesis may therefore implicate an involvement of the perineuronal matrix in AD etiology.

More than 95% of AD cases are sporadic, which are not genetically inherited but likely result from insults to the brain that occurred earlier in life. AD risk factors, such as traumatic brain injury and cerebral ischemia³⁸⁻⁴¹ have been shown to induce overproduction of Aβ in both human and rodents⁴²⁻⁴⁶ and speed up progression of this dementia in animal models⁴⁷⁻⁴⁹. However, what promotes the amyloidogenic processing of APP in these cases is still a missing piece of the puzzle in understanding the AD-causing effects of these notorious risk factors.

Coincidently, both traumatic brain injury and cerebral ischemia cause pronounced remodeling of the perineuronal microenvironment at lesion sites, marked by increased expression of CSPGs⁵⁰⁻⁵³, a major component of the perineuronal net that is upregulated during neuroinflammation and glial scar formation⁵⁴⁻⁵⁶. In the brains of AD patients, CSPGs were found associated with Aβ depositions, further suggesting an uncanny involvement of these proteoglycans in AD development⁵⁷. On the other hand, analogues of heparan sulfate (HS, carbohydrate side chains of HSPGs that bind to PTPσ) were shown to inhibit BACE1 activity, suggesting their function in preventing Aβ overproduction⁵⁸. After cerebral ischemia, however, the expression of Heparanase, an enzyme that degrades HS, was found markedly increased⁵⁹. Collectively, these findings suggest a disrupted molecular balance between CSPGs and HSPGs in brains after lesion, which may ignite insidious signaling cascades preceding the onset of AD.

Further study could include investigation of a potential mechanism, whereby chronic CSPG upregulation or HSPG degradation in lesioned brains may sustain aberrant signaling through their neuronal sensor PTPσ, leading to biased processing of APP and a neurotoxic “Aβ cascade”. As such, altered signaling from PTPσ after traumatic brain injury and ischemic stroke may explain how these risk factors can trigger subsequent onset of AD. Restoring the integrity of brain microenvironment therefore could be essential in preventing AD for the population at risk.

### Example 2: CS and HS Regulates APP Amyloidogenic Processing in Opposite Manners

CS and HS/heparin are two classes of PTPσ ligands in the perineuronal space that compete for binding to the same site on receptor PTPσ with similar affinities⁸. Increased CS/HS ratio is often found after brain injuries or ischemic stroke^{50-53,59}, both of which are prominent risk factors for AD and alike neurodegenerative diseases.

These two classes of ligands were shown previously to oppositely regulate neuronal responses, such as neurite outgrowth, through their common receptor PTPσ. Whereas CS inhibits neurite outgrowth, HS/heparin promotes neurite outgrowth.

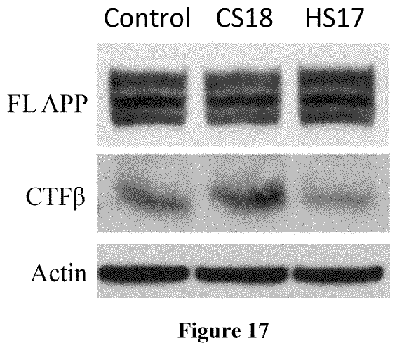

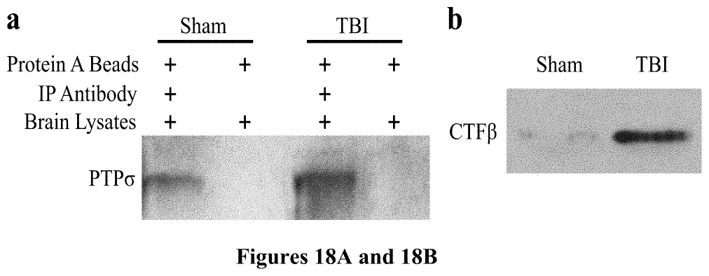

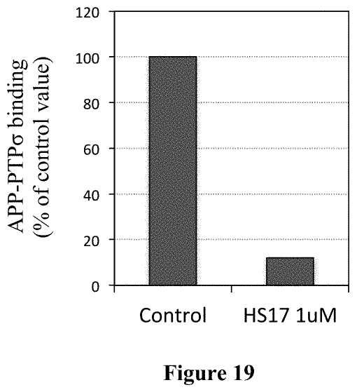

When tested in an in vitro assay for their effects on APP amyloidogenic processing, these PTPσ ligands again showed opposite effects. As in FIG. 17, incubation of cell membrane preparations extracted from fresh mouse brain homogenates with these PTPσ ligands results in an increased level of APP β-cleavage by CS, but a decreased level of APP β-cleavage by HS/heparin. Whereas CS levels are well documented to be upregulated after traumatic brain injury (TBI) in rats and mice, this study found increased APP-PTPσ binding accompanied with significantly enhanced level of APP β-cleavage product (CTFβ) in injured brains (FIG. 18). On the contrary, HS/heparin, which inhibits APP n-cleavage, effectively disrupts APP-PTPσ binding (FIG. 19). These data thus suggest that the molecular balance of PTPσ ligands CS and HS in the brain is important in regulating APP amyloidogenic processing, and that the promoting and suppressing effects on APP n-cleavage by CS and HS, respectively, are mediated by their control on APP-PTPσ binding.

### Example 3: Defining Binding Regions on Human APP and PTPσ

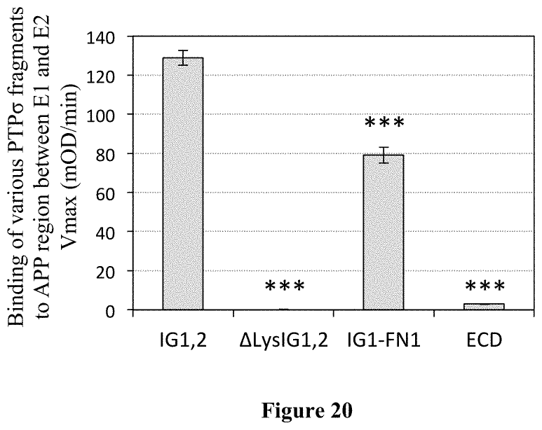

Domain regions were subcloned from human APP695 (construct by Denis Selkoe and Tracy Yang labs purchased through Addgene.com) and PTPσ (constructs from Radu Aricescu lab). Recombinant APP and PTPσ proteins were tested in solid phase ELISA binding assays to define the binding regions on each partner. Neither E1 or E2 domain of APP interacts with PTPσ (data not shown), however the region in between these two APP domains (SEQ ID NO:1) appears to have high affinity with PTPσ IG1 domain (FIG. 20). The lysine residues (K67, 68, 70, 71) in PTPσ IG1 ligand binding site, which was shown to be responsible for CS and HS binding^{8,11,60} are also important for its interaction with APP, as mutation of these residues abolishes APP-PTPσ binding. Comparing APP binding strength of difference PTPσ fragments, it appears that inclusion of the fibronectin (FN) domains of PTPσ weakens the interaction with APP, likely due to folding of PTPσ that covers up the ligand binding site in its IG1 domain⁶¹. Full PTPσ extracellular domain nearly lost binding with APP SEQ ID NO:1, suggesting that factors triggering the unfold PTPσ are required for APP-PTPσ binding.

Sequences:

Sequences for the peptides used in Example 3 are provided in Tables 3, 4, and 5.

TABLE 3
Peptides derived from APP

| | |
|---|---|
| SEQ ID NO: 101 | ADAEEDDSDVW |
| SEQ ID NO: 112 | WGGADTDYADG |
| SEQ ID NO: 388 | EDKVVEVAEEEEVA |
| SEQ ID NO: 139 | VEEEEADDDED |
| SEQ ID NO: 151 | EDGDEVEEEAE |
| SEQ ID NO: 157 | EEEAEEPYEEA |
| SEQ ID NO: 251 | EPYEEATERTTS |
| SEQ ID NO: 897 | ESVEEVVRVPTTA |
| SEQ ID NO: 900 | ATERTTSIATTTTTTTESVEEVVR |

TABLE 4
Peptides derived from PTPσ

| | |
|---|---|
| SEQ ID NO: 655 | TWNKKGKKVNSQ |
| SEQ ID NO: 769 | RIQPLRTPRDENV |
| SEQ ID NO: 898 | KKGKK |
| SEQ ID NO: 899 | RTPR |

TABLE 5
Membrane penetrating peptides

| | |
|---|---|
| SEQ ID NO: 895 | GRKKRRQRRRPQ |
| SEQ ID NO: 896 | RKKRRQRRRC |

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

### REFERENCES

1. Selkoe, D. J. Alzheimer's disease. Cold Spring Harbor perspectives in biology 3(2011).
2. Yan, R. & Vassar, R. Targeting the beta secretase BACE1 for Alzheimer's disease therapy.

The Lancet. Neurology 13, 319-329 (2014).
3. Mikulca, J. A., et al. Potential novel targets for Alzheimer pharmacotherapy: II. Update on secretase inhibitors and related approaches. Journal of clinical pharmacy and therapeutics 39, 25-37 (2014).
4. De Strooper, B. Lessons from a failed gamma-secretase Alzheimer trial. Cell 159, 721-726 (2014).
5. Arriagada, P. V., Growdon, J. H., Hedley-Whyte, E. T. & Hyman, B. T. Neurofibrillary tangles but not senile plaques parallel duration and severity of Alzheimer's disease. Neurology 42, 631-639 (1992).
6. Bouras, C., Hof, P. R., Giannakopoulos, P., Michel, J. P. & Morrison, J. H. Regional distribution of neurofibrillary tangles and senile plaques in the cerebral cortex of elderly patients: a quantitative evaluation of a one-year autopsy population from a geriatric hospital. Cerebral cortex 4, 138-150 (1994).
7. Wang, Y. & Mandelkow, E. Tau in physiology and pathology. Nature reviews. Neuroscience 17, 5-21 (2016).
8. Coles, C. H., et al. Proteoglycan-specific molecular switch for RPTPsigma clustering and neuronal extension. Science 332, 484-488 (2011).
9. Elchebly, M., et al. Neuroendocrine dysplasia in mice lacking protein tyrosine phosphatase sigma. Nature genetics 21, 330-333 (1999).
10. Aricescu, A. R., McKinnell, I. W., Halfter, W. & Stoker, A. W. Heparan sulfate proteoglycans are ligands for receptor protein tyrosine phosphatase sigma. Molecular and cellular biology 22, 1881-1892 (2002).
11. Shen, Y, et al. PTPsigma is a receptor for chondroitin sulfate proteoglycan, an inhibitor of neural regeneration. Science 326, 592-596 (2009).
12. Wang, H., et al. Expression of receptor protein tyrosine phosphatase-sigma (RPTP-sigma) in the nervous system of the developing and adult rat. Journal of neuroscience research 41, 297-310 (1995).
13. Yan, H., et al. A novel receptor tyrosine phosphatase-sigma that is highly expressed in the nervous system. The Journal of biological chemistry 268, 24880-24886 (1993).
14. Chow, V. W., Mattson, M. P., Wong, P. C. & Gleichmann, M. An overview of APP processing enzymes and products. Neuromolecular medicine 12, 1-12 (2010).
15. Nunan, J. & Small, D. H. Regulation of APP cleavage by alpha-, beta- and gamma-secretases. FEBS letters 483, 6-10 (2000).
16. Estus, S., et al. Potentially amyloidogenic, carboxyl-terminal derivatives of the amyloid protein precursor. Science 255, 726-728 (1992).
17. Davis, J., et al. Early-onset and robust cerebral microvascular accumulation of amyloid beta-protein in transgenic mice expressing low levels of a vasculotropic Dutch/Iowa mutant form of amyloid beta-protein precursor. The Journal of biological chemistry 279, 20296-20306 (2004).
18. Mucke, L., et al. High-level neuronal expression of abeta 1-42 in wild-type human amyloid protein precursor transgenic mice: synaptotoxicity without plaque formation. The Journal of neuroscience: the official journal of the Society for Neuroscience 20, 4050-4058 (2000).
19. Fleck, D., Garratt, A. N., Haass, C. & Willem, M. BACE1 dependent neuregulin processing: review. Current Alzheimer research 9, 178-183 (2012).
20. Luo, X., et al. Cleavage of neuregulin-1 by BACE1 or ADAM10 protein produces differential effects on myelination. The Journal of biological chemistry 286, 23967-23974 (2011).
21. Cheret, C., et al. Bacel and Neuregulin-1 cooperate to control formation and maintenance of muscle spindles. The EMBO journal 32, 2015-2028 (2013).
22. De Strooper, B., et al. A presenilin-1-dependent gamma-secretase-like protease mediates release of Notch intracellular domain. Nature 398, 518-522 (1999).
23. Tian, Y, Bassit, B., Chau, D. & Li, Y. M. An APP inhibitory domain containing the Flemish mutation residue modulates gamma-secretase activity for Abeta production. Nature structural & molecular biology 17, 151-158 (2010).
24. Zhang, Z., et al. Presenilins are required for gamma-secretase cleavage of beta-APP and transmembrane cleavage of Notch-1. Nature cell biology 2, 463-465 (2000).
25. Glass, C. K., Saijo, K., Winner, B., Marchetto, M. C. & Gage, F. H. Mechanisms underlying inflammation in neurodegeneration. Cell 140, 918-934 (2010).
26. DeWitt, D. A., Perry, G., Cohen, M., Doller, C. & Silver, J. Astrocytes regulate microglial phagocytosis of senile plaque cores of Alzheimer's disease. Experimental neurology 149, 329-340 (1998).
27. Frederickson, R. C. Astroglia in Alzheimer's disease. Neurobiology of aging 13, 239-253 (1992).
28. Horn, K. E., et al. Receptor protein tyrosine phosphatase sigma regulates synapse structure, function and plasticity. Journal of neurochemistry 122, 147-161 (2012).
29. Tomidokoro, Y, et al. Abeta amyloidosis induces the initial stage of tau accumulation in APP(Sw) mice. Neuroscience letters 299, 169-172 (2001).
30. Sturchler-Pierrat, C., et al. Two amyloid precursor protein transgenic mouse models with Alzheimer disease-like pathology. Proceedings of the National Academy of Sciences of the United States of America 94, 13287-13292 (1997).
31. Rockenstein, E., Mallory, M., Mante, M., Sisk, A. & Masliaha, E. Early formation of mature amyloid-beta protein deposits in a mutant APP transgenic model depends on levels of Abeta(1-42). Journal of neuroscience research 66, 573-582 (2001).
32. Holtzman, D. M., et al. Tau: From research to clinical development. Alzheimer's & dementia: the journal of the Alzheimer's Association (2016).
33. Morris, M., et al. Tau post-translational modifications in wild-type and human amyloid precursor protein transgenic mice. Nature neuroscience 18, 1183-1189 (2015).
34. Daffner, K. R., et al. Pathophysiology underlying diminished attention to novel events in patients with early AD. Neurology 56, 1377-1383 (2001).
35. Daffner, K. R., Mesulam, M. M., Cohen, L. G. & Scinto, L. F. Mechanisms underlying diminished novelty-seeking behavior in patients with probable Alzheimer's disease. Neuropsychiatry, neuropsychology, and behavioral neurology 12, 58-66 (1999).
36. Marin, R. S., Biedrzycki, R. C. & Firinciogullari, S. Reliability and validity of the Apathy Evaluation Scale. Psychiatry research 38, 143-162 (1991).
37. Kaufman, D. A., Bowers, D., Okun, M. S., Van Patten, R. & Perlstein, W. M. Apathy, Novelty Processing, and the P3 Potential in Parkinson's Disease. Frontiers in neurology 7, 95 (2016).
38. Johnson, V. E., Stewart, W. & Smith, D. H. Traumatic brain injury and amyloid-beta pathology: a link to Alzheimer's disease? Nature reviews. Neuroscience 11, 361-370 (2010).
39. Sivanandam, T. M. & Thakur, M. K. Traumatic brain injury: a risk factor for Alzheimer's disease. Neuroscience and biobehavioral reviews 36, 1376-1381 (2012).
40. Kalaria, R. N. The role of cerebral ischemia in Alzheimer's disease. Neurobiology of aging 21, 321-330 (2000).
41. Cole, S. L. & Vassar, R. Linking vascular disorders and Alzheimer's disease: potential involvement of BACE1. Neurobiology of aging 30, 1535-1544 (2009).
42. Emmerling, M. R., et al. Traumatic brain injury elevates the Alzheimer's amyloid peptide A beta 42 in human CSF. A possible role for nerve cell injury. Annals of the New York Academy of Sciences 903, 118-122 (2000).
43. Olsson, A., et al. Marked increase of beta-amyloid(1-42) and amyloid precursor protein in ventricular cerebrospinal fluid after severe traumatic brain injury. Journal of neurology 251, 870-876 (2004).
44. Loane, D. J., et al. Amyloid precursor protein secretases as therapeutic targets for traumatic brain injury. Nature medicine 15, 377-379 (2009).
45. Pluta, R., Furmaga-Jablonska, W., Maciejewski, R., Ulamek-Koziol, M. & Jablonski, M. Brain ischemia activates beta- and gamma-secretase cleavage of amyloid precursor protein: significance in sporadic Alzheimer's disease. Molecular neurobiology 47, 425-434 (2013).
46. Washington, P. M., et al. The effect of injury severity on behavior: a phenotypic study of cognitive and emotional deficits after mild, moderate, and severe controlled cortical impact injury in mice. Journal of neurotrauma 29, 2283-2296 (2012).
47. Kokiko-Cochran, O., et al. Altered Neuroinflammation and Behavior after Traumatic Brain Injury in a Mouse Model of Alzheimer's Disease. Journal of neurotrauma (2015).
48. Tajiri, N., Kellogg, S. L., Shimizu, T., Arendash, G. W. & Borlongan, C. V. Traumatic brain injury precipitates cognitive impairment and extracellular Abeta aggregation in Alzheimer's disease transgenic mice. PloS one 8, e78851 (2013).
49. Watanabe, T., Takasaki, K., Yamagata, N., Fujiwara, M. & Iwasaki, K. Facilitation of memory impairment and cholinergic disturbance in a mouse model of Alzheimer's disease by mild ischemic burden. Neuroscience letters 536, 74-79 (2013).
50. Properzi, F., et al. Chondroitin 6-sulphate synthesis is up-regulated in injured CNS, induced by injury-related cytokines and enhanced in axon-growth inhibitory glia. The European journal of neuroscience 21, 378-390 (2005).
51. Yi, J. H., et al. Alterations in sulfated chondroitin glycosaminoglycans following controlled cortical impact injury in mice. The Journal of comparative neurology 520, 3295-3313 (2012).
52. Hill, J. J., Jin, K., Mao, X. O., Xie, L. & Greenberg, D. A. Intracerebral chondroitinase ABC and heparan sulfate proteoglycan glypican improve outcome from chronic stroke in rats. Proceedings of the National Academy of Sciences of the United States of America 109, 9155-9160 (2012).
53. Huang, L., et al. Glial scar formation occurs in the human brain after ischemic stroke. International journal of medical sciences 11, 344-348 (2014).
54. Celio, M. R. & Blumcke, I. Perineuronal nets—a specialized form of extracellular matrix in the adult nervous system. Brain research. Brain research reviews 19, 128-145 (1994).
55. Cregg, J. M., et al. Functional regeneration beyond the glial scar. Experimental neurology 253, 197-207 (2014).
56. Soleman, S., Filippov, M. A., Dityatev, A. & Fawcett, J. W. Targeting the neural extracellular matrix in neurological disorders. Neuroscience 253, 194-213 (2013).
57. DeWitt, D. A., Silver, J., Canning, D. R. & Perry, G. Chondroitin sulfate proteoglycans are associated with the lesions of Alzheimer's disease. Experimental neurology 121, 149-152 (1993).
58. Patey, S. J., Edwards, E. A., Yates, E. A. & Turnbull, J. E. Heparin derivatives as inhibitors of BACE-1, the Alzheimer's beta-secretase, with reduced activity against factor Xa and other proteases. Journal of medicinal chemistry 49, 6129-6132 (2006).
59. Li, J., et al. Expression of heparanase in vascular cells and astrocytes of the mouse brain after focal cerebral ischemia. Brain research 1433, 137-144 (2012).
60. Sajnani-Perez, G., Chilton, J. K., Aricescu, A. R., Haj, F. & Stoker, A. W. Isoform-specific binding of the tyrosine phosphatase PTPsigma to a ligand in developing muscle. Molecular and cellular neurosciences 22, 37-48 (2003).
61. Coles, C. H., et al. Structural basis for extracellular cis and trans RPTPsigma signal competition in synaptogenesis. Nature communications 5, 5209 (2014).

1. A non-naturally occurring fusion peptide for treating or preventing a neurodegenerative disorder, the peptide comprising;
a decoy fragment of Receptor Protein Tyrosine Phosphatase Sigma (PTPσ), and
a blood brain barrier penetrating sequence;
wherein the decoy fragment of PTPσ comprises the amino acid positions 34-82 of sequence SEQ ID NO: 442, the amino acid positions 34-48 of sequence SEQ ID NO: 442, the amino acid positions 34-54 of sequence SEQ ID NO: 442, the amino acid positions 34-58 of sequence SEQ ID NO: 442, the amino acid positions 34-64 of sequence SEQ ID NO: 442, the amino acid positions 34-73 of sequence SEQ ID NO: 442, the amino acid positions 39-54 of sequence SEQ ID NO: 442, the amino acid positions 39-58 of sequence SEQ ID NO: 442, the amino acid positions 39-64 of sequence SEQ ID NO: 442, the amino acid positions 39-73 of sequence SEQ ID NO: 442, the amino acid positions 39-82 of sequence SEQ ID NO: 442, the amino acid sequence SEQ ID NO: 491, the amino acid positions 49-64 of sequence SEQ ID NO: 442, the amino acid positions 49-73 of sequence SEQ ID NO: 442, the amino acid positions 49-82 of sequence SEQ ID NO: 442, the amino acid sequence SEQ ID NO: 497, the amino acid positions 55-73 of sequence SEQ ID NO: 442, the amino acid positions 55-82 of sequence SEQ ID NO: 442, the amino acid positions 59-73 of sequence SEQ ID NO: 442, or the amino acid positions 59-82 of sequence SEQ ID NO: 442.

2. The peptide of claim 1, wherein the decoy fragment of PTPσ is a peptide comprising the amino acid positions 34-82 of sequence SEQ ID NO: 442, the amino acid positions 34-48 of sequence SEQ ID NO: 442, the amino acid positions 34-54 of sequence SEQ ID NO: 442, the amino acid positions 34-58 of sequence SEQ ID NO: 442, the amino acid positions 34-64 of sequence SEQ ID NO: 442, or the amino acid positions 34-73 of sequence SEQ ID NO: 442.

3. The peptide of claim 1, wherein the decoy fragment of PTPσ is a peptide comprising the amino acid positions 39-54 of sequence SEQ ID NO: 442, the amino acid positions 39-58 of sequence SEQ ID NO: 442, the amino acid positions 39-64 of sequence SEQ ID NO: 442, the amino acid positions 39-73 of sequence SEQ ID NO: 442, or the amino acid positions 39-82 of sequence SEQ ID NO: 442.

4. (canceled)

5. The peptide of claim 1, wherein the decoy fragment of PTPσ is a peptide comprising the amino acid sequence SEQ ID NO: 491, the amino acid positions 49-64 of sequence SEQ ID NO: 442, the amino acid positions 49-73 of sequence SEQ ID NO: 442, or the amino acid positions 49-82 of sequence SEQ ID NO: 442.

6. The peptide of claim 1, wherein the decoy fragment of PTPσ is a peptide comprising the amino acid sequence SEQ ID NO: 497, the amino acid positions 55-73 of sequence SEQ ID NO: 442, or the amino acid positions 55-82 of sequence SEQ ID NO: 442.

7. The peptide of claim 1, wherein the decoy fragment of PTPσ comprises is a peptide comprising the amino acid positions 59-73 of sequence SEQ ID NO: 442, or the amino acid positions 59-82 of sequence SEQ ID NO: 442.

8. The peptide of claim 1, wherein the blood brain barrier penetrating sequence comprises amino acid sequence SEQ ID NO: 880, SEQ ID NO: 883, SEQ ID NO: 888, SEQ ID NO: 894, SEQ ID NO: 895, SEQ ID NO: 896.

9. The peptide of claim 1, wherein the peptide is cyclic.

10. A composition, comprising the peptide of claim 1 and further comprising a pharmaceutically acceptable excipient.

11.-21. (canceled)

22. A method of treating a neurodegenerative disorder in a subject, the method comprising administering to the subject a composition of claim 10.

23. The method of claim 22, wherein the neurodegenerative disease is selected from the group consisting of Alzheimer's Disease, Lewy body dementia, frontotemporal dementia, cerebral amyloid angiopathy, primary age-related tauopathy, chronic traumatic encephalopathy, Parkinson's disease, postencephalitic parkinsonism, Huntington's disease, amyolateral sclerosis, Pick's disease, progressive supranuclear palsy, corticobasal degeneration, Lytico-Bodig disease, ganglioglioma and gangliocytoma, subacute sclerosing panencephalitis, Hallervorden-Spatz disease, and/or Creutzfeldt-Jakob disease.

24. (canceled)

25. A method of preventing a neurodegenerative disorder in an at-risk subject, the method comprising administering to the subject a composition that interferes with the binding of Amyloid Precursor Protein (APP) to Receptor Protein Tyrosine Phosphatase Sigma (PTPσ), wherein the at-risk subject is at age older than 60 years or has received a medical diagnosis associated with Down syndrome, brain injury, or cerebral ischemia.

26. The method of claim 25, wherein the composition comprises the composition of claim 10.

27.-34. (canceled)
